(19) United States
(12) Patent Application Publication
(10) Pub. No.: US 2025/0302291 A1
(43) Pub. Date: Oct. 2, 2025

(54) SURGICAL INSTRUMENT

(71) Applicant: LIVSMED INC., Seongnam-si (KR)

(72) Inventors: Dong Hoon KANG, Seoul (KR); Tae Jin PARK, Seongnam-si (KR); Hyun JOO, Seoul (KR)

(21) Appl. No.: 19/092,558

(22) Filed: Mar. 27, 2025

(30) Foreign Application Priority Data
Apr. 2, 2024 (KR) 10-2024-0044490

(51) Int. Cl. A61B 1/313; A61B 1/00; A61B 34/30; A61B 34/37

(52) U.S. Cl. CPC A61B 1/313; A61B 1/00025; A61B 1/00087; A61B 34/37; A61B 2034/301; A61B 2034/306

(57) ABSTRACT

A surgical instrument is provided. Specifically, a manipulation module for a surgical instrument, and a surgical instrument including the same are provided. The manipulation module is coupled to an instrument module including an end tool having a pair of jaws formed to be rotatable, the manipulation module including a manipulation part configured to enable a user to perform a manipulation to control a motion of the end tool, and a power connection part, which is connected to one side of the manipulation part, and detachably coupled to the instrument module to transmit a driving force for controlling the motion of the end tool to the instrument module, wherein the driving force is generated based on the manipulation performed by the user.

### CROSS-REFERENCE TO RELATED APPLICATION

This application is based on and claims priority under 35 U.S.C. § 119 to Korean Patent Application No. 10-2024-0044490, filed on Apr. 2, 2024, in the Korean Intellectual Property Office, the disclosure of which is incorporated by reference herein in its entirety.

### BACKGROUND

### 1. Field

The present disclosure relates to a surgical instrument, and more particularly, to a manipulation module for a surgical instrument, and a surgical instrument including the same.

### 2. Description of the Related Art

In medical terms, surgery refers to the treatment of diseases by cutting, incising, or manipulating a skin, a mucous membrane, or other tissues by using medical devices. In particular, open surgery for incising and opening the skin of a surgical site to treat, shape, or remove an organ or the like therein causes issues such as bleeding, side effects, patient's pain, or scarring. Therefore, recently, surgery performed by forming a certain hole on a skin and inserting only a medical device, for example, a laparoscopic instrument or a surgical instrument, or surgery using a robot has been spotlighted as an alternative.

Here, a surgical robot refers to a robot that has a function of replacing a surgical action performed by a surgeon. Advantageously, the surgical robot may operate more accurately and precisely as compared with a human and enable remote surgery.

Surgical robots that are currently being developed worldwide may include a bone surgical robot, a laparoscopic surgical robot, a stereotactic surgical robot, and the like. Here, the laparoscopic surgical robot is a robot that performs minimum invasive surgery by using a laparoscope and small surgical instruments.

Laparoscopic surgery is a cutting-edge surgery technique that involves perforating one or more small holes in the abdomen and inserting a laparoscope, which is an endoscope for looking inside the abdomen to perform the surgery, and is a field that is expected to advance in the future. Today's laparoscopes are mounted with computer chips and have been developed to the extent that magnified images, which are clearer than images seen with the naked eye, may be obtained, and when used with specially-designed laparoscopic surgical tools while looking at a monitor screen, any type of surgery is possible.

Moreover, laparoscopic surgery offers the same range of surgical procedures as open surgery, but with several advantages including fewer complications, the ability to initiate treatment shortly after the procedure, and the capability to maintain the patient's stamina and immune functions. As a result, laparoscopic surgery is becoming increasingly recognized as the standard surgery for treating colorectal cancer or the like in places such as the United States and Europe.

Meanwhile, a surgical robot is generally composed of a master robot and a slave robot. When a surgical operator manipulates a control lever (e.g., a handle) provided on the master robot, a surgical tool coupled to or held by a robotic arm on the slave robot is manipulated to perform surgery.

The above-mentioned background art is technical information possessed by the inventor for the derivation of the present disclosure or acquired during the derivation of the present disclosure, and cannot necessarily be said to be a known technique disclosed to the general public prior to the filing of the present disclosure.

### SUMMARY

The present disclosure provides a manipulation module for a surgical instrument in which a manipulation part for a surgical instrument is modularized to be reusable, enabling use of the same surgical instrument as a surgical robot.

However, the above objective is an example, and the objectives of the present disclosure are not limited thereto.

Additional aspects will be set forth in part in the description which follows and, in part, will be apparent from the description, or may be learned by practice of the presented embodiments of the disclosure.

An aspect of the present disclosure provides a manipulation module to be coupled to an instrument module including an end tool having a pair of jaws formed to be rotatable, the manipulation module including a manipulation part configured to enable a user to perform a manipulation to control a motion of the end tool, and a power connection part, which is connected to one side of the manipulation part, and detachably coupled to the instrument module to transmit a driving force for controlling the motion of the end tool to the instrument module, wherein the driving force is generated based on the manipulation performed by the user.

In addition, the manipulation part may include a handle that may be held by the user.

In addition, the handle may be formed to be relatively rotatable in the left and right directions with respect to the power connection part by manipulation by the user, and when the handle rotates in the left or right direction with respect to the power connection part, the end tool may yaw-rotate.

In addition, the manipulation part may be hinge-coupled to the power connection part to be rotatable in an upward or downward direction with respect to the power connection part by a manipulation by the user, and when the manipulation part rotates in the upward or downward direction with respect to the power connection part, the end tool may pitch-rotate.

In addition, the power connection part may include a coupling area coupled to the instrument module, and the driving force may be transmitted in the coupling area.

In addition, the power connection may further include at least one power transmission member arranged to overlap the coupling area at least in part, and configured to transmit the driving force to the instrument module.

In addition, the instrument module may further include at least one driving member configured to be coupled to the surgical robot to receive power from the surgical robot, and control the motion of the end tool by the received power, and the power transmission member may be coupled to the driving member in a one-to-one correspondence.

In addition, the power transmission member may be configured to, when the manipulation part is manipulated by the user, rotate in at least one direction.

In addition, the power connection part may further include at least one power transmission pulley connected to the power transmission member to rotate together, and at least one wire that is at least partially wound around the power transmission pulley, and moves to rotate the power transmission pulley by a manipulation by the user.

In addition, the power transmission member may include a plurality of power transmission members configured to rotate on a same plane.

In addition, the power connection part may further include a jaw power transmission member coupled to the instrument module to transmit, to the instrument module, a driving force for controlling a rotational motion of the pair of jaws.

In addition, the jaw power transmission member may include a first jaw power transmission member and a second jaw power transmission member that are formed to operate independently of each other.

In addition, the power connection part may further include a pitch power transmission member coupled to the instrument module to transmit, to the instrument module, a driving force for controlling a pitch motion of the end tool.

In addition, the power transmission member may include a protrusion that protrudes outward such that at least a portion thereof is inserted into the instrument module.

In addition, the manipulation module may further include a guide hole formed such that a shaft-shaped connection part provided in the instrument module is inserted therein, and configured to guide a coupling direction of the instrument module.

In addition, the power connection part may include at least one fastening part coupled and fastened to the instrument module.

Another aspect of the present disclosure provides a surgical instrument including an instrument module including an end tool having a pair of jaws formed to be rotatable, and a driving part configured to control a motion of the end tool, and a manipulation module detachably coupled to the instrument module and configured to transmit, to the driving part, a driving force for controlling the motion of the end tool.

In addition, the manipulation module may include a manipulation part configured to enable a user to perform a manipulation to control the motion of the end tool, and a power connection part that has one side connected to the manipulation part and another side detachably connected to the driving part, and is configured to transmit, to the driving part, a driving force generated based on a manipulation by the user.

In addition, the power connection part may include a coupling area coupled to the driving part, and the driving force may be transmitted in the coupling area.

In addition, the power connection part may further include at least one power transmission member arranged to overlap the coupling area at least in part, and configured to transmit the driving force to the driving part, and the driving part may include at least one driving member coupled to the power transmission member to receive the driving force from the power transmission member.

In addition, the power transmission member may be configured to, when the manipulation part is manipulated by the user, rotate in at least one direction, and the driving member may be configured to be engaged with the power transmission member to rotate together with the power transmission member.

In addition, the power connection part may further include at least one power transmission pulley connected to the power transmission member to rotate together, and at least one wire that is at least partially wound around the power transmission pulley, and moves to rotate the power transmission pulley by a manipulation by the user.

In addition, the driving part may further include at least one pulley connected to the driving member to rotate together, and at least one wire arranged to connect the pulley and the end tool and transmit, to the end tool, a driving force for controlling the motion of the end tool, while moving by rotation of the pulley.

In addition, the power transmission member may include a plurality of power transmission members configured to rotate on a same plane, and the driving member may include a plurality of driving members configured to rotate on a same plane.

In addition, the power transmission member and the driving member may be coupled to each other in a one-to-one correspondence.

In addition, the power connection part may further include a jaw power transmission member configured to transmit, to the driving part, a driving force for controlling a rotational motion of the pair of jaws, and the driving part may further include a jaw driving member coupled to the jaw power transmission member to receive, from the jaw power transmission member, the driving force for controlling the rotational motion of the pair of jaws.

In addition, the jaw power transmission members include a first jaw power transmission member and a second jaw power transmission member formed to operate independently of each other, and the jaw driving member may include a first jaw driving member corresponding to the first jaw power transmission member, and a second jaw driving member corresponding to the second jaw power transmission member.

In addition, the power connection part may further include a pitch power transmission member configured to transmit, to the driving part, a driving force for controlling a pitch motion of the end tool, and the driving part may further include a pitch driving member coupled to the pitch power transmission member to receive, from the pitch power transmission member, the driving force for controlling the pitch motion of the end tool.

In addition, the power transmission member may include a protrusion that protrudes outward, and the driving member may include an insertion groove formed such that the protrusion is inserted therein.

In addition, the instrument module may further include a connection part formed to extend from the driving part toward the end tool, and the manipulation module may further include a guide hole formed such that the connection part is inserted therein, and configured to guide a coupling direction of the instrument module.

In addition, the manipulation module may include at least one fastening part coupled and fastened to the driving part.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is a conceptual diagram illustrating a surgical robotic system on which a surgical instrument is mounted, according to an embodiment of the present disclosure;

FIG. 1B is a perspective view illustrating a slave robot of the surgical robotic system of FIG. 1A;

FIG. 2 is a perspective view illustrating an instrument module of a surgical instrument according to an embodiment of the present disclosure;

FIG. 3 is a diagram illustrating an embodiment in which the instrument module of FIG. 2 is mounted on a manipulation module;

FIG. 4 is a diagram illustrating an example in which the instrument module of FIG. 2 is mounted on a robotic arm unit of FIG. 1B;

FIGS. 5 to 7 are diagrams illustrating an end tool for a surgical instrument according to an embodiment of the present disclosure;

FIGS. 8 to 11 are diagrams illustrating an end tool for a surgical instrument according to another embodiment of the present disclosure;

FIGS. 12A to 13 are cross-sectional views overall illustrating a stapling motion of the end tool for a surgical instrument of FIG. 8;

FIG. 14 is a perspective view of a driving part of the instrument module of FIG. 2;

FIGS. 15 and 16 are diagrams illustrating an internal structure of a driving part;

FIGS. 17 to 20 are diagrams for describing an arrangement structure of pulleys and wires inside the driving part of FIG. 14;

FIG. 21 is an enlarged view of portion X of FIG. 15;

FIG. 22 is a diagram illustrating an example in which an instrument module is coupled to a slave robot, according to an embodiment of the present disclosure;

FIG. 23 is an enlarged view of portion Y of FIG. 22;

FIG. 24 is a diagram illustrating a power transmission unit of FIG. 22;

FIG. 25 is a diagram illustrating an internal structure of the power transmission unit of FIG. 24.

FIG. 26 is a diagram for describing a connection structure in which the instrument module of FIG. 22 is mounted on a slave robot;

FIGS. 27 and 28 are diagrams illustrating a manipulation module according to an embodiment of the present disclosure;

FIG. 29 is a diagram illustrating a surgical instrument according to an embodiment of the present disclosure;

FIG. 30 is an enlarged view of portion Z of FIG. 27;

FIG. 31 is a diagram for describing a connection structure in which an instrument module of FIG. 29 is coupled to a manipulation module;

FIG. 32 is a conceptual diagram for describing an operation of the surgical instrument of FIG. 3;

FIGS. 33 to 36 are perspective views illustrating a pitch motion of the surgical instrument of FIG. 3;

FIGS. 37 to 40 are perspective views illustrating a yaw motion of the surgical instrument of FIG. 3;

FIGS. 41 to 44 are perspective views illustrating a state in which the end tool for a surgical instrument of FIG. 3 is pitch-rotated and yaw-rotated; and

FIGS. 45 to 48 are diagrams for describing an arrangement structure of pulleys and wires inside the manipulation module of FIG. 27.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

As the present disclosure allows for various changes and numerous embodiments, particular embodiments will be illustrated in the drawings and described in detail. Advantages and features of the present disclosure and a method of achieving the same should become clear with embodiments described below in detail with reference to the drawings. However, the present disclosure is not limited to the embodiments disclosed below, but may be implemented in various forms.

Hereinafter, embodiments will be described in detail with reference to the accompanying drawings, and the same or corresponding components will be denoted by the same reference numerals when described with reference to the accompanying drawings, and thus, their descriptions that are already provided will be omitted.

In the following embodiments, terms such as “first,” “second,” etc., are used only to distinguish one component from another, and such components must not be limited by these terms.

In the following embodiments, the singular expression also includes the plural meaning as long as it is not inconsistent with the context.

In the following embodiments, the terms “comprises,” “includes,” “has”, and the like used herein specify the presence of stated features or components, but do not preclude the presence or addition of one or more other features or components.

For convenience of description, the magnitude of components in the drawings may be exaggerated or reduced. For example, since the size and thickness of each component illustrated in the drawing are arbitrarily shown for convenience of description, the present disclosure is not necessarily limited to those illustrated in the drawing.

In the following embodiments, the x-axis, y-axis, and z-axis are not limited to three axes on a Cartesian coordinate system, and may be interpreted in a broad sense including them. For example, the x-axis, the y-axis, and the z-axis may be orthogonal to each other, but may refer to different directions that are not orthogonal to each other.

In a case in which a particular embodiment is realized otherwise, a particular process may be performed out of the order described. For example, two processes, which are successively described herein, may be substantially simultaneously performed, or may be performed in a process sequence opposite to a described process sequence.

Hereinafter, based on the above-described principles, a surgical instrument and a surgical robotic system including the same according to the present disclosure will be described in detail with reference to the drawings.

### <Surgical Robotic System>

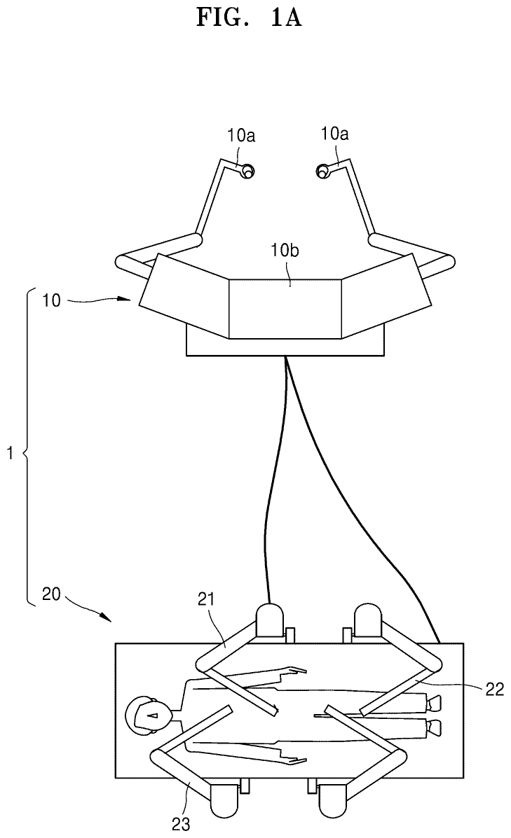

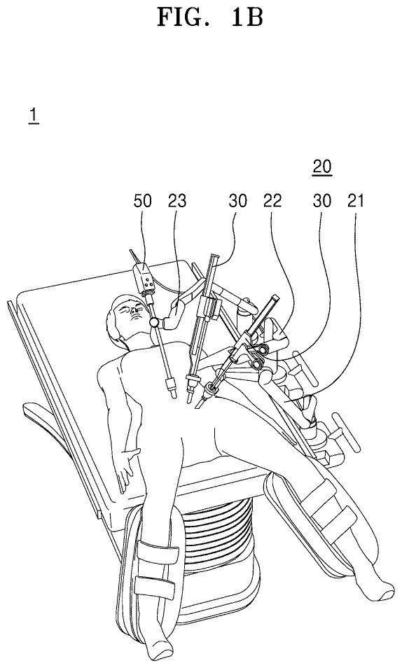

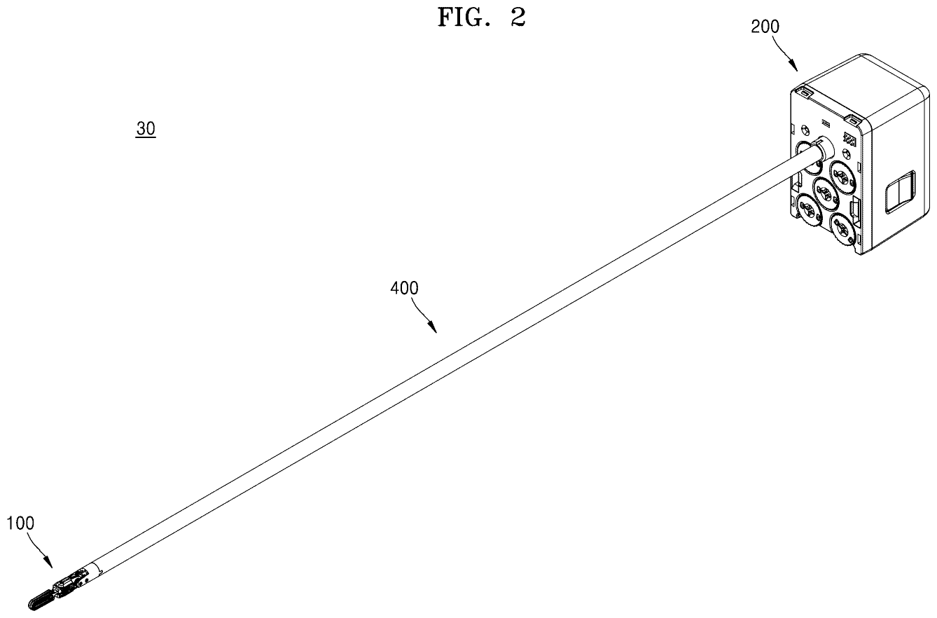

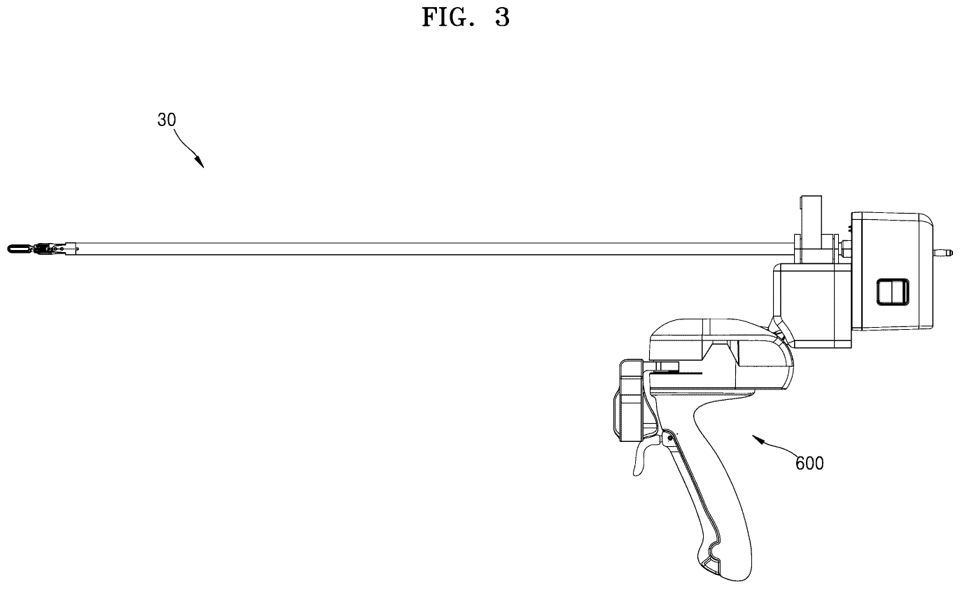

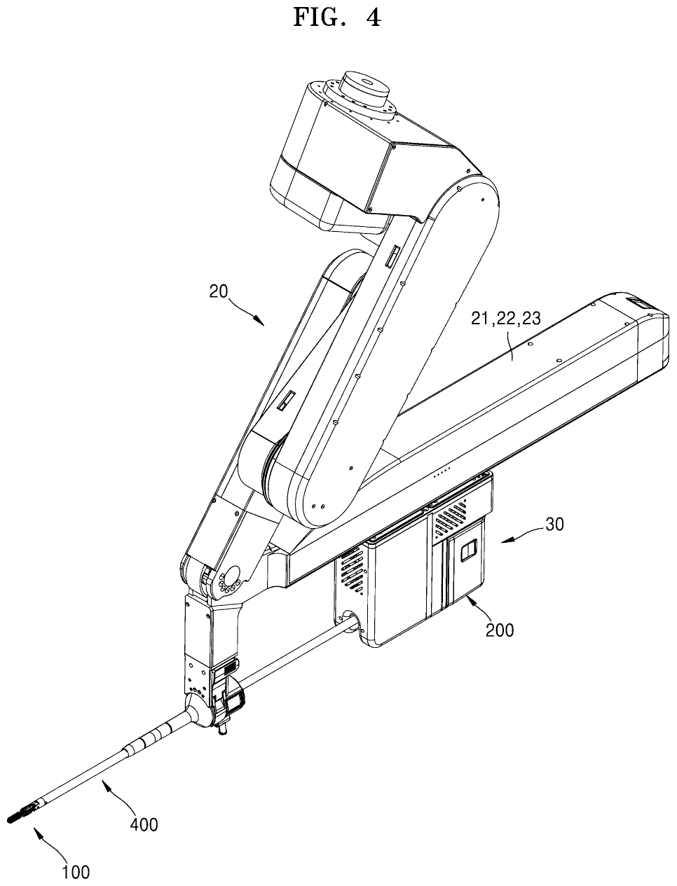

FIG. 1A is a conceptual diagram illustrating a surgical robotic system on which a surgical instrument is mounted, according to an embodiment of the present disclosure, and FIG. 1B is a perspective view illustrating a slave robot of the surgical robotic system of FIG. 1A. FIG. 2 is a perspective view illustrating an instrument module of a surgical instrument according to an embodiment of the present disclosure, FIG. 3 is a diagram illustrating an embodiment in which the instrument module of FIG. 2 is mounted on a manipulation module, and FIG. 4 is a diagram illustrating an example in which the instrument module of FIG. 2 is mounted on a robotic arm unit of FIG. 1B.

Referring to FIGS. 1 to 4, a surgical robotic system may include an instrument module 30. The instrument module 30 according to the present disclosure is a modular surgical instrument that may be selectively mounted and used on a slave robot 20 and a manipulation module 600, as will be described below.

Referring to FIGS. 1A, 1B, and 4, a surgical robotic system 1 may include a master robot 10, the slave robot 20, and the instrument module 30. For example, the instrument module 30 may be mounted and used on a surgical robot.

The master robot 10 may include a manipulation member 10a and a display member 10b, and the slave robot 20 may include one or more robotic arm units 21, 22, and 23.

In detail, the master robot 10 may include the manipulation member 10a to allow an operator to hold and manipulate the manipulation member 10a with both hands. In addition, an image captured through a laparoscope 50 may be displayed as a screen image on the display member 10b of the master robot 10. In addition, a predetermined virtual manipulation panel may be displayed on the display member 10b independently of or together with the image captured through the laparoscope 50. Detailed descriptions of the arrangement and configuration of the virtual manipulation panel will be omitted.

Meanwhile, the slave robot 20 may include one or more robotic arm units 21, 22, and 23. Here, each of the robotic arm units 21, 22, and 23 may be provided in the form of a module that may operate independently of each other, and in this case, an algorithm for preventing collisions between the robotic arm units 21, 22, and 23 may be applied to the surgical robotic system 1.

Here, the instrument module 30 may be attached to two or more of the robotic arm units 21, 22, and 23, and the laparoscope 50 may be attached to one or more of the robotic arm units 21, 22, and 23. In addition, a surgeon may select the robotic arm unit 21, 22, or 23 to be controlled through the master robot 10. As such, the surgeon may directly control a total of three or more surgical instruments through the master robot 10 without the need for a surgical assistant, and thus manipulate various instruments accurately and freely as intended by the surgeon.

Referring to FIG. 3, the surgical instrument according to an embodiment of the present disclosure may include the instrument module 30 and the manipulation module 600 for a surgical instrument (hereinafter, referred to as a manipulation module).

The manipulation module 600 may be configured as a hand-held type to be held by a user. That is, a user, such as a surgeon, may operate the instrument module 30 by manipulating the manipulation module 600 in various ways.

The manipulation module 600 may be formed to be mountable on the instrument module 30, and in more detail, the manipulation module 600 may be detachably mounted to the instrument module 30. For example, the manipulation module 600 may be configured in a forceps shape, a stick shape, a lever shape, or the like, and when the user manipulates the manipulation module 600, an end tool 100 inserted into a patient's body performs a certain motion such that surgery is performed.

Here, the instrument module 30 is mounted on the robotic arm unit 21, 22, or 23 described above. In other words, it may also be described that the manipulation module 600 according to the present disclosure is a component for allowing the user to directly manipulate the instrument module 30 mounted on the surgical robot. In other words, it may also be described that the instrument module 30 according to the present disclosure is modularized to be compatible with the surgical robot and the manipulation module 600.

In addition, although FIG. 3 and the like illustrates that the manipulation module 600 includes a handle that may be held and a ring that may be rotated with a finger inserted therein, the present disclosure is not limited thereto, and various types of manipulation modules 600 that may be connected to the end tool 100 to manipulate the end tool 100 may be possible.

As such, the instrument module 30 according to the present disclosure may be formed in a modular form. Thus, when an abnormality occurs in the master robot 10 or the slave robot 20 during surgery, or when the user needs to perform surgery in a hand-held manner, the user may separate the instrument module 30 from the robotic arm unit 21, 22, or 23, mount it on the manipulation module 600, and then continue the surgery without stopping the surgery.

Referring back to FIG. 2, the instrument module 30 according to an embodiment of the present disclosure may include the end tool 100, a driving part 200, and a connection part 400.

Here, the connection part 400 may be formed in the shape of a hollow shaft to accommodate therein one or more wires (to be described below), and the driving part 200 may be coupled to one end of the connection part 400, and the end tool 100 is coupled to the other end of the connection part 400 such that the connection part 400 serves to connect the driving part 200 to the end tool 100.

The driving part 200 is formed at one end of the connection part 400 and provides an interface that may be coupled to the robotic arm unit 21, 22, or 23, and the manipulation module 600.

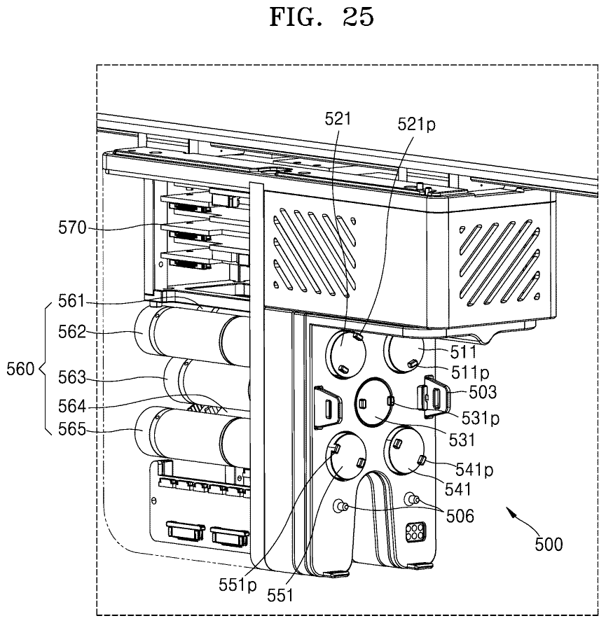

Thus, when the master robot 10 is operated by the user, a motor (see 560 of FIG. 25) of the robotic arm unit 21, 22, or 23 operate to allow the end tool 100 of the instrument module 30 to perform a corresponding motion, and a driving force of the motor (see 560 of FIG. 25) is transmitted to the end tool 100 through the driving part 200. In other words, it may also be described that the driving part 200 itself serves as an interface connecting the instrument module 30 to the slave robot 20.

In addition, when the instrument module 30 is mounted on the manipulation module 600 and then the manipulation module 600 is manipulated by the user, a plurality of wires and pulleys arranged inside the manipulation module 600 operate such that the end tool 100 of the instrument module 30 may perform a corresponding motion, and accordingly, the driving force is transmitted to the end tool 100 through the driving part 200. In other words, it may also be described that the driving part 200 itself serves as an interface connecting the instrument module 30 to the manipulation module 600.

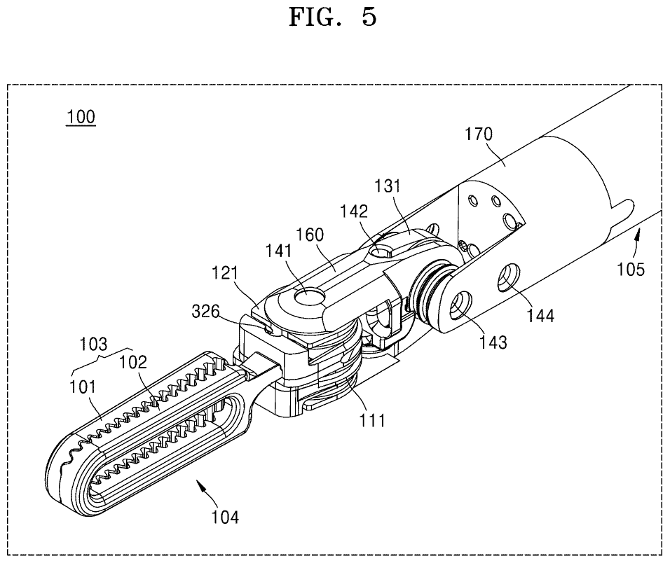

The end tool 100 may be formed at the other end of the connection part 400 and may be inserted into a surgical site to perform a motion necessary for surgery. The end tool 100 will be described in more detail below with reference to FIG. 5 and the subsequent drawings.

### <First Embodiment of End Tool for Surgical Instrument>

FIGS. 5 to 7 are diagrams illustrating an end tool for a surgical instrument according to an embodiment of the present disclosure.

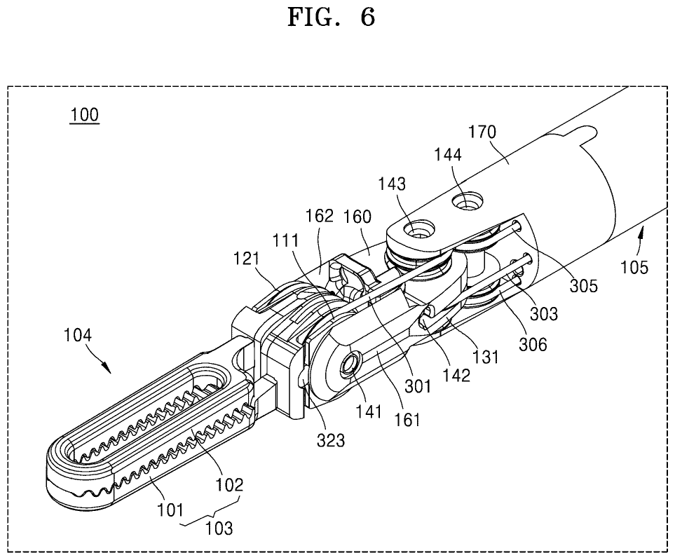

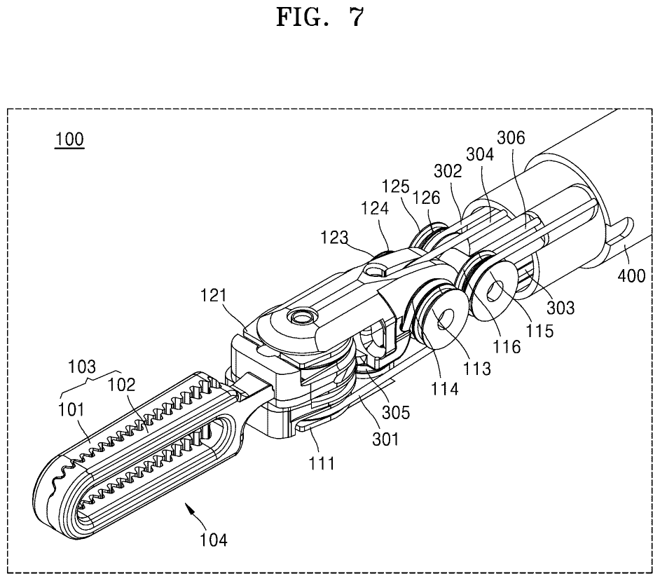

In detail, FIG. 5 is a perspective view illustrating an end tool for a surgical instrument according to an embodiment of the present disclosure, and FIG. 6 is a perspective view illustrating the end tool of FIG. 5 rotated by 90°, and FIG. 7 is a diagram illustrating the end tool of FIG. 5 from which a pitch hub is removed.

Referring to FIGS. 5 to 7, the end tool 100 for a surgical instrument (hereinafter, referred to as an end tool) according to an embodiment of the present disclosure includes a pair of jaws for performing a grip motion, that is, a first jaw 101 and a second jaw 102. Here, a component encompassing each of the first jaw 101 and the second jaw 102 or both the first jaw 101 and the second jaw 102 may be referred to as a jaw 103.

The end tool 100 may be inserted into a surgical site to perform a motion necessary for surgery. To this end, a pair of jaws 103 provided on the end tool 100 may be used.

The end tool 100 may function as a surgical clamp, grasper, vessel sealer, stapler, or cauterizer (e.g., a monopolar hook or spatula) that performs a motion necessary for surgery, such as a gripping, cutting, or suturing motion, but is not limited thereto, and any tool that is to be inserted into a patient's surgical site to perform a motion necessary for surgery may be used as the end tool 100.

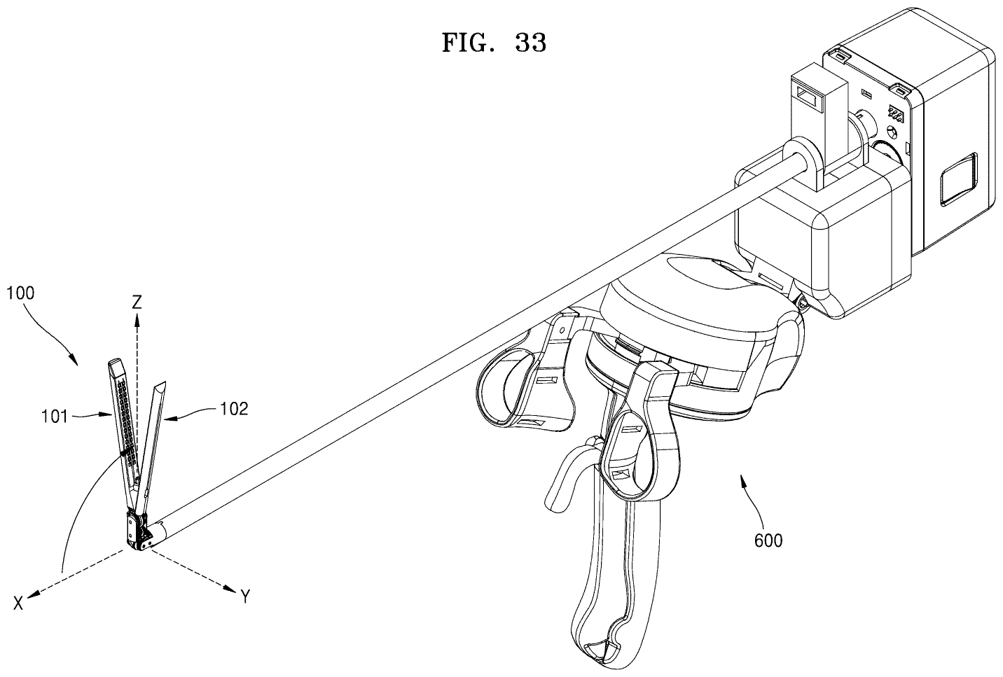

In order to perform such a function, the end tool 100 may be formed to be rotatable in two or more directions, and for example, the end tool 100 may be formed to perform a pitch motion around the Y-axis of FIGS. 33 to 44 and simultaneously perform a yaw motion and an actuation motion around the Z-axis.

Here, the pitch, yaw, roll, and actuation motions used in the present disclosure are defined as follows.

First, the pitch motion refers to a motion of the end tool 100 rotating in a vertical direction with respect to an extension direction of the connection part 400 (the X-axis direction of FIG. 33), that is, a motion of rotating around the Y-axis of FIG. 33. In other words, the pitch motion refers to a motion of the end tool 100, which extends from the connection part 400, rotating in the vertical direction around the Y-axis with respect to the connection part 400.

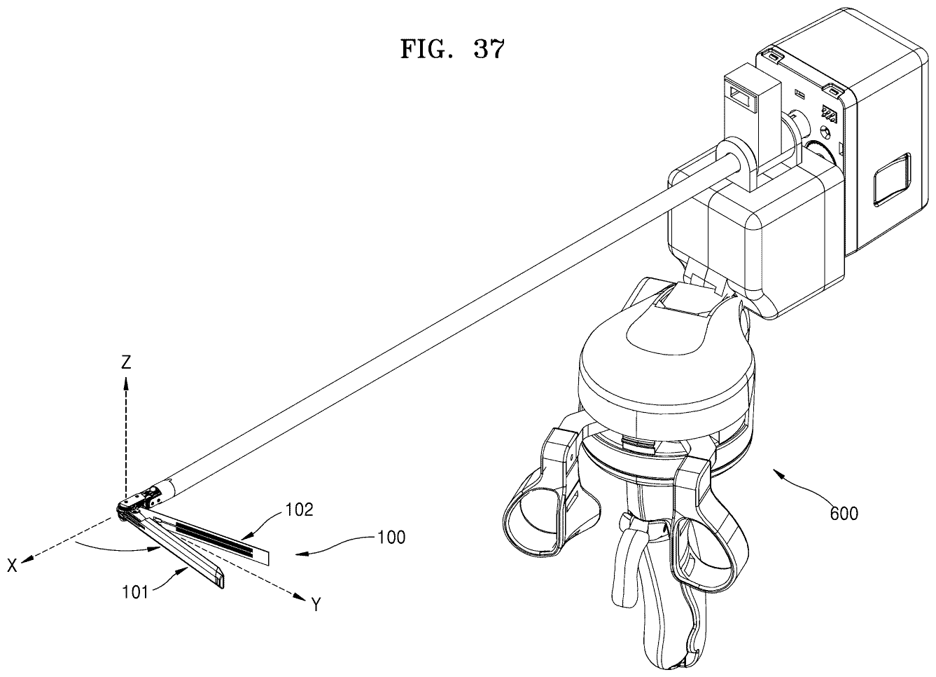

Next, the yaw motion refers to a motion of the end tool 100 rotating in a horizontal direction with respect to the extension direction of the connection part 400 (the X-axis direction of FIG. 37), that is, a motion of rotating around the Z-axis of FIG. 37. In other words, the yaw motion refers to a motion of the end tool 100, which extends from the connection part 400, rotating in the horizontal direction around the Y-axis with respect to the connection part 400. That is, the yaw motion refers to a motion of the two jaws 101 and 102, which are formed on the end tool 100, rotating around the Z-axis in the same direction.

Next, the roll motion refers to a motion of the end tool 100 and/or the connection part 400 rotating clockwise or counterclockwise around the extension direction of the connection part 400 (the X-axis direction of FIG. 37), that is, a motion of rotating around the X-axis of FIG. 37. In other words, the roll motion refers to a motion of the end tool 100, which extends from the connection part 400, rotating clockwise or counterclockwise around the X-axis independently of or together with the connection part 400. In other words, the roll motion refers to a motion of the end tool 100 and/or the connection part 400 rotating separately or together in the same direction around the X-axis.

Meanwhile, the actuation motion refers to a motion of the end tool 100 rotating around the same rotation axis as that of the yaw motion, but with the two jaws 101 and 102 rotating in the opposite directions to be closed or opened. That is, the actuation motion refers to a motion of the two jaws 101 and 102, which are formed on the end tool 100, rotating around the Z-axis in the opposite directions.

In other words, yaw rotation may refer to a motion of a jaw pulley, which will be described below, rotating around a rotation shaft 141, which is a jaw pulley rotation shaft, and pitch rotation may refer to a motion of the jaw pulley revolving around a rotation shaft 143, which is a pitch main rotation shaft.

The end tool 100 may include a pulley 111, a pulley 113, a pulley 114, a pulley 115, and a pulley 116, which are associated with a rotational motion of the first jaw 101. In addition, the end tool 100 may include a pulley 121, a pulley 123, a pulley 124, a pulley 125, and a pulley 126, which are associated with a rotational motion of the second jaw 102.

Here, although the drawings illustrate that the pulleys facing each other are arranged in parallel to each other, the present disclosure is not limited thereto, and the pulleys may be formed in various positions and sizes suitable for the configuration of the end tool.

In addition, the end tool 100 may include therein a wire 301, a wire 302, a wire 303, a wire 304, a wire 305, and a wire 306, which implement a motion of the end tool 100. That is, the wire 301, the wire 302, the wire 303, the wire 304, the wire 305, and the wire 306 may be wound around the pulley 111, the pulley 113, the pulley 114, the pulley 115 and the pulley 116, which are associated with the rotational motion of the first jaw 101, and the pulley 121, the pulley 123, the pulley 124, the pulley 125, and pulley 126, which are associated with the rotational motion of the second jaw 102, and may transmit a driving force received from the outside, to the first jaw 101 and the second jaw 102.

Here, the wire 301 and the wire 305 may be paired to serve as a first jaw wire. The wire 302 and the wire 306 may be paired to serve as a second jaw wire. Here, a component encompassing the wire 301 and the wire 305, which constitute the first jaw wire, and the wire 302 and the wire 306, which constitute the second jaw wire, may be referred to as a jaw wire. In addition, the wire 303 and the wire 304 may be paired to serve as a pitch wire.

In addition, the end tool 100 according to an embodiment of the present disclosure may include a fastening member 323 and a fastening member 326, which are coupled to respective ends of the wires to couple the wires to the pulleys. Here, each of the fastening members may have various shapes as necessary, such as a ball shape or a tube shape.

Here, the fastening member 323 may function as a first jaw wire-end tool fastening member, and the fastening member 326 may function as a second jaw wire-end tool fastening member. However, although not illustrated in the drawings, a fastening member for respectively coupling the wires to the pulleys may be further provided.

The coupling relationship between the wires, the fastening members, and the pulleys will be described as follows.

First, the wire 301 and the wire 305, which constitute the first jaw wire, may be a single wire. When the fastening member 323, which is a second jaw wire-end tool fastening member, is fit into a middle point of the first jaw wire, and the fastening member 323 is fixed through crimping, both strands of the first jaw wire around the fastening member 323 may be referred to as the wire 301 and the wire 305, respectively.

Alternatively, the wire 301 and the wire 305, which constitute the first jaw wire, may be formed as separate wires and connected to each other by the fastening member 323.

In addition, by coupling the fastening member 323 to a pulley 111, the wire 301 and the wire 305 may be fixedly coupled to the pulley 111. Accordingly, the pulley 111 may be rotated as the wire 301 and the wire 305 are pulled and released.

In addition, a first jaw wire-manipulation part fastening member (not shown) may be coupled to ends of the wire 301 and the wire 305 that are opposite to the ends thereof to which the fastening member 323 is fastened.

Meanwhile, the wire 301 and the wire 305 may extend to the driving part 200 through the connection part 400 as will be described below. The wire 301 and the wire 305 extending to the driving part 200 may be wound around a pulley associated with a rotational motion of the first jaw 101. Here, as in the end tool 100, the wire 301 and the wire 305 may be fixed to a pulley associated with a rotational motion of the first jaw 101 by a separate fastening member (not shown).

In the same manner, the wire 302 and the wire 306, which constitute the second jaw wire, may be coupled to the fastening member 326, which is a second jaw wire-end tool fastening member. In addition, the fastening member 326 may be coupled to the pulley 121. In addition, the wire 302 and the wire 306 may extend to the driving part 200 through the connection part 400 as will be described below. The wire 302 and the wire 306 extending to the driving part 200 may be wound around a pulley associated with a rotational motion of the second jaw 102. Here, as in the end tool 100, the wire 302 and the wire 306 may be fixed to a pulley associated with a rotational motion of the second jaw 102 by a separate fastening member (not shown). Accordingly, when the pulleys associated with a rotational motion of the second jaw 102 are rotated by a motor or a human force, the pulley 121 of the end tool 100 may be rotated as the wire 302 and the wire 306 are pulled and released.

In the same manner, the wire 303 and the wire 304, which are pitch wires, may be coupled to a separately provided fastening member (not shown). In addition, the fastening member may be coupled to a pulley 131. In addition, the wire 303 and the wire 304 may extend to the driving part 200 through the connection part 400 as will be described below. The wire 303 and the wire 304 extending to the driving part 200 may be wound around a pulley associated with a pitch motion of the end tool 100. Here, as in the end tool 100, the wire 303 and the wire 304 may be fixed to a pulley associated with a pitch motion of the end tool 100 by a separate fastening member (not shown). Accordingly, when the pulleys associated with a pitch motion of the end tool 100 are rotated by a motor or a human force, the pulley 131 of the end tool 100 may be rotated as the wire 303 and the wire 304 are pulled and released.

Accordingly, the wire 301 and the wire 305, which are both strands of the first jaw wire, may be coupled to the fastening member 323, which is a first jaw wire-end tool fastening member, and the fastening member (not shown), which is a first jaw wire-driving part fastening member, so as to function as a closed loop together. Similarly, each of the second jaw wire and the pitch wire may be formed to function as a closed loop.

In addition, the end tool 100 of the present disclosure may include an end tool hub 160 and a pitch hub 170.

The rotation shaft 141 and a rotation shaft 142, which will be described below, may be inserted through the end tool hub 160, and the end tool hub 160 may accommodate therein at least portions of the first jaw 101 and the second jaw 102, which are axially coupled to the rotation shaft 141.

In addition, the pulley 131 serving as an end tool pitch pulley may be formed at one end of the end tool hub 160. Here, the pulley 131 may be formed with the end tool hub 160 as one body. That is, one end of the end tool hub 160 may be formed in a disk shape or a semicircular shape, and a groove around which a wire may be wound may be formed on an outer circumferential surface of the groove, such that a kind of guide channel is formed. Alternatively, the pulley 131 may be formed as a separate member from the end tool hub 160 and then coupled to the end tool hub 160. The wire 303 and the wire 304 described above is coupled to the pulley 131 serving as an end tool pitch pulley, and a pitch motion is performed as the pulley 131 rotates around the rotation shaft 143.

The rotation shaft 143 and a rotation shaft 144, which will be described below, may be inserted through the pitch hub 170, and the pitch hub 170 may be axially coupled to the end tool hub 160 and the pulley 131 by the rotation shaft 143. Thus, the end tool hub 160 and the pulley 131 (coupled to the end tool hub 160) may be formed to be rotatable around the rotation shaft 143 with respect to the pitch hub 170.

In addition, the pitch hub 170 may accommodate therein at least portions of the pulley 113, the pulley 114, the pulley 123, and the pulley 124, which are axially coupled to the rotation shaft 143. In addition, the pitch hub 170 may accommodate therein at least portions of the pulley 115, the pulley 116, the pulley 125, and the pulley 126, which are axially coupled to the rotation shaft 144.

In addition, the end tool 100 of the present disclosure may include the rotation shaft 141, the rotation shaft 142, the rotation shaft 143, and the rotation shaft 144. As described above, the rotation shaft 141 and the rotation shaft 142 may be inserted through the end tool hub 160, and the rotation shaft 143 and the rotation shaft 144 may be inserted through the pitch hub 170.

The rotation shaft 141, the rotation shaft 142, the rotation shaft 143, and the rotation shaft 144 may be arranged sequentially from a distal end 104 of the end tool 100 toward a proximal end 105. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Here, the rotation shaft 141 may be arranged to pass through the first jaw 101 and the second jaw 102. For example, the rotation shaft 141 may be arranged to pass through the first jaw 101 and the second jaw 102, which are at least partially accommodated in the end tool hub 160. That is, the rotation shaft 141 may be arranged to sequentially pass through a first jaw pulley coupling part 161, the pulley 111, the first jaw 101, the second jaw 102, the pulley 121, and a second jaw pulley coupling part 162 of the end tool hub 160.

One or more pulleys may be fit into each of the rotation shafts 141, 142, 143, and 144, which will be described in detail below.

The pulley 111 functions as an end tool first jaw pulley, the pulley 121 functions as an end tool second jaw pulley, and these two components may be collectively referred to as an end tool jaw pulley.

The pulley 111 and the pulley 121, which constitute the end tool jaw pulley, are formed to face each other, and are formed to be rotatable independently of each other around the rotation shaft 141, which is an end tool jaw pulley rotation shaft.

The drawings illustrate that the pulley 111 and the pulley 121 are formed to be rotated around one rotation shaft 141, but it is needless to say that each jaw pulley may be formed to be rotatable around a separate shaft. Here, the first jaw 101 may be fixedly coupled to the pulley 111 to rotate together with the pulley 111, and the second jaw 102 may be fixedly coupled to the pulley 121 to rotate together with the pulley 121. A yaw motion and an actuation motion of the end tool 100 are performed according to rotation of the pulley 111 and the pulley 121. That is, when the pulley 111 and the pulley 121 rotate in the same direction around the rotation shaft 141, the yaw motion is performed, and when the pulley 111 and the pulley 121 rotate in opposite directions around the rotation shaft 141, the actuation motion is performed.

Here, the first jaw 101 and the pulley 111 may be formed as separate members and coupled to each other, or the first jaw 101 and the pulley 111 may be formed as one body. Similarly, the second jaw 102 and the pulley 121 may be formed as separate members and coupled to each other, or the second jaw 102 and the pulley 121 may be formed as one body.

Here, the pulley 111 having formed thereon a groove around which the wire 301/wire 305, which constitute the first jaw wire, are wound, is arranged adjacent to the first jaw pulley coupling part 161 of the end tool hub 160, and the pulley 121 having formed thereon a groove around which the wire 302/wire 306, which constitute the second jaw wire, is wound, is arranged adjacent to the second jaw pulley coupling part 162 of the end tool hub 160. Thus, a certain space may be formed between the wire 301/wire 305, which constitute the first jaw wire, and the wire 302 and the wire 306, which constitute the second jaw wire. As such, as the wire 301/wire 305, which constitute the first jaw wire, and the wire 302 and the wire 306, which constitute the second jaw wire, are arranged to be spaced apart from each other, the wires may be wound around the respective pulleys while maintaining a straight line.

Meanwhile, a guide part that guides the paths of the wire 302 and the wire 306 may be formed in the end tool hub 160.

The pulley 113 and the pulley 114 may function as end tool first jaw pitch main pulleys, the pulley 123 and the pulley 124 may function as end tool second jaw pitch main pulleys, and these two components may collectively be referred to as an end tool jaw pitch main pulley. Here, the rotation shaft 143 may be arranged to pass through the pulley 113, the pulley 114, the pulley 123, and the pulley 124.

The pulley 115 and the pulley 116 may function as end tool first jaw pitch sub-pulleys, the pulley 125 and the pulley 126 may function as end tool second jaw pitch sub-pulleys, and these two components may collectively be referred to as an end tool jaw pitch sub-pulley. Here, the rotation shaft 144 may be arranged to pass through the pulley 115, the pulley 116, the pulley 125, and the pulley 126.

In addition, the pulley 113, the pulley 114, the pulley 123, the pulley 124, the pulley 115, the pulley 116, the pulley 125, and the pulley 126 may be collectively referred to as an end tool jaw pitch pulley.

Accordingly, the rotation shaft 141, the rotation shaft 142, the rotation shaft 143, and the rotation shaft 144 may be sequentially arranged from the distal end 104 of the end tool 100 toward the proximal end 105.

In addition, the pulley 111, the pulley 113/pulley 114, and the pulley 115/pulley 116, which are associated with rotation of the first jaw 101, may be sequentially arranged from the distal end 104 of the end tool 100 toward the proximal end 105.

In addition, the pulley 121, the pulley 123/pulley 124, and the pulley 125/pulley 126, which are associated with rotation of the second jaw 102, may be sequentially arranged from the distal end 104 of the end tool 100 toward the proximal end 105.

Hereinafter, components associated with rotation of the pulley 111 will be described. The pulley 113 and the pulley 114 may function as end tool first jaw pitch main pulleys. That is, the pulley 113 and the pulley 114 function as main rotation pulleys for a pitch motion of the first jaw 101. Here, the wire 301, which constitutes the first jaw wire, is wound around the pulley 113, and the wire 305, which constitutes the first jaw wire, is wound around the pulley 114.

The pulley 115 and the pulley 116 function as end tool first jaw sub-pulleys. That is, the pulley 115 and the pulley 116 function as sub-rotation pulleys for a pitch motion of the first jaw 101. Here, the wire 301, which constitutes the first jaw wire, is wound around the pulley 115, and the wire 305, which constitutes the first jaw wire, is wound around the pulley 116.

Here, the pulley 113 and the pulley 114 are arranged on one side of the pulley 111 to face each other. Here, the pulley 113 and the pulley 114 are formed to be rotatable independently of each other around the rotation shaft 143, which is an end tool pitch rotation shaft. In addition, the pulley 115 and the pulley 116 are arranged on one sides of the pulley 113 and the pulley 114, respectively, to face each other. Here, the pulley 115 and the pulley 116 are formed to be rotatable independently of each other around the rotation shaft 144, which is an end tool pitch auxiliary rotation shaft. Here, the drawings illustrate that the pulley 113, the pulley 114, the pulley 115, and the pulley 116 are formed to be rotatable around the same axis direction, but the present disclosure is not limited thereto, and the rotation axes of the respective pulleys may be formed in various directions according to their configurations.

The wire 301, which constitutes the first jaw wire, is wound sequentially around the pulley 115, the pulley 113, and the pulley 111 such that at least portions of the wire 301 come into contact with the pulleys. In addition, the wire 305 connected to the wire 301 by a fastening member is wound sequentially around the pulley 111, the pulley 114, and the pulley 116 such that at least portions of the wire 305 come into contact with the pulleys.

In other words, the wire 301 and the wire 305, which constitute the first jaw wire, are wound sequentially around the pulley 115, the pulley 113, the pulley 111, the pulley 114, and the pulley 116 such that at least portions of the wire 301 and the wire 305 come into contact with the pulleys, and are formed to move along the pulleys while rotating the pulleys.

Similarly, the wire 306, which constitutes the second jaw wire, is wound sequentially around the pulley 125, the pulley 123, and the pulley 121 such that at least portions of the wire 306 come into contact with the pulleys. In addition, the wire 302 connected to the wire 306 by a fastening member is wound sequentially around the pulley 121, the pulley 124, and the pulley 126 such that at least portions of the wire 302 come into contact with the pulleys.

In other words, the wire 306 and the wire 302, which constitute the second jaw wire, are wound sequentially around the pulley 125, the pulley 123, the pulley 121, the pulley 124, and the pulley 126 such that at least portions of the wire 306 and the wire 302 come into contact with the pulleys, and are formed to move along the pulleys while rotating the pulleys.

The pitch hub 170 is arranged at a rearmost portion of the end tool 100 and may be arranged adjacent to the connection part 400.

The pitch hub 170 may be formed in an approximately “C” shape. Here, the “C”-shaped pitch hub 170 may be coupled to the “C”-shaped end tool hub 160 to be perpendicular to each other. In other words, the end tool hub 160 and the pitch hub 170 may be coupled to each other such that the rotation shaft 141 passing through the end tool hub 160 and the rotation shaft 143 passing through the pitch hub 170 are perpendicular to each other. In detail, referring back to FIG. 5, because the pitch hub 170 is coupled to the end tool hub 160 in a state in which the pitch hub 170 is rotated by 90° around the lengthwise direction of the jaw compared to the end tool hub 160, a portion of the end tool hub 160 may be inserted into a “C”-shaped space formed in the pitch hub 170. Thus, the rotation shaft 143 may be inserted into a through hole formed in the end tool hub 160, and as the end tool hub 160 rotates as a whole around the rotation shaft 143, a pitch motion of the end tool 100 may be implemented.

The pulleys 113, 114, 115, 116, 123, 124, 125, and 126 may be accommodated in the “C”-shaped space formed in the pitch hub 170. In addition, a through hole is formed in the pitch hub 170, and thus, the rotation shaft 143 and the rotation shaft 144 may be inserted into the through hole.

The rotation shaft 143 may pass through the pitch hub 170, the pulley 113, the pulley 114, the pulley 123, and the pulley 124 to axially couple them. In addition, the rotation shaft 144 may pass through the pitch hub 170, the pulley 115, the pulley 116, the pulley 125, and the pulley 126 to axially couple them.

### Second Embodiment of End Tool for Surgical Instrument

Hereinafter, another embodiment of an end tool that may be used in the present disclosure will be described. For convenience of description, differences from the descriptions of the first embodiment of the end tool will be mainly described. Unless otherwise specified, the end tool according to the present embodiment may include the same configuration as the configuration described in the first embodiment, or may include a configuration that may be appropriately modified by a person skilled in the art to which the present disclosure pertains.

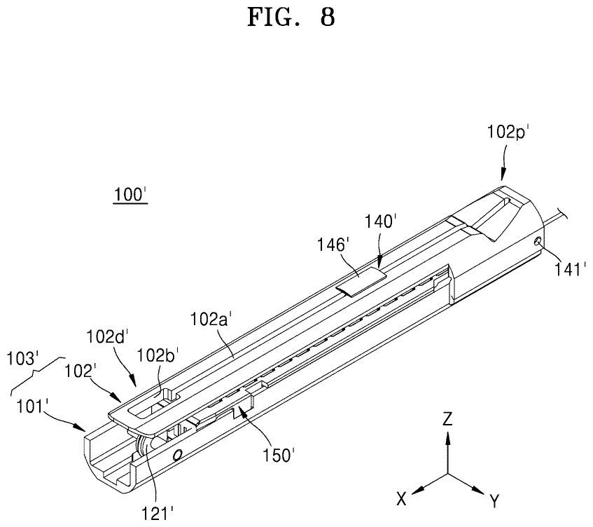

FIGS. 8 to 10 are diagrams illustrating an end tool for a surgical instrument according to another embodiment of the present disclosure.

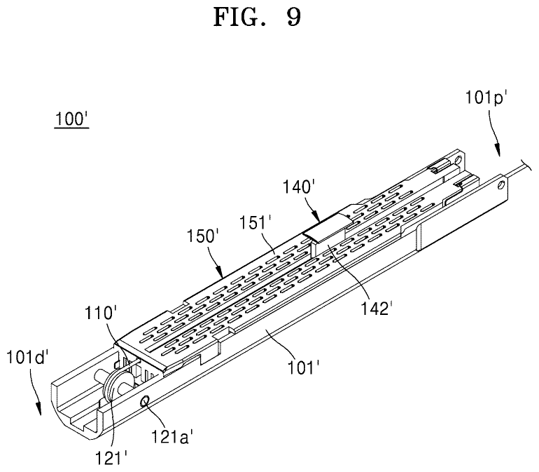

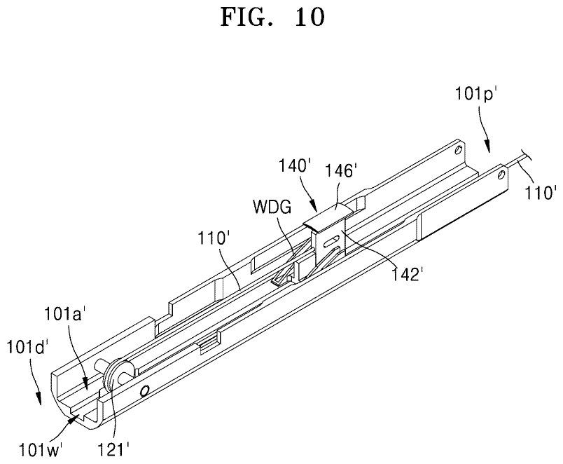

In detail, FIG. 8 is a schematic perspective view for describing an end tool according to another embodiment of the present disclosure, FIG. 9 is a schematic perspective view of the end tool of FIG. 8 from which a second jaw is removed, and FIG. 10 is a schematic perspective view of the end tool of FIG. 8 from which a cartridge is removed.

An end tool 100′ may include a jaw 103′, a fixed pulley 121′, and a forward wire 110′.

The jaw 103′ may perform various functions, for example, a grip motion, and as a specific example, may include a pair of jaws, that is, a first jaw 101′ and a second jaw 102′. Here, a component encompassing each of the first jaw 101′ and the second jaw 102′ or both the first jaw 101′ and the second jaw 102′ may be referred to as a jaw 103′.

The first jaw 101′ and the second jaw 102′ may be arranged to face each other, may move toward each other and away from each other, and for example, may be formed to rotate around one shaft 141′.

A cartridge 150′ may be arranged to be accommodated in the first jaw 101′, and a plurality of staples are arranged inside the cartridge 150′. In a state where the first jaw 101′ and the second jaw 102′ are close to each other, for example, in a state where the first jaw 101′ and the second jaw 102′ are closed to each other with body tissue interposed therebetween, when a working member 140′ receives a force from the forward wire 110′, the working member 140′ may move toward a distal end 101d′ of the first jaw 101′ to push up a staple such that stapling is performed. At this time, one or more clamps 146′ of the working member 140′, which protrude to the outside of the first jaw 101′ or the second jaw 102′, may move forward while applying pressure to an outer surface of the first jaw 101′ or the second jaw 102′ (the upper surface of the second jaw in FIG. 8), such that a stapling process is seamlessly performed.

Meanwhile, the working member 140′ may be used with a wedge WDG. For example, the wedge WDG may be prepared separately from the working member 140′ and then arranged adjacent to the working member 140′ in the first jaw 101′. In addition, as another example, the working member 140′ and the wedge WDG may be formed as one body. The wedge WDG may be formed to have a certain inclined surface. That is, the wedge WDG may be formed to be inclined to a certain degree in the extension direction of the end tool 100′. In other words, the wedge WDG may be formed such that the height of the wedge WDG on the side of a proximal end 101p′ of the first jaw 101′ is higher than the height of the wedge WDG on the side of the distal end 101d′.

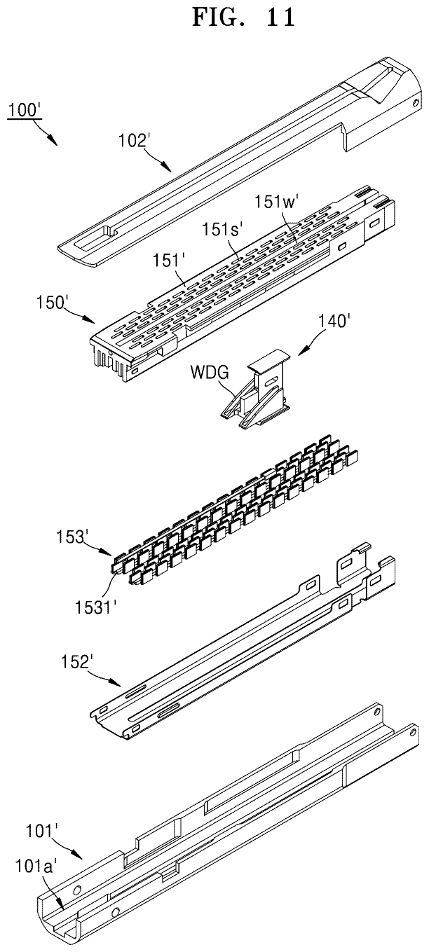

The wedge WDG may be formed to sequentially come into contact with a release member 1531′ (see FIG. 11) or a plurality of staples 153′ (see FIG. 11) arranged in the cartridge 150′, so as to serve to sequentially push up the staples 153′.

The second jaw 102′ is formed overall in an elongated bar shape, and for example, the second jaw 102′ may be formed in a bar shape to correspond to the first jaw 101′ at least in one area. An anvil may be formed on the side of a distal end 102d′ of the second jaw 102′, and the second jaw 102′ may include, on the side of the proximal end 102p′, an area that is coupled to the first jaw 101′. For example, the second jaw 102′ is formed to be rotatable around one shaft 141′ of the proximal end 102p′ with respect to the first jaw 101′.

As a specific example, an anvil may be formed on a surface of the second jaw 102′ facing the first jaw 101′, in a flat plane shape, and shapes corresponding to the shape of the staple 153′, which will be described below, may be formed on the surface. The anvil of the second jaw 102′ may serve as a support to support the opposite side of the working member 140′ when the working member 140′ pushes up the staple 153′ during a stapling motion such that the staple 153′ is bent.

The second jaw 102′ includes a guide groove 102a′. The guide groove 102a′ may have a shape elongating in the lengthwise direction of the second jaw 102′.

The guide groove 102a′ may be formed to guide the working member 140′, and may be a groove passing through an area facing the working member 140′. Through this, one area of the working member 140′ may pass through the guide groove 102a′ to be discharged to the outside of the second jaw 102′. When the working member 140′ moves forward, at least a portion of the second jaw 102′ may pass through the outside of the guide groove 102a′ to be exposed to the outside of the second jaw 102′, and come into contact with or apply pressure to the upper surface of the second jaw 102′. A portion of the working member 140′ that is exposed to the outside of the second jaw 102′ as the working member 140′ moves, that is, one side of the working member 140′, applies pressure to the upper surface of the second jaw 102′, and the other side of the working member 140′ is exposed to the outside of the first jaw 101′ to apply pressure to the lower surface of the first jaw 101′, such that the gap between the second jaw 102′ and the first jaw 101′ becomes narrower, and thus, the second jaw 102′ naturally maintains a closed state with respect to the first jaw 101′.

In an alternative embodiment, the second jaw 102′ may include a window 102b′. After working of the working member 140′ or use of the end tool 100′, the portion of the working member 140′ exposed to the outside of the second jaw 102′ may correspond to the window 102b′, and may release the coupling state between the second jaw 102′ and the working member 140′.

The first jaw 101′ is formed overall in an elongated bar shape, the cartridge 150′ is accommodated in the first jaw 101′ on the side of the distal end 101d′, and a rotation shaft may be arranged in the proximal end to enable a rotational motion, and may correspond to the rotation shaft 141′ formed in the second jaw 102′ described above.

The first jaw 101′ may be formed overall in the shape of a hollow box with one surface (the upper surface) removed, such that a cartridge accommodation part 101a for accommodating the cartridge 150′ may be formed inside the first jaw 101′. That is, the first jaw 101 may be formed in an approximately “U” shape in cross section.

A guide groove 101′h may be formed on the bottom surface of the first jaw 101′, that is, on the bottom surface facing the upper open area. In detail, the guide groove 101′h may be formed to guide a linear motion of the working member 140′.

The guide groove 101′h may be formed to guide the working member 140′, and may be a groove passing through an area facing the working member 140′. Through this, one area of the working member 140′, for example, a lower area of the working member 140′, may pass through the guide groove 101′h to be discharged to the outside of the first jaw 101′. When the working member 140′ moves forward, the portion discharged to the outside of the first jaw 101′ may pass through the guide groove 101′h of the first jaw 101′ to be exposed to the outside, and may come into contact with or apply pressure to the lower surface of the first jaw 101′. A portion of the working member 140′ that is exposed to the outside of the first jaw 101′ as the working member 140′ moves, that is, one side of the working member 140′, applies pressure to the lower surface of the first jaw 101′, and the other side of the working member 140′ applied pressure to the upper surface of the second jaw 102′, such that the gap between the second jaw 102′ and the first jaw 101′ becomes narrower, and thus, the second jaw 102′ naturally maintains a closed state with respect to the first jaw 101′.

In an alternative embodiment, the first jaw 101′ may include a window 101′b. After working of the working member 140′ or use of the end tool 100′, the portion of the working member 140′ exposed to the outside of the first jaw 101′ corresponds to the window 101′b, and may release the coupling state between the first jaw 101′ and the working member 140′.

The first jaw 101′ may include an extension groove 101w′ on the bottom surface thereof. The extension groove 101w′ may be formed on the bottom surface of the first jaw 101′, that is, on the bottom of a surface where a cartridge accommodation part 101a′ for accommodating the cartridge is formed.

The extension groove 101w′ may be formed to overlap the movement path of the working member 140′, may have a predetermined length in the lengthwise direction of the first jaw 101′, and for example, may be formed to have the same length as the length of the first jaw 101′.

In an alternative embodiment, the extension groove 101w′ may be formed to overlap the guide groove 101′h. For example, the extension groove 101w′ may be formed on the bottom surface of the surface where the accommodation part 101a′ of the first jaw 101′ is formed, with one area removed, and the guide groove 101′h that overlaps the extension groove 101w′ and has a width less than or equal to the width of the extension groove 101w′ may be formed to pass through the bottom surface of the first jaw 101′.

Through this, the arrangement and movement of the forward wire 110′ and the arrangement and movement of the working member 140′ may be efficiently controlled.

Hereinafter, the forward wire 110′ and the fixed pulley 121′ that drive the working member 140′ will be described.

The fixed pulley 121′ and the forward wire 110′ may be arranged to move the working member 140′.

The fixed pulley 121′ may be arranged on the first jaw 101′, for example, may be connected to a pulley shaft 121a′ in a direction crossing the widthwise direction of the first jaw 101′, and may be coupled to or integrally formed with the pulley shaft 121a′. In an alternative embodiment, the fixed pulley 121 may be formed separately from the pulley shaft 121a′ to rotate around the pulley shaft 121a′.

The forward wire 110′ may be wound around the outer circumferential surface of the fixed pulley 121′, and to this end, a groove may be formed on the outer circumferential surface of the fixed pulley 121′.

The fixed pulley 121′ may be arranged closer to the distal end 101d′ of the first jaw 101′ than at least the working member 140′.

The fixed pulley 121′ may have a vertically arranged form, for example, may be arranged parallel to the direction from the first jaw 101′ to the second jaw 102′, and may be arranged to correspond to the center line of the first jaw 101′.

At least one area of the fixed pulley 121′, for example, a lower area, may be arranged to correspond to the extension groove 101w′ of the first jaw 101′.

Because the forward wire 110′ is wound in correspondence to the fixed pulley 121′, one area of the forward wire 110′ may also be arranged to correspond to the extension groove 101w′ of the first jaw 101′.

Through this, the forward wire 110′ and the fixed pulley 121′ may be efficiently arranged while making it easy to secure a space for movement and stapling of the working member 140′ of the first jaw 101′.

The forward wire 110′ may extend to have a length in the lengthwise direction of the first jaw 101′, and an area at one end of the forward wire 110′ may extend to the proximal end 101p′ of the first jaw 101′ to be connected to the inside of the driving part and thus pulled by a driving force transmitted from the outside.

The other end of the forward wire 110′ may extend toward the distal end 101d′ of the first jaw 101′ in the lengthwise direction of the first jaw 101′, to pass through a wire passage part of the working member 140′, then be wound around the lower side of the fixed pulley 121′, then come out from the upper side, and then extend toward the proximal end 101p′ of the first jaw 101′ to be connected and fixed to a connection area of the working member 140′.

In an alternative embodiment, the area of the forward wire 110′, which extends to the distal end 101d′ of the first jaw 101′, passes through the wire passage part of the working member 140′, and moves toward the lower side of the fixed pulley 121′, may come out from the upper side of the fixed pulley 121′ and extend toward the proximal end 101p′ of the first jaw 101′, to be parallel to the area facing the connection area of the working member 140′.

Through this, when the forward wire 110′ is pulled toward the proximal end 101p′, the pulling force may be effectively transmitted to the working member 140′, and the pulling distance and the forward movement distance of the working member 140′ may be controlled to be almost similar or equal to each other. In addition, for example, the ratio of the forward movement distance of the working member 140′, which corresponds to the pulling force when the forward wire 110′ is pulled toward the proximal end 101p′, may be precisely controlled at a predetermined ratio, and as a specific example, the ratio of the pulling force when pulling the forward wire 110′ toward the proximal end 101p′ to the forward movement distance of the working member 140′ may be easily controlled to be a value equal to, substantially equal to, or similar to 1 to 1.

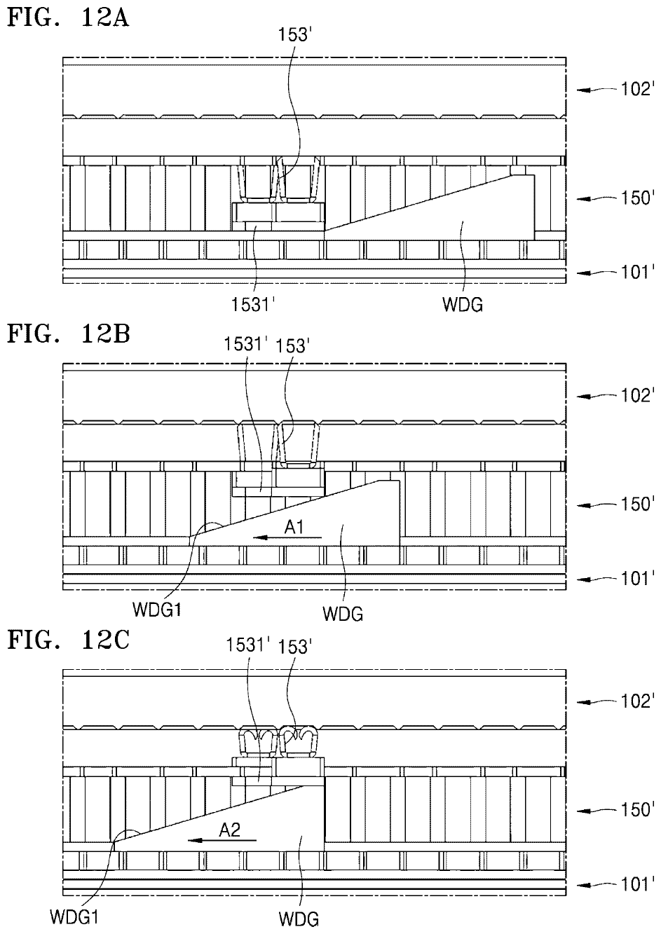

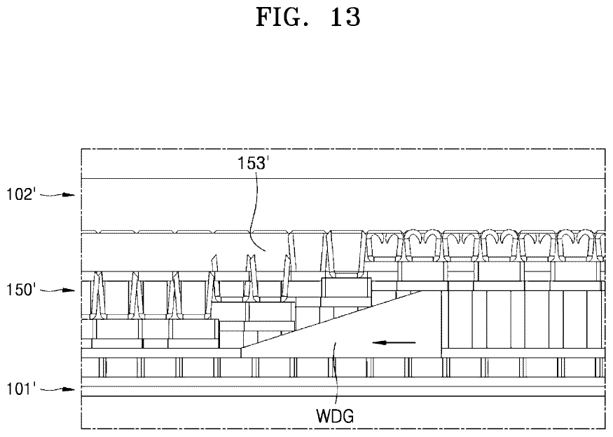

FIG. 11 is an exploded perspective view of the first jaw 101′, the second jaw 102′, and the cartridge 150′ of FIG. 8, and FIGS. 12A to 13 are cross-sectional views overall illustrating a stapling motion of the end tool for a surgical instrument of FIG. 8.

Referring to FIGS. 11 to 13, the cartridge 150′ may be arranged in the first jaw 101′, and for example, the cartridge 150′ may be arranged to be coupled to the cartridge accommodation part 101a′ of the first jaw 101′. For example, the cartridge 150′ may be formed integrally with the first jaw 101′ in a state where the working member 140′ is arranged in the first jaw 101′. In addition, in an alternative embodiment, the cartridge 150′ may be formed to be attachable to and detachable from the first jaw 101′.

The cartridge 150′ includes a plurality of staples 153′ therein to perform tissue suturing, and performs cutting through the working member 140′. Here, the cartridge 150′ may include a cover 151′, the staples 153′, and the release member 1531′.

The cover 151′ may be formed to cover an upper portion of the cartridge accommodation part 101a′ of the first jaw 101′. Staple holes 151s′ through which a plurality of staples 153′ may be ejected to the outside may be formed in the cover 151′. Stapling may be performed as the staples 153′, which are accommodated in the cartridge accommodation part 101a′ before a stapling operation, are pushed up by the working member 140′ during a stapling motion, and then pass through the staple holes 151s′ of the cover 151′ to be released to the outside of the cartridge 150′.

In addition, a slit 151w′ may be formed in the cover 151′ in the lengthwise direction of the cover 151′. A blade of a main body 142′ of the working member 140′ may protrude out of the cartridge 150′ through the slit 151w′. As the blade of the main body 142′ of the working member 140′ passes along the slit 151w′, stapled tissue may be cut.

In an alternative embodiment, the cartridge 150′ may include a case 152′, and after the case 152′ is arranged in the accommodation part 101a′ of the first jaw 101′, the cartridge 150′ may be arranged in the case 152′.

A plurality of staples 153′ may be arranged inside the cartridge accommodation part 101a′ of the first jaw 101′. As the working member 140′ moves linearly in one direction, the plurality of staples 153′ may be sequentially pushed up from the inside of the cartridge accommodation part 101a′ of the first jaw 101′ to the outside, thereby performing suturing, that is, stapling. Here, the material of the staples 153′ may include a material that is durable and does not have an abnormal effect on the human body, and may include, for example, titanium or stainless steel.

In addition, the release member 1531′ may be further arranged between the cartridge accommodation part 101a′ of the first jaw 101′ and the staples 153′. In other words, it may also be described that the staple 153′ is arranged on the release member 1531′. In this case, the working member 140′ may linearly move in one direction to push up the release member 1531′, and the release member 1531′ may push up the staples 153′.

This may include a case in which the working member 140′ directly pushes up the staples 153′, and a case in which the working member 140′ pushes up the release member 1531′ such that the release member 1531′ pushes up the staples 153′ (i.e., a case in which the working member 140′ indirectly pushes up the staples 153′), and thus, it may be described that the working member 140′ pushes up the staples 153′.

As described above, the working member 140′ may be arranged inside the cartridge accommodation part 101a′ of the first jaw 101′. In addition, the working member 140′ may include the wedge WDG or may be used together with the wedge WDG, and when the working member 140′ moves, the wedge WDG moves together such that the wedge WDG directly pushes up the staple 153′, or the wedge WDG pushes up the release member 1531′ to push up the staple 153′.

As described above, as the forward wire 110′ moves, that is, as the forward wire 110′ is pulled, the working member 140′ connected thereto may move forward toward the distal end 101d′ of the first jaw 101′.

Through the forward movement of the working member 140′, the wedge WDG may push up the release member 1531′ to raise the staple 153′, and accordingly, cutting may be performed through the blade of the main body 142′ of the working member 140′. In addition, in an alternative embodiment, in an end tool in which a backward wire is connected to the working member 140′, the working member 140′ may be moved backward toward the proximal end 101p′ of the first jaw 101′ by pulling the backward wire.

Referring to FIGS. 12A to 13, in the state illustrated in FIG. 12A, as the working member 140′ moves in the direction of arrow A1 of FIG. 12B, the wedge WDG, specifically an inclined surface WDG1 of the wedge WDG pushes up the release member 1531′, and the release member 1531′ pushes up the lower side of the staple 153′. This causes the staple 153′ to be ejected from the first jaw 101′ and the cartridge 150′.

In this state, when the working member 140′ moves further in the direction of arrow A2 of FIG. 12C, the ejected staple 153′ is continuously pushed up by the working member 140′ while being in contact with the lower surface of the second jaw 102, for example, an anvil, both ends of the staple 153′ are bent, and thus, stapling is performed.

As the above motions are continuously performed, stapling may be sequentially performed from the staple 153′ on the side of the proximal end 101p′ (see FIG. 9) to the staple 153′ on the side of the distal end 101d′ (see FIG. 9) among the plurality of staples 153′, as illustrated in FIG. 13.

<Driving part>

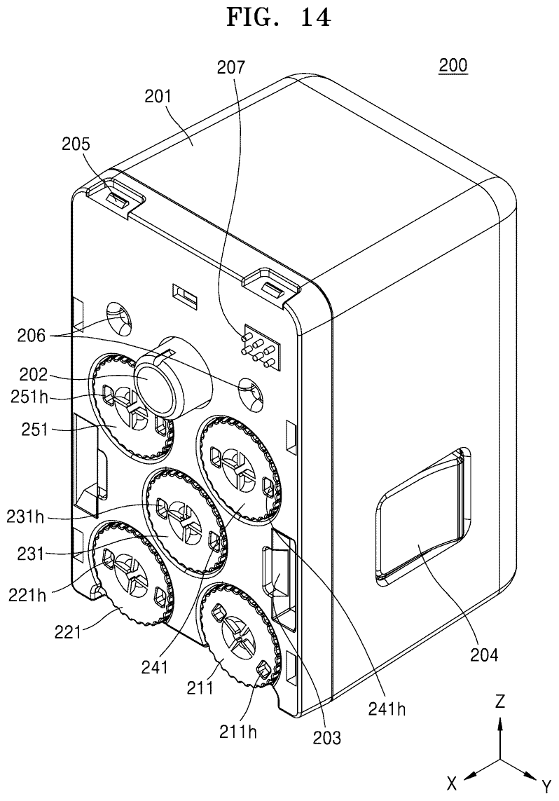

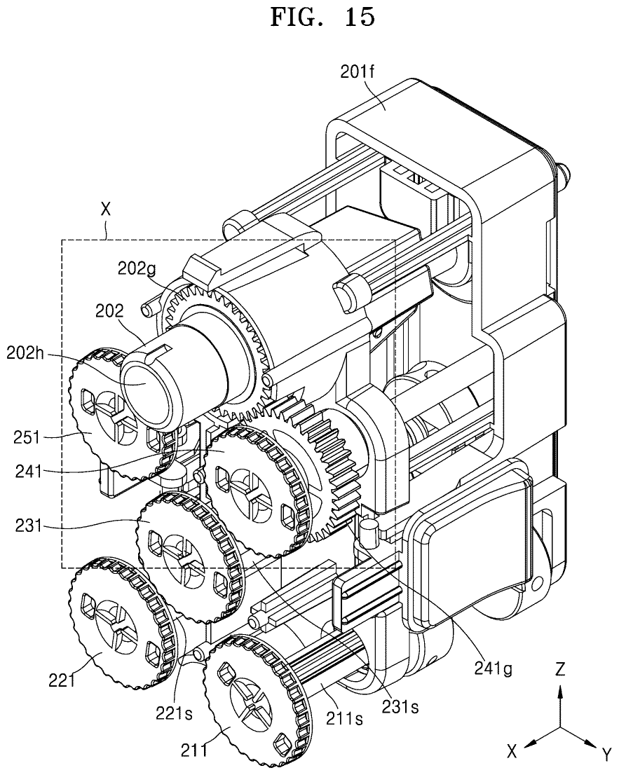

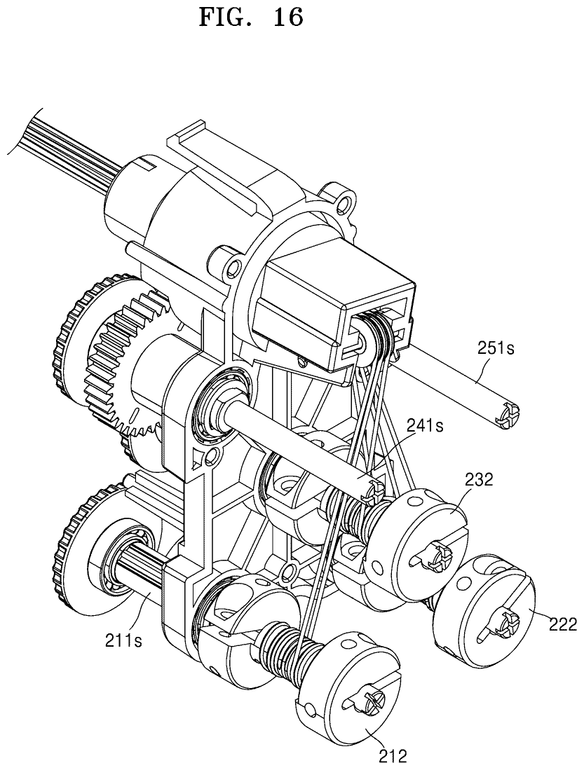

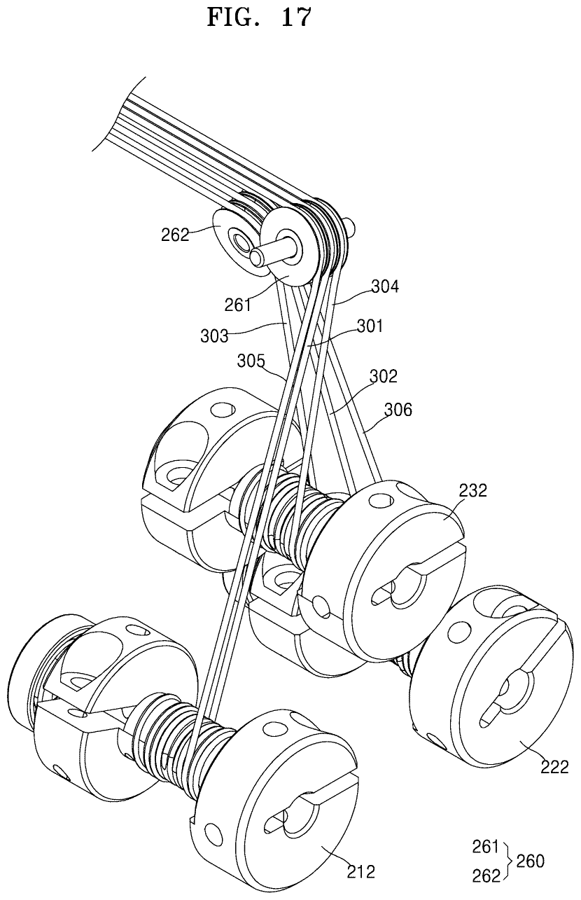

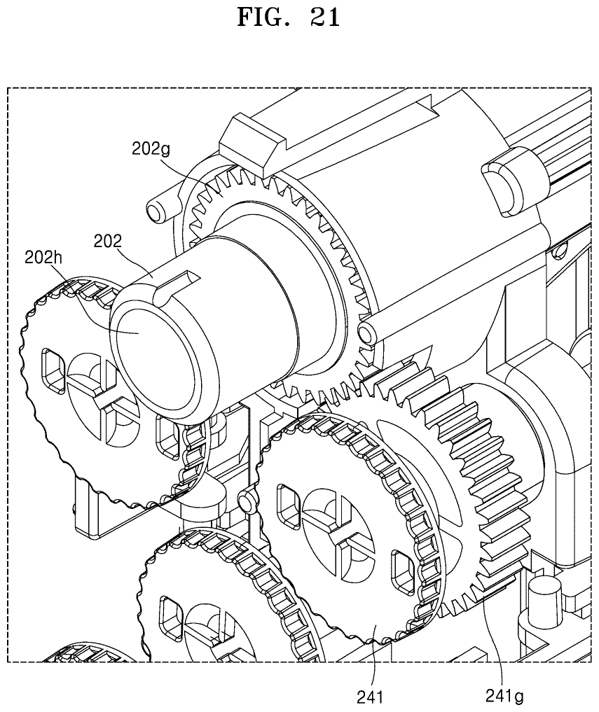

FIG. 14 is a perspective view of a driving part of the instrument module of FIG. 2, and FIGS. 15 and 16 are diagrams illustrating an internal structure of a driving part. FIGS. 17 to 20 are diagrams for describing an arrangement structure of pulleys and wires inside the driving part of FIG. 14. FIG. 21 is an enlarged view of portion X of FIG. 15.

Referring to FIGS. 14 to 21, the instrument module 30 according to an embodiment of the present disclosure may be mounted on the robotic arm units 21, 22, and 23 and the manipulation module 600 of the slave robot 20. The instrument module 30 may include the driving part 200.

The driving part 200 may receive a driving force from the outside and transmit the received driving force to the end tool 100. For example, the driving part 200 may receive a driving force from the robotic arm unit 21, 22, or 23 or the manipulation module 600 of the slave robot 20, and transmit the received driving force to the end tool 100 to enable various motions of the end tool. In other words, in a case in which the driving part 200 is mounted on the robotic arm unit 21, 22, or 23, the driving part 200 may serve as an interface connecting the robotic arm unit 21, 22, or 23 to the instrument module 30, or in a case in which the driving part 200 is mounted on the manipulation module 600, the driving part 200 may serve as an interface connecting the manipulation module 600 to the instrument module 30. Hereinafter, unless otherwise specified, the instrument module 30 will be described as including the end tool 100 described above with reference to FIGS. 5 to 7, but is not limited thereto, and may include the end tool 100′ described above with reference to FIGS. 8 to 11, or another end tool.

The driving part 200 may include a housing 201 and a frame 201f.

The housing 201 may form the overall exterior of the driving part 200. The housing 201 may include a case approximately in the shape of a box, and a cover arranged in front of the case.

The housing 201 may include a connection part fixing member 202. The connection part fixing member 202 is a member to which the connection part 400 of the instrument module 30 is connected. The connection part fixing member 202 may include a connection part accommodation hole 202h into which the connection part 400 is inserted.

The connection part fixing member 202 may be formed to extend such that at least one area thereof has a length. For example, the connection part fixing member 202 may be formed to extend to have a length in a direction parallel to the lengthwise direction of the connection part 400. Here, the connection part accommodation hole 202h may be formed in the portion of the connection part fixing member 202 extending to have the length, so as to correspond to the lengthwise direction.

The connection part 400 may be inserted and fixed into the connection part accommodation hole 202h. For example, the connection part 400 may be inserted into the connection part accommodation hole 202h such that at least one area of the connection part 400 overlaps the connection part accommodation hole 202h.

The connection part 400 may be inserted and fastened into the connection part accommodation hole 202h. The connection part 400 is fastened to the connection part accommodation hole 202h, and thus, when the connection part fixing member 202 rotates, the connection part 400 may also rotate. This is associated with a roll motion of the end tool 100 as will be described below.

The housing 201 may include a shape and structure that allow the instrument module 30 to be mounted on the robotic arm unit 21, 22, or 23 or the manipulation module 600.

For example, a fastening groove 203 may be formed in the housing 201. As described below, the fastening groove 203 is a portion in which a first fastening part 503 formed in the robotic arm unit 21, 22, or 23, or a second fastening part 622 formed in the manipulation module 600 is inserted. By inserting and fixing the first fastening part 503 into the fastening groove 203, the instrument module 30 may be mounted and fixed on the robotic arm unit 21, 22, or 23 not to be separated. In addition, by inserting and fixing the second fastening part 622 into the fastening groove 203, the instrument module 30 may be mounted and fixed on the manipulation module 600 not to be separated.

The driving part 200 may further include a detachment lever 204. For example, the detachment lever 204 may be provided in the housing 201, and specifically, may be provided in the case or the cover.

The detachment lever 204 is a member that releases the fixation of the first fastening part 503 or the second fastening part 622 (hereinafter, collectively referred to as a fastening part) inserted into the fastening groove 203. For example, a ring may be formed on the first fastening part 503 and the second fastening part 622, and a protrusion or a hook that may be caught on the ring may be formed on the detachment lever 204. When the first fastening part 503 or the second fastening part 622 is inserted into the fastening groove 203, the ring may be caught on the protrusion or hook such that the instrument module 30 may not be separated from to the robotic arm unit 21, 22, or 23 or the manipulation module 600. Accordingly, when the user manipulates the detachment lever 204, the hook caught on the protrusion or hook may be released to be separated from the protrusion or hook, and the instrument module 30 may be separated from the robotic arm unit 21, 22, or 23 or the manipulation module 600.

In an embodiment, the detachment lever 204 may be formed to be rotatable around a position inside the housing 201. For example, the detachment lever 204 may be formed to be rotatable to reciprocate between the inside and the outside of the housing 201. In detail, when the user presses the detachment lever 204 in one direction, the detachment lever 204 may rotate, and the rotation of the detachment lever 204 may cause the protrusion or hook to be separated from the fastening part, such that the coupling between the instrument module 30, and the robotic arm unit 21, 22, or 23 or the instrument module 30, and the manipulation module 600 is released.

However, the detachment lever 204 is not limited thereto, and may be configured in a sliding manner or the like, and in addition, may be configured in various manners as long as the instrument module 30 may be mounted on and separated from the robotic arm unit 21, 22, or 23 or the manipulation module 600.

For example, the housing 201 may further include a coupling jaw 205. The coupling jaw 205 may be formed in a shape protruding outward from the housing 201. The coupling jaw 205 may perform a function of firmly fixing the instrument module 30 when it is mounted on the robotic arm unit 21, 22, or 23 or the manipulation module 600. For example, a coupling ring 505 to be locked to the coupling jaw 205 may be formed on the robotic arm units 21, 22, and 23 and the manipulation module 600. Thus, once the instrument module 30 is mounted on the robotic arm unit 21, 22, or 23 or the manipulation module 600, the instrument module 30 may remain mounted on the robotic arm unit 21, 22, or 23 or the manipulation module 600 unless the instrument module 30 is forcibly separated by manipulation by the user.

For example, an insertion groove 206 may be formed on the housing 201. The insertion groove 206 is a portion into which an insertion protrusion 506 formed on the robotic arm unit 21, 22, or 23 or the manipulation module 600 is inserted. The insertion protrusion 506 is a portion formed on the robotic arm unit 21, 22, or 23 or the manipulation module 600, and may be formed to extend outward to have a certain length. In this case, because the instrument module 30 is mounted on the robotic arm unit 21, 22, or 23 or the manipulation module 600 such that the insertion protrusion 506 is inserted into the insertion groove 206, the coupling between the instrument module 30, and the robotic arm unit 21, 22, or 23, or between the instrument module 30 and the manipulation module 600 may maintain consistency. In other words, the instrument module 30 may be mounted at a position where the insertion protrusion 506 is inserted into the insertion groove 206, that is, at a predetermined position of the robotic arm unit 21, 22, or 23 or the manipulation module 600.

As such, the housing 201 may include various shapes and structures that allow the instrument module 30 to be mounted on the robotic arm unit 21, 22, or 23 or the manipulation module 600, and accordingly, the instrument module 30 may be stably coupled to the robotic arm unit 21, 22, or 23 or the manipulation module 600 and may not be unexpectedly separated during surgery after the coupling. In addition, while mounted on the robotic arm unit 21, 22, or 23 or the manipulation module 600, the instrument module 30 may not unintentionally rotate independently or may not be partially separated, and thus, the safety of surgery may be secured. In addition, by adopting the mechanical coupling structure described above, when a problem occurs with the surgical robot while performing surgery by using the robotic arm unit 21, 22, or 23, or when surgery is required in a hand-held manner, the user may intuitively and easily remove the instrument module 30 from the robotic arm unit 21, 22, or 23 and then reinstall it on the manipulation module 600 to continuously perform the surgery.

However, the present disclosure is not limited thereto, and in addition to the above mounting structure, the instrument module 30 may be modified and used in a configuration that allows the instrument module 30 to be easily and stably mounted and coupled to the robotic arm unit 21, 22, or 23 or the manipulation module 600.

For example, the driving part 200 may include an instrument communication terminal 207. The instrument communication terminal 207 is a portion to be connected to a robot communication terminal 507 provided in a surgical robot. When the instrument module 30 is mounted on the robotic arm unit 21, 22, or 23, the instrument communication terminal 207 may be electrically connected to the robot communication terminal 507.

The instrument communication terminal 207 and the robot communication terminal 507 are portions that transmit and receive signals regarding the condition or the like of the instrument module 30. For example, the surgical robot may receive, through the instrument communication terminal 207 and the robot communication terminal 507, information such as whether the instrument module 30 is used, a usage history, the date of manufacture, and the expiration date, and the like of the instrument module 30. That is, it may be understood that a driving force for operating the instrument module 30 is transmitted from the surgical robot in a mechanical manner as will be described below, but information about the instrument module 30 is electrically transmitted through the instrument communication terminal 207 and the robot communication terminal 507.

The driving part 200 according to an embodiment of the present disclosure may include at least one driving member that receives a driving force for operating the end tool 100 from the outside.

Here, the outside may refer to the slave robot 20 or the robotic arm unit 21, 22, or 23 when the instrument module 30 is mounted on the robotic arm unit 21, 22, and 23, and may refer to the manipulation module 600 when the instrument module 30 is mounted on the manipulation module 600. In more detail, it may also be described that the driving member is a component corresponding to an interface that mechanically/physically connects the driving force transmitted between the instrument module 30 and the robotic arm unit 21, 22, or 23, and between the instrument module 30 and the manipulation module 600.

The driving member may include a pitch driving member 231 that receives a driving force for controlling a pitch motion of the end tool 100 from the outside, and a jaw driving member that receives a driving force for controlling a rotational motion of the jaw 103 from the outside.

In a specific embodiment, the driving part 200 may include a first jaw driving member 211 corresponding to the first jaw 101 of the end tool 100, and a second jaw driving member 221 corresponding to the second jaw 102.

The driving part 200 may include a shaft extending in one direction from the jaw driving member. For example, the shaft may be formed with the jaw driving member as one body, or may be formed as a separate member and then coupled to the jaw driving member. Here, the shaft may function as a rotation shaft of the jaw driving member.

In detail, the driving part 200 may include a first rotation shaft 211s extending in one direction from the first jaw driving member 211, and a second rotation shaft 221s extending in one direction from a second jaw power transmission plate 521.

As the first jaw driving member 211 rotates, the first rotation shaft 211s may rotate together. As the second jaw driving member 221 rotates, the second rotation shaft 221s may rotate together.

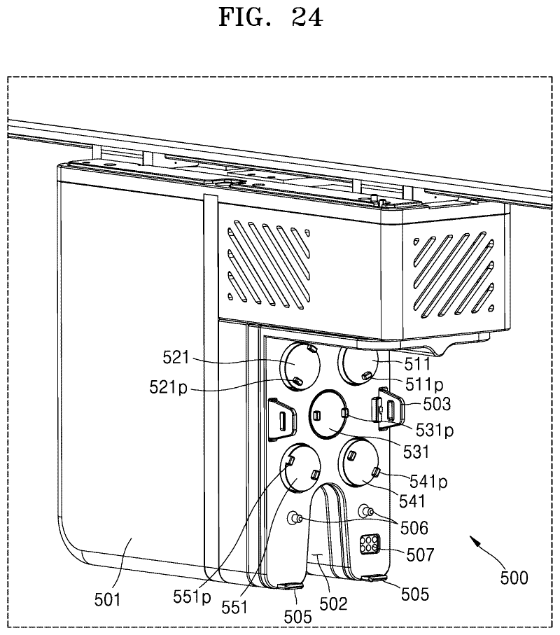

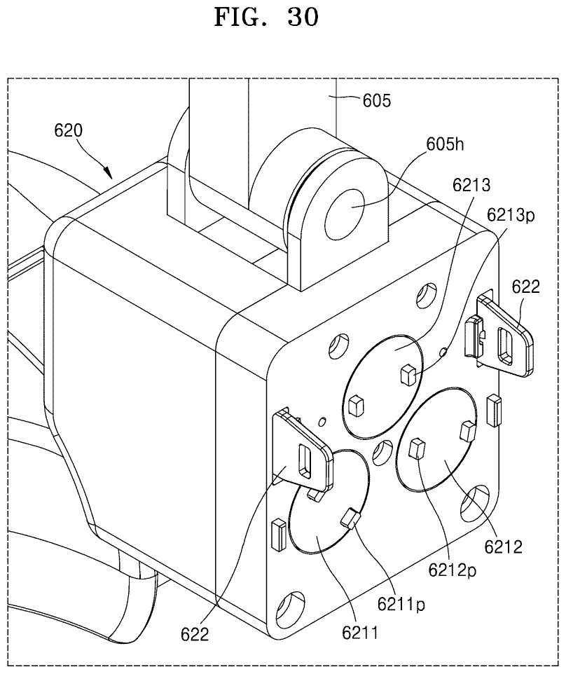

At least one first insertion groove 211h may be formed on the first jaw driving member 211. The first insertion groove 211h is a portion into which a first protrusion 511p (see FIG. 24) formed on a first jaw power transmission plate 511 of a power transmission unit 500, or a 1-1st protrusion 6211p (see FIG. 30) formed on a first jaw power transmission member 6211 of the manipulation module 600 is inserted.

A plurality of first insertion grooves 211h may be formed, and it is preferable that the number of first insertion grooves 211h corresponds to the number of first protrusions 511p (see FIG. 24) and/or the number of 1-1st protrusions 6211p (see FIG. 30). As such, when a plurality of first protrusions 51 lp (see FIG. 24) and/or a plurality of 1-1st protrusions 621 1p (see FIG. 30) are respectively inserted into the first insertion grooves 211h, a rotational force of the first jaw power transmission plate 511 provided in the power transmission unit 500 of the robotic arm unit 21, 22, or 23, and/or the first jaw power transmission member 6211 of the manipulation module 600 may be efficiently transmitted to the first jaw driving member 211.

At least one second insertion groove 221h may be formed on the second jaw driving member 221. The second insertion groove 221h is a portion into which a second protrusion 521p (see FIG. 24) formed on the second jaw power transmission plate 521 of the power transmission unit 500, or a 2-1st protrusion 6212p (see FIG. 30) formed on a second jaw power transmission member 6212 of the manipulation module 600 is inserted.

A plurality of second insertion grooves 221h may be formed, and it is preferable that the number of second insertion grooves 221h corresponds to the number of second protrusions 521p (see FIG. 24) and/or the number of 2-1st protrusions 6212p (see FIG. 30). As such, when a plurality of second protrusions 521p (see FIG. 24) and/or a plurality of 2-1st protrusions 6212p (see FIG. 30) are respectively inserted into the second insertion grooves 221h, a rotational force of the second jaw power transmission plate 521 provided in the power transmission unit 500 of the robotic arm unit 21, 22, or 23, and/or the second jaw power transmission member 6212 of the manipulation module 600 may be efficiently transmitted to the second jaw driving member 221.

The driving part 200 may be connected to the jaw driving member and may include an instrument jaw pulley that rotates together with the jaw driving member.

In detail, the driving part 200 may be connected to the first jaw driving member 211 by the first rotation shaft 211s, and may include a first jaw pulley 212 that rotates together with the first jaw driving member 211 and the first rotation shaft 211s. In addition, the driving part 200 may be connected to the second jaw driving member 221 by the second rotation shaft 221s, and may include a second jaw pulley 222 that rotates together with the second jaw driving member 221 and the second rotation shaft 221s. In other words, it may also be described that, when the first jaw driving member 211 is rotated by a driving force received from the outside, the instrument first jaw pulley 212 rotates together with the first jaw driving member 211. Similarly, it may also be described that, when the second jaw driving member 221 is rotated by a driving force received from the outside, the instrument second jaw pulley 222 rotates together with the second jaw driving member 221.

A jaw wire may be wound around the instrument jaw pulley, and the instrument jaw pulley may wind or unwind the jaw wire by rotation.

In detail, the wires 301 and 305 may be wound around the instrument first jaw pulley 212, and the instrument first jaw pulley 212 may wind or unwind the wires 301 and 305. Here, the wires 301 and 305 are wires arranged to extend from the end tool 100 to the inside of the driving part 200 through the connection part 400. Here, the wire 301 and the wire 305 may be referred to as first jaw wires.

Thus, when the first jaw driving member 211 is rotated by a driving force transmitted from the outside, the instrument first jaw pulley 212 may rotate together, and through this rotation, the wires 301 and 305 may be wound or unwound to rotate the first jaw 101 pulley 111 connected to the wires 301 and 305 such that the first jaw 101 is rotated.

Similarly, the wires 302 and 306 may be wound around the instrument second jaw pulley 222, and the instrument second jaw pulley 222 may wind or unwind the wires 302 and 306. Here, the wires 302 and 306 are wires arranged to extend from the end tool 100 to the inside of the driving part 200 through the connection part 400. Here, the wire 302 and the wire 306 may be referred to as second jaw wires.

Thus, when the second jaw driving member 221 is rotated by a driving force transmitted from the outside, the instrument second jaw pulley 222 may rotate together, and through this rotation, the wires 302 and 306 may be wound or unwound to rotate the second jaw 102 pulley 121 connected to the wires 302 and 306 such that the second jaw 102 is rotated.

The driving part 200 may include a shaft extending in one direction from the pitch driving member 231, that is, a third rotation shaft 231s. For example, the third rotation shaft 231s may be formed with the pitch driving member 231 as one body, or may be formed as a separate member and then coupled to the pitch driving member 231. Here, the third rotation shaft 231s may function as a rotation shaft of the pitch driving member 231.

As the pitch driving member 231 rotates, the third rotation shaft 231s may rotate together.

At least one third insertion groove 231h may be formed on the pitch driving member 231. The third insertion groove 231h is a portion into which a third protrusion 531p (see FIG. 24) formed on the pitch power transmission plate 531 of the power transmission unit 500, or a 3-1st protrusion 6213p (see FIG. 30) formed on a pitch power transmission member 6213 of the manipulation module 600 is inserted.

A plurality of third insertion grooves 231h may be formed, and it is preferable that the number of third insertion grooves 231h corresponds to the number of third protrusions 531p (see FIG. 24) and/or the number of 3-1st protrusions 6213p (see FIG. 30). As such, when a plurality of third protrusions 531p (see FIG. 24) and/or a plurality of 3-1st protrusions 6213p (see FIG. 30) are respectively inserted into the third insertion grooves 231h, a rotational force of the pitch power transmission plate 531 provided in the power transmission unit 500 of the robotic arm unit 21, 22, or 23, and/or the pitch power transmission member 6213 of the manipulation module 600 may be efficiently transmitted to the pitch driving member 231.

The driving part 200 may be connected to the pitch driving member 231 and may include an instrument pitch pulley 232 that rotates together with the pitch driving member 231. In other words, it may also be described that, when the pitch driving member 231 is rotated by a driving force received from the outside, the instrument pitch pulley 232 rotates together with the pitch driving member 231.

A pitch wire may be wound around the instrument pitch pulley 232, and the instrument pitch pulley 232 may wind or unwind the pitch wire by rotation.

In detail, the wires 303 and 304 may be wound around the instrument pitch pulley 232, and the instrument pitch pulley 232 may wind or unwind the wires 303 and 304. Here, the wires 303 and 304 are wires arranged to extend from the end tool 100 to the inside of the driving part 200 through the connection part 400.

Thus, when the pitch driving member 231 is rotated by a driving force transmitted from the outside, the instrument pitch pulley 232 may rotate together, and through this rotation, the wires 303 and 304 may be wound or unwound to rotate the end tool 100 pitch pulley 131 connected to the wires 303 and 304 such that the end tool 100 is rotated.

For example, the driving part 200 may further include an instrument roll power transmission plate 241. In a case in which the instrument module 30 is mounted on the robotic arm unit 21, 22, or 23, the instrument roll power transmission plate 241 may receive a driving force for causing the end tool 100 or the connection part 400 to perform a roll motion, from a roll power transmission plate 541 provided in the power transmission unit 500 of the robotic arm unit 21, 22, or 23.

In an alternative embodiment, the instrument module 30 includes the instrument roll power transmission plate 241, but the instrument roll power transmission plate 241 may be operated only in a case in which the instrument module 30 is mounted on the robotic arm unit 21, 22, or 23. In more detail, in a case in which the instrument module 30 is mounted on the robotic arm unit 21, 22, or 23, roll rotation of the end tool 100 or the connection part 400 may be performed by a motor (see FIG. 25) provided in the robotic arm unit 21, 22, or 23. In other words, a driving force generated from the motor (see FIG. 25) provided in the robotic arm unit 21, 22, or 23 is transmitted to the instrument module 30 through the instrument roll power transmission plate 241, and the transmitted driving force causes the end tool 100 and/or the connection part 400 to perform roll rotation. Meanwhile, in a case in which the instrument module 30 is mounted on the manipulation module 600, the instrument roll power transmission plate 241 is not operated. This is because, in a case in which the instrument module 30 is mounted on the manipulation module 600, a roll motion of the end tool 100 and/or the connection part 400 may be implemented by the user holding the manipulation module 600 and directly roll-rotating the manipulation module 600. For example, the user may cause the end tool 100 and/or the connection part 400 to perform roll rotation, by mounting the instrument module 30 on the manipulation module 600 and then holding and roll-rotating the manipulation module 600. However, even when the instrument module 30 is directly roll-rotated by the manipulation module 600, the instrument module 30 according to the present disclosure has compatibility to be mounted and operated on a surgical robot or on the manipulation module 600, and thus, the driving part 200 includes a component for roll rotation of the end tool 100 and/or the connection part 400, for example, the instrument roll power transmission plate 241.

Here, a configuration for implementing a roll rotational motion of the instrument module 30 will be described in more detail.

In an embodiment, referring to FIG. 21, the driving part 200 may further include a first roll gear 241g that is connected to the instrument roll power transmission plate 241 and rotates together. For example, a fourth rotation shaft 241s may be connected to the instrument roll power transmission plate 241, and the first roll gear 241g may be connected to the fourth rotation shaft 241s. Here, the driving part 200 may further include a second roll gear 202g to be engaged with the first roll gear 241g and thus rotate together with rotation of the first roll gear 241g. Here, the second roll gear 202g may be provided to be connected to the connection part fixing member 202. For example, the second roll gear 202g may be formed with the connection part fixing member 202 as one body, or may be provided as a separate member from the fixing member and then coupled to the connection part fixing member 202.

At least one fourth insertion groove 241h may be formed on the instrument roll power transmission plate 241. The fourth insertion groove 241h is a portion into which a fourth protrusion 541p (see FIG. 24) formed on the roll power transmission plate 541 is inserted.

A plurality of fourth insertion grooves 241h may be formed, and it is preferable that the number of fourth insertion grooves 241h corresponds to the number of fourth protrusions 541p. As such, when a plurality of fourth protrusions 541p are respectively inserted into the fourth insertion grooves 241h, a rotational force of the roll power transmission plate 541 provided in the power transmission unit 500 of the robotic arm unit 21, 22, or 23 may be efficiently transmitted to the instrument roll power transmission plate 241.

To describe in detail the implementation principle of a roll rotational motion of the instrument module 30, the motor (see FIG. 25) provided on the robotic arm unit 21, 22, or 23 is driven after the instrument module 30 is mounted on the robotic arm unit 21, 22, or 23, a driving force of the motor may be transmitted to the instrument roll power transmission plate 241. When the instrument roll power transmission plate 241 is rotated by the driving force, the first roll gear 241g connected to the instrument roll power transmission plate 241 may rotate together. As the first roll gear 241g rotates, the second roll gear 202g may also rotate together. As the second roll gear 202g rotates, the connection part fixing member 202 connected to the second roll gear 202g may also rotate together, accordingly, the connection part 400 inserted into the connection part fixing member 202 may also rotate, and thus, the end tool 100 may roll-rotate. To this end, it is preferable that the connection part 400 is fixed after being inserted into the connection part fixing member 202, in other words, it is preferable that the connection part 400 is inserted into the connection part fixing member 202 and then fixed such that the connection part 400 does not rotate with respect to the connection part fixing member 202.

Meanwhile, because the instrument roll power transmission plate 241 and the first roll gear 241g need to receive a driving force for a roll motion of the instrument module 30 from the robotic arm unit 21, 22, or 23, and then directly transmit the driving force to the second roll gear 202g and the connection part fixing member 202, it is preferable that the instrument roll power transmission plate 241 is arranged closer to the connection part fixing member 202 than other driving members. Thus, the instrument roll power transmission plate 241 may be arranged closer to the connection part fixing member 202 than other driving members, especially than the jaw driving member and the pitch driving member 231.

In an embodiment, the driving member may include an instrument translational power transmission plate 251 that receives a driving force for controlling a motion of the working member 140′ of the end tool 100′ from the outside.

The driving part 200 may include a shaft extending in one direction from the instrument translational power transmission plate 251, that is, a fifth rotation shaft 251s. For example, the fifth rotation shaft 251s may be formed with the instrument translational power transmission plate 251 as one body, or may be formed as a separate member and then coupled to the instrument translational power transmission plate 251. Here, the fifth rotation shaft 251s may function as a rotation shaft of the instrument translational power transmission plate 251.

As the instrument translational power transmission plate 251 rotates, the fifth rotation shaft 251s may rotate together.

At least one fifth insertion groove 251h may be formed on the instrument translational power transmission plate 251. The fifth insertion groove 251h is a portion into which a fifth protrusion 551p (see FIG. 24) formed on a translational power transmission plate 551 of the power transmission unit 500 is inserted.

A plurality of fifth insertion grooves 251h may be formed, and it is preferable that the number of fifth insertion grooves 251h corresponds to the number of fifth protrusions 551p (see FIG. 24). As such, when a plurality of fifth protrusions 551p are respectively inserted into the fifth insertion grooves 251h, a rotational force of the translational power transmission plate 551 provided in the power transmission unit 500 of the robotic arm unit 21, 22, or 23 may be efficiently transmitted to the instrument translational power transmission plate 251.

In an alternative embodiment, although not illustrated in the drawings, the driving part 200 may further include an instrument translational pulley that is connected to the instrument translational power transmission plate 251 and rotates together with the instrument translational power transmission plate 251. In other words, it may also be described that, when the instrument translational power transmission plate 251 is rotated by a driving force received from the outside, the instrument translational pulley rotates together.

In this case, the forward wire (see FIG. 10) may be wound around the instrument translational pulley, and the instrument translational pulley may wind or unwind the forward wire by rotation.

However, the configuration for controlling a translational motion of the working member 140′ of the end tool 100′ according to the present disclosure may be variously modified, and thus, detailed descriptions will be omitted.

Meanwhile, as will be described below, a configuration for transmitting a driving force for controlling a translational motion of the working member 140′ to the instrument module 30 is not illustrated in the manipulation module 600 according to an embodiment of the present disclosure, but may be appropriately adopted with reference to the configuration for transmitting the driving force for controlling the pitch motion of the end tool 100′, or the rotational motion of the jaw 103 to the instrument module 30. In this case, the instrument translational power transmission plate 251 may be appropriately connected to the above configuration of the manipulation module 600 and appropriately transmit a driving force for controlling a translational motion of the working member 140′ received from the manipulation module 600, to the working member 140′ of the end tool 100′.

Hereinafter, an arrangement structure of various pulleys and wires provided in the driving part 200 will be described in detail.

In an embodiment, referring to FIGS. 15 to 20, an instrument jaw pulley and the instrument pitch pulley 232 may be arranged inside the driving part 200. In a specific embodiment, the instrument jaw pulley may include an instrument first jaw pulley 212 and an instrument second jaw pulley 222.

The instrument first jaw pulley 212 may be connected to the first rotation shaft 211s, and the first rotation shaft 211s may be fixed to the housing 201 or the frame 201f. For example, the first rotation shaft 211s may be fixed to the case or the cover of the housing 201, or may be fixed to any area of the frame 201f. The instrument second jaw pulley 222 may be connected to the second rotation shaft 221s, and the second rotation shaft 221s may be fixed to the housing 201 or the frame 201f. For example, the second rotation shaft 221s may be fixed to the case or the cover of the housing 201, or may be fixed to any area of the frame 201f. The instrument pitch pulley 232 may be connected to the third rotation shaft 231s, and the third rotation shaft 231s may be fixed to the housing 201 or the frame 201f. For example, the third rotation shaft 231s may be fixed to the case or the cover of the housing 201, or may be fixed to any area of the frame 201f. In other words, it may also be described that the pulleys 212, 222, and 232 may be connected to the rotation shafts 211s, 221s, and 231s, respectively, and the arrangement of the pulleys 212, 222, and 232 is determined by the relative positions of the rotation shafts 211s, 221s, and 231s, respectively.

The instrument jaw pulley may be arranged at a different height from the instrument pitch pulley 232 with respect to FIG. 15. For example, it may be described that the instrument jaw pulley is arranged at a different height from the instrument pitch pulley 232 with respect to the Z-axis direction of FIG. 15. In a specific embodiment, the instrument jaw pulley may be arranged lower than the instrument pitch pulley 232.

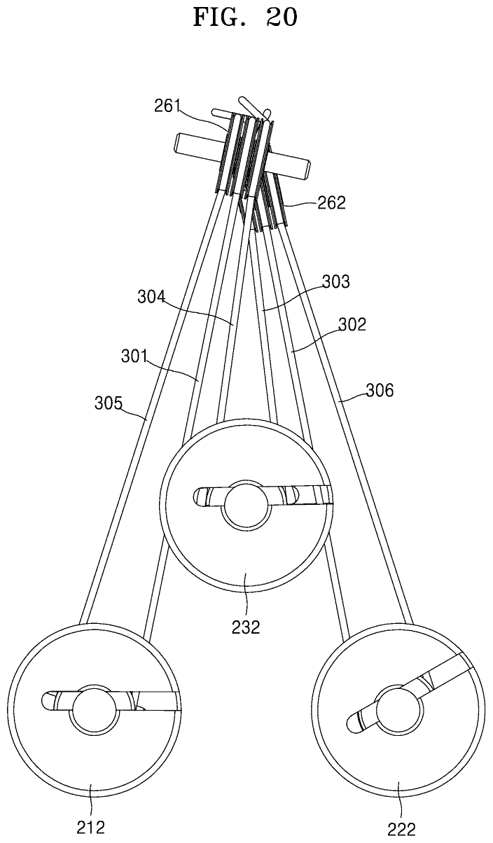

The instrument first jaw pulley 212 and the instrument second jaw pulley 222 may be arranged to be symmetrical to each other in the horizontal direction with respect to the instrument pitch pulley 232. For example, the instrument first jaw pulley 212 and the instrument second jaw pulley 222 may be arranged on the left and right with respect to FIG. 20. Here, that the instrument first jaw pulley 212 and the instrument second jaw pulley 222 are arranged on the left and right does not mean that they are arranged at exactly the same distance and angle with respect to the instrument pitch pulley 232, but may mean that they are arranged at left and right portions of the lower side with respect to the instrument pitch pulley 232.

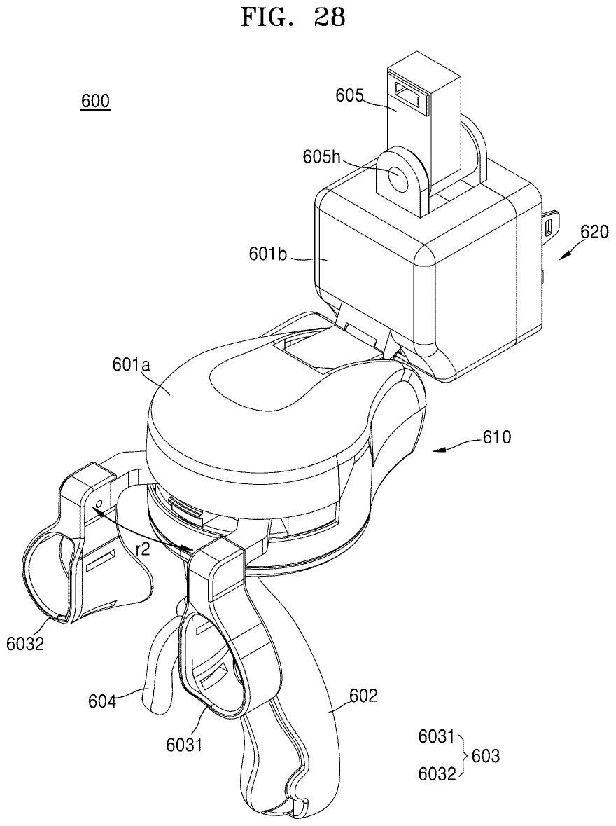

Due to the above arrangement, the pulleys 212, 222, and 232 may be efficiently arranged in a space, and the driving part 200 may be miniaturized. Furthermore, due to the arrangement, intuitive operation of the instrument module 30 according to the present disclosure may be implemented. For example, the user may operate the first jaw 101 and the second jaw 102 by manipulating an actuation manipulation part 603 (see FIG. 28) arranged on the left and right sides of the manipulation module 600, and this manipulation may enable rotation of the first jaw 101 and the second jaw 102 by rotating the instrument first jaw pulley 212 and the instrument second jaw pulley 222, which are arranged to be symmetrical to each other.

In particular, in the present disclosure, the symmetrical structure of the instrument first jaw pulley 212 and the instrument second jaw pulley 222 may be more important for intuitive operation of the instrument module 30. For example, it is preferable that a force applied to a first actuation manipulation part 6031 (see FIG. 28) of the manipulation module 600 for a motion of the first jaw 101, and a force applied to a second actuation manipulation part 6032 (see FIG. 28) of the manipulation module 600 for a motion of the second jaw 102 are similar to each other. In addition, it is preferable that, when the first actuation manipulation part 6031 (see FIG. 28) and the second actuation manipulation part 6032 (see FIG. 28) are rotated to similar degrees, the first jaw 101 and the second jaw 102 rotate similarly.

To this end, the first jaw 101 wire and the second jaw 102 wire may extend to the driving part 200 through the connection part 400, and then extend to the instrument first jaw pulley 212 and the instrument second jaw pulley 222 to be approximately symmetrical to each other.

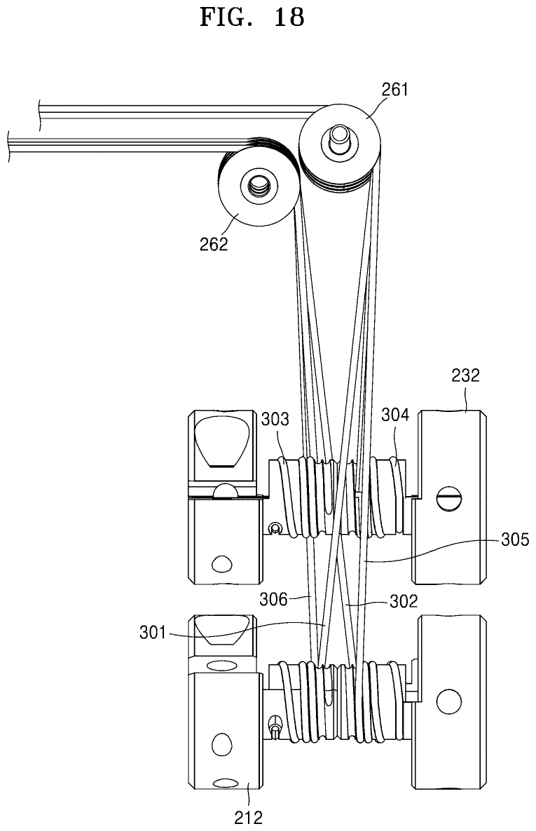

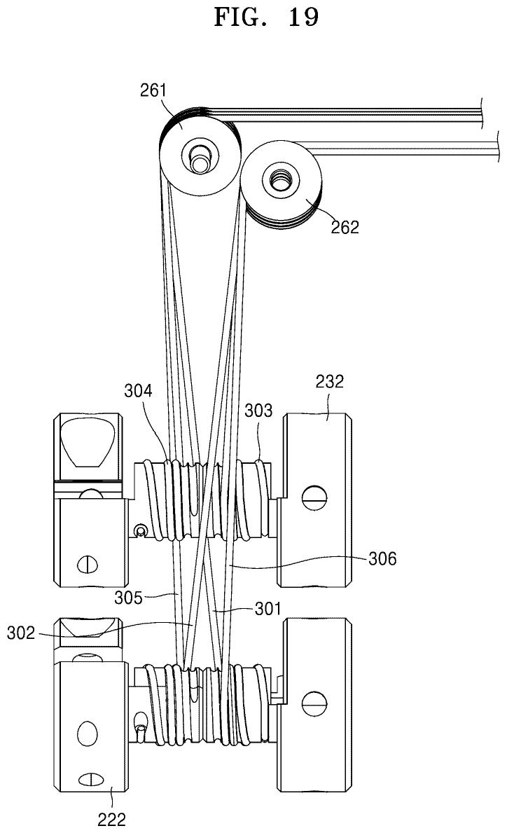

For example, the driving part 200 may further include at least one auxiliary pulley. The auxiliary pulley may change the paths of the wires 301, 302, 303, 304, 305, and 306 extending to the driving part 200. In detail, the auxiliary pulley may change the paths of the wires 301, 302, 303, 304, 305, and 306 to a direction that is not parallel to the connection part 400, and for example, may guide the wires 301, 302, 303, 304, 305, and 306 downward as illustrated in FIGS. 18 and 19.

The auxiliary pulley may include a first auxiliary pulley 261 and a second auxiliary pulley 262. The first auxiliary pulley 261 and the second auxiliary pulley 262 may each include a plurality of pulleys. For example, the first auxiliary pulley 261 and the second auxiliary pulley 262 may each include a number of pulleys corresponding to the number of wires wound therearound.

The wire 305, the wire 301, and the wire 304 may be sequentially wound around the first auxiliary pulley 261 from the outer side to the inner side. That is, the jaw wires 305 and 301 may be wound around the first auxiliary pulley 261 from the outer side to the inner first, and then the wire 301, which is one of the pitch wires, may be wound around the inner side. In this case, the first auxiliary pulley 261 may include at least three pulleys such that the wire 305, wire 301, and wire 304 are wound therearound.

The wire 306, the wire 302, and the wire 303 may be sequentially wound around the second auxiliary pulley 262 from the outer side to the inner side. That is, the jaw wires 306 and 302 may be wound around the second auxiliary pulley 262 from the outer side to the inner first, and then the wire 303, which is one of the pitch wires, may be wound around the inner side. In this case, the second auxiliary pulley 262 may include at least three pulleys such that the wire 306, wire 302, and wire 303 are wound therearound.

The first auxiliary pulley 261 and the second auxiliary pulley 262 may be arranged to be inclined in different directions. The first auxiliary pulley 261 may be inclined toward the instrument first jaw pulley 212, and the second auxiliary pulley 262 may be inclined toward the instrument second jaw pulley 222. In other words, the first auxiliary pulley 261 may be inclined in a direction to guide the wire 305 and the wire 301 to extend toward the instrument first jaw pulley 212, and the second auxiliary pulley 262 may be inclined in a direction to guide the wire 306 and the wire 302 to extend toward the instrument second jaw pulley 222. Thus, the wire 305 and the wire 301 may approach the instrument first jaw pulley 212 almost perpendicularly, and the wire 306 and the wire 302 may approach the instrument second jaw pulley 222 almost perpendicularly.

The wire 304 may be unwound from the first auxiliary pulley 261 to extend to one side of the instrument pitch pulley 232, and the wire 303 may be unwound from the second auxiliary pulley 262 to extend to the other side of the instrument pitch pulley 232. At this time, the wire 304 and the wire 303 may approach opposite sides of the instrument pitch pulley 232 and then be wound around the instrument pitch pulley 232 in opposite directions. In other words, in FIG. 20, the wire 304 may approach the left side of the instrument pitch pulley 232 to be wound counterclockwise around the instrument pitch pulley 232, and the wire 303 may approach the right side of the instrument pitch pulley 232 to be wound clockwise around the instrument pitch pulley 232.

Meanwhile, in the present embodiment, it has been described that a pair of auxiliary pulleys each including three pulleys (a total of six pulleys) are provided, but more auxiliary pulleys may be provided as necessary, and three pairs of auxiliary pulleys each including two pulleys may be provided. For example, there may be provided one set of auxiliary pulleys around which the first jaw wires 301 and 305 are wound, one set of auxiliary pulleys around which the second jaw wires 302 and 306 are wound, and one set of auxiliary pulleys around which the pitch wires 303 and 304 are wound. In this case, each auxiliary pulley may be arranged to be appropriately inclined toward the instrument jaw pulley and the instrument pitch pulley.

As such, due to the arrangement of the wires 301, 302, 303, 304, 305, and 306, and the pulleys 212, 222, and 232, the angles at which the wires 301, 302, 303, 304, 305, and 306 enter the pulleys 212, 222, and 232 are almost perpendicular, thus, the friction loss and force loss of the wires 301, 302, 303, 304, 305, and 306 may be reduced, and furthermore, wear may be prevented. In addition, the arrangement of the wires 301, 302, 303, 304, 305, and 306 and the pulleys 212, 222, and 232 enables intuitive operation of the end tool 100 by the user more intuitively and efficiently.

In addition, although not illustrated in the drawings, in a case in which the instrument module 30 includes an end tool (e.g., 100′ of FIG. 8) for performing a motion by the working member 140′, such as stapling, an additional pulley for controlling a translational motion of the working member 140′ may be added. In this case, the pulley for controlling the translational motion of the working member 140′ may be arranged to be connected to the fifth rotation shaft 251s, and the forward wire 110′ may be wound around the pulley for controlling the translational motion of the working member 140′.

### Embodiment in which Instrument Module is Mounted on Slave Robot

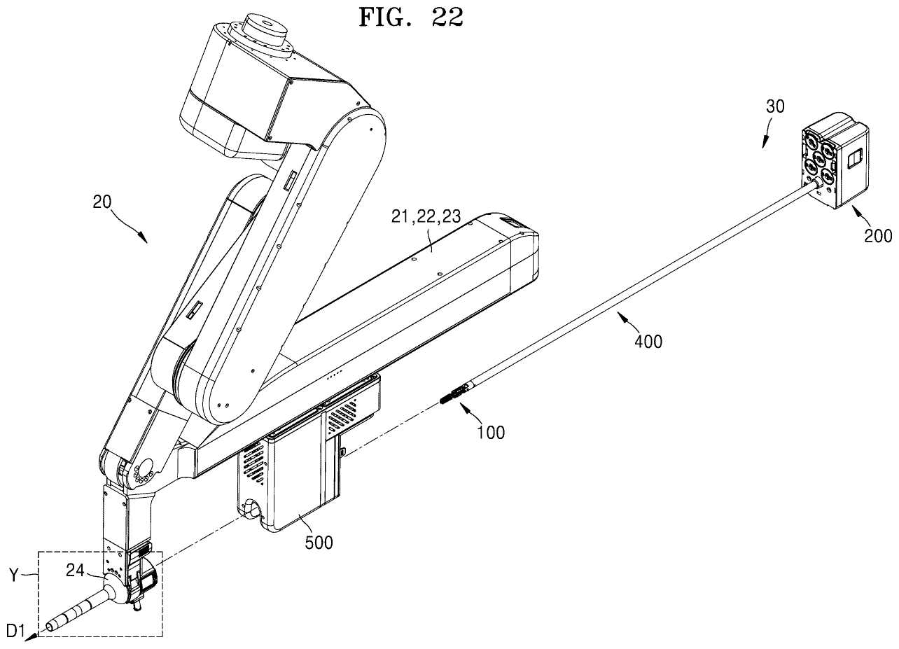

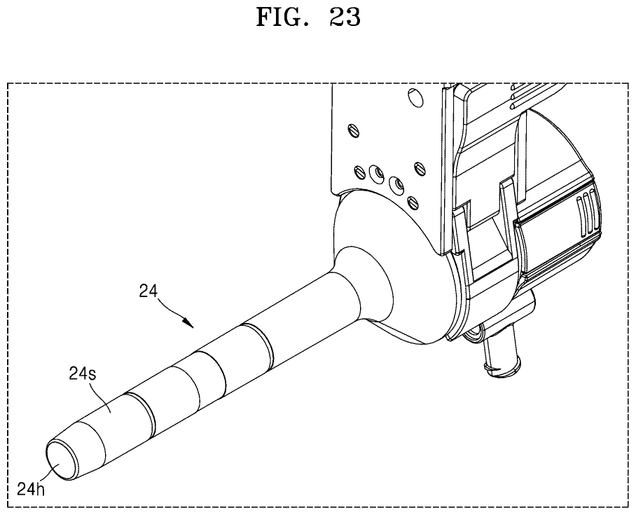

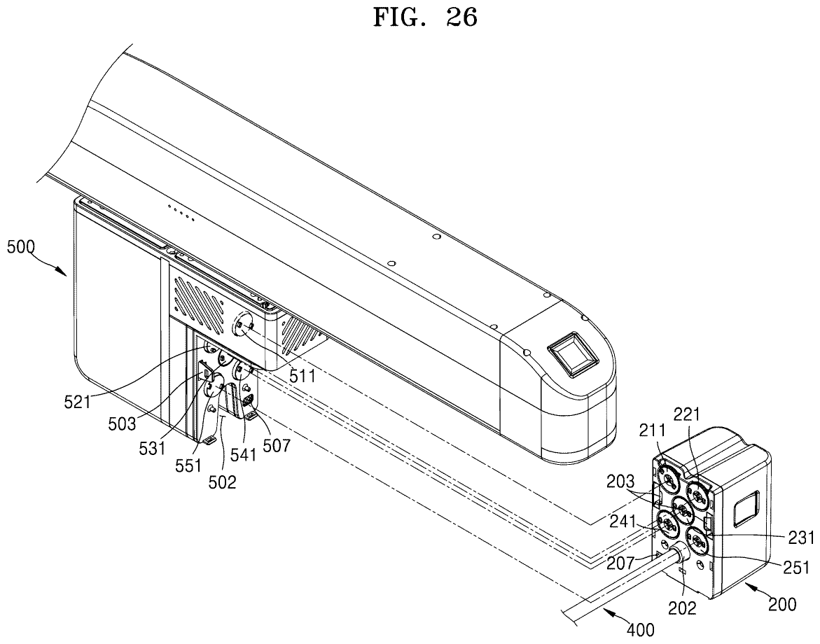

FIG. 22 is a diagram illustrating an example in which an instrument module is coupled to a slave robot, according to an embodiment of the present disclosure, and FIG. 23 is an enlarged view of portion Y of FIG. 22. FIG. 24 is a diagram illustrating a power transmission unit of FIG. 22, and FIG. 25 is a diagram illustrating an internal structure of the power transmission unit of FIG. 24. FIG. 26 is a diagram for describing a connection structure in which the instrument module of FIG. 22 is mounted on a slave robot.

Hereinafter, a connection structure in which the instrument module 30 according to an embodiment of the present disclosure is mounted on the robotic arm unit 21, 22, or 23 of the slave robot 20 will be described with reference to FIGS. 22 to 26.

The robotic arm unit 21, 22, or 23 may include a shaft accommodation part 24 and the power transmission unit 500.

The shaft accommodation part 24 may include a shaft through hole 24h through which the connection part 400 passes. The shaft accommodation part 24 may be formed to extend such that at least one area thereof has a length. The shaft through hole 24h may be formed along the extending area of the shaft accommodation part 24.

The shaft accommodation part 24 may surround at least a portion of the connection part 400 passing through the shaft through hole 24h. For example, the instrument module 30 may be mounted on the robotic arm unit 21, 22, or 23 such that the connection part 400 passes through the shaft through hole 24h.

Regarding a process of mounting the instrument module 30 on the robotic arm unit 21, 22, or 23 with reference to FIG. 22, the connection part 400 of the instrument module 30 may be inserted in a direction D1 to pass through the shaft through hole 24h of the shaft accommodation part 24. When the connection part 400 passes through the shaft through hole 24h to a certain degree, the driving part 200 may approach the power transmission unit 500, and the driving part 200 and the power transmission unit 500 may be fastened to each other. Accordingly, the instrument module 30 may be mounted on the robotic arm unit 21, 22, or 23 in a state in which the connection part 400 passes through the shaft through hole 24h.

The shaft accommodation part 24 may support the connection part 400 of the instrument module 30 mounted on the robotic arm unit 21, 22, or 23. Because the connection part 400 is formed to extend to have a length, the shaft accommodation part 24 supports and fixes the connection part 400 at a particular position such that the connection part 400 is not bent or directed in another direction. In detail, in a state in which the instrument module 30 is mounted on the robotic arm unit 21, 22, or 23, the connection part 400 may be in a direction intended by the user. As such, the shaft accommodation part 24 may intuitively match the direction in which the user wants to move the end tool 100, to the movement direction of the end tool 100.

The robotic arm units 21, 22, or 23 may include the power transmission unit 500 that generates a driving force for operating the instrument module 30, and transmits the generated driving force to the instrument. For example, the power transmission unit 500 may be connected to the driving part 200 and may transmit the driving force to the driving part 200.

For example, when the user manipulates the manipulation member 10a, a signal generated by the manipulation by the user may be transmitted to the power transmission unit 500, and the power transmission unit 500 may generate a driving force to correspond to the received signal, and transmit the driving force to the driving part 200.

The power transmission unit 500 may include a housing 501.

The housing 501 may be formed to protect at least a portion of the power transmission unit 500. For example, the housing 501 may be formed to protect a motor pack 560 and a control module 570, which will be described below.

The power transmission unit 500 may include the motor pack 560 and the control module 570.

The motor pack 560 may include at least one motor that generates a driving force for operating the instrument module 30 through manipulation by the user.

For example, the motor pack 560 may include a plurality of motors. In a specific embodiment, the motor pack 560 may include a first motor 561 that generates a driving force for operating the first jaw 101, a second motor 562 that generates a driving force for operating the second jaw 102, and a third motor 563 that generates a driving force for a pitch motion of the end tool 100.

The first motor 561 may generate a driving force for rotating the first jaw 101. For example, when the user inputs, to the manipulation member 10a, a signal for operating the first jaw 101 of the end tool 100, the first motor 561 may generate a driving force for rotating the first jaw 101.

The driving force generated by the first motor 561 may rotate the first jaw power transmission plate 511, which will be described below, and the first jaw power transmission plate 511 may rotate the first jaw 101 by rotating the first jaw driving member 211 of the driving part 200.

The second motor 562 may generate a driving force for rotating the second jaw 102. For example, when the user inputs, to the manipulation member 10a, a signal for operating the second jaw 102 of the end tool 100, the second motor 562 may generate a driving force for rotating the second jaw 102.

The driving force generated by the second motor 562 may rotate the second jaw power transmission plate 521, which will be described below, and the second jaw power transmission plate 521 may rotate the second jaw 102 by rotating the second jaw driving member 221 of the driving part 200.

The third motor 563 may generate a driving force for pitch-rotating the end tool 100. For example, when the user inputs, to the manipulation member 10a, a signal for pitch-rotating the end tool 100, the third motor 563 may generate a driving force for pitch-rotating the end tool 100.

The driving force generated by the third motor 563 may rotate the pitch power transmission plate 531, which will be described below, and the pitch power transmission plate 531 may pitch-rotate the end tool 100 by rotating the pitch driving member 231 of the driving part 200.

The motor pack 560 may further include a fourth motor 564 that generates a driving force for a roll motion of the instrument module 30.

The fourth motor 564 may generate a driving force for roll-rotating the connection part 400. For example, when the user inputs, to the manipulation member 10a, a signal for roll-rotating the end tool 100, the fourth motor 564 may generate a driving force for roll-rotating the end tool 100.

The driving force generated by the fourth motor 564 may rotate the roll power transmission plate 541, which will be described below, and the roll power transmission plate 541 may rotate the end tool 100 by rotating the instrument roll power transmission plate 241 of the driving part 200.

Optionally, the motor pack 560 may further include a fifth motor 565 that generates a driving force for moving the working member 140′ arranged in the instrument module 30.

The fifth motor 565 may generate a driving force for moving the working member 140′. For example, when the user inputs, to the manipulation member 10a, a signal for moving the working member 140′, the fifth motor 565 may generate a driving force for moving the working member 140′.

The driving force generated by the fifth motor 565 may rotate the translational power transmission plate 551, which will be described below, and translational power transmission plate 551 may move the working member 140′ by rotating the instrument translational power transmission plate 251 of the driving part 200.

The control module 570 may receive a signal generated by manipulation by the user, generate a signal for driving the motor pack 560 to correspond to the signal, and drive the motor pack 560 based on the generated signal. Alternatively, as described below, the control module 570 may store information such as whether the instrument module 30 is used, a usage history, the date of manufacture, the expiration date, and the like of the instrument module 30, which is received from the robot communication terminal 507, or process the information and deliver the processed information to the display member 10b to be provided to the user. However, the control module 570 is not limited thereto and may process various signals or information about driving of the instrument module 30.

The power transmission unit 500 may include at least one power transmission plate for transmitting a driving force received from the motor pack 560 to the driving part 200.

The power transmission plate may include the pitch power transmission plate 531 that transmits a driving force for controlling a pitch motion of the end tool 100, and a jaw power transmission plate that transmits a driving force for controlling a rotational motion of the jaw 103.

In detail, the power transmission unit 500 may include the first jaw power transmission plate 511 that transmits a driving force for controlling a rotational motion of the first jaw 101, and a second jaw power transmission plate 521 that transmits a driving force for controlling a rotational motion of the second jaw 102.

The first jaw power transmission plate 511 may be connected to the first motor 561. Thus, when the first motor 561 is driven, the first jaw power transmission plate 511 may rotate.

At least one first protrusion 511p may be formed on the first jaw power transmission plate 511. The first protrusion 511p is a portion inserted into the first insertion groove 211h formed on the first jaw driving member 211.

A plurality of first protrusions 51 1p may be formed, and it is preferable that the number of first protrusions 51 1p corresponds to the number of first insertion grooves 211h. As such, when a plurality of first protrusions 511p are respectively inserted into the first insertion grooves 211h, transmission of a driving force between the first jaw power transmission plate 511 and the first jaw driving member 211 may be efficiently performed.

The second jaw power transmission plate 521 may be connected to the second motor 562. Thus, when the second motor 562 is driven, the second jaw power transmission plate 521 may rotate.

At least one second protrusion 521p may be formed on the second jaw power transmission plate 521. The second protrusion 521p is a portion inserted into the second insertion groove 221h formed on the second jaw driving member 221.

A plurality of second protrusions 521p may be formed, and it is preferable that the number of second protrusions 521p corresponds to the number of second insertion grooves 221h. As such, when a plurality of second protrusions 521p are respectively inserted into the second insertion grooves 221h, transmission of a driving force between the second jaw power transmission plate 521 and the second jaw driving member 221 may be efficiently performed.

The pitch power transmission plate 531 may be connected to the third motor 563. Thus, when the third motor 563 is driven, the pitch power transmission plate 531 may rotate.

At least one third protrusion 531p may be formed on the pitch power transmission plate 531. The third protrusion 531p is a portion inserted into the third insertion groove 231h formed on the pitch driving member 231.

A plurality of third protrusions 531p may be formed, and it is preferable that the number of third protrusions 531p corresponds to the number of third insertion grooves 231h. As such, when a plurality of third protrusions 531p are respectively inserted into the third insertion grooves 231h, transmission of a driving force between the pitch power transmission plate 531 and the pitch driving member 231 may be efficiently performed.

The power transmission plate may include the roll power transmission plate 541 that transmits a driving force for controlling a roll rotation of the end tool 100.

The roll power transmission plate 541 may be connected to the fourth motor 564. Thus, when the fourth motor 564 is driven, the roll power transmission plate 541 may rotate.

At least one fourth protrusion 541p may be formed on the roll power transmission plate 541. The fourth protrusion 541p is a portion inserted into the fourth insertion groove 241h formed on the instrument roll power transmission plate 241.

A plurality of fourth protrusions 541p may be formed, and it is preferable that the number of fourth protrusions 541p corresponds to the number of fourth insertion grooves 241h. As such, when a plurality of fourth protrusions 541p are respectively inserted into the fourth insertion grooves 241h, transmission of a driving force between the roll power transmission plate 541 and the instrument roll power transmission plate 241 may be efficiently performed.

The power transmission plate may further include the translational power transmission plate 551 that transmits a driving force for control a movement of the working member 140′ of the end tool 100.

The translational power transmission plate 551 may be connected to the fifth motor 565. Thus, when the fifth motor 565 is driven, the translational power transmission plate 551 may rotate.

At least one fifth protrusion 551p may be formed on the translational power transmission plate 551. The fifth protrusion 55 1p is a portion inserted into the fifth insertion groove 251h formed on the instrument translational power transmission plate 251.

A plurality of fifth protrusions 55 1p may be formed, and it is preferable that the number of fifth protrusions 55 1p corresponds to the number of fifth insertion grooves 251h. As such, when a plurality of fifth protrusions 551p are respectively inserted into the fifth insertion grooves 251h, transmission of a driving force between the translational power transmission plate 551 and the instrument translational power transmission plate 251 may be efficiently performed.

A connection part accommodation groove 502 may be provided at a lower portion of the power transmission unit 500. For example, the connection part accommodation groove 502 may be formed to be concavely recessed from the outside to the inside of the power transmission unit 500. As another example, the connection part accommodation groove 502 may be formed in the shape of a hole to penetrate the power transmission unit 500 in one direction.

The connection part accommodation groove 502 is a portion that accommodates at least a portion of the connection part 400 when the instrument module 30 is mounted on the robotic arm unit 21, 22, or 23. For example, when the instrument module 30 is mounted on the robotic arm unit 21, 22, or 23, the connection part 400 may be arranged to pass, from the driving part 200, through the connection part accommodation groove 502 and the shaft through hole.

At least one first fastening part 503 may be formed in the power transmission unit 500. The first fastening part 503 may be inserted into the fastening groove 203 provided in the instrument module 30. By inserting and fixing the first fastening part 503 into the fastening groove 203, the instrument module 30 may be mounted and fixed on the robotic arm unit 21, 22, or 23 not to be separated.

At least one coupling ring 505 may be formed in the power transmission unit 500. The coupling ring 505 may be fastened to the coupling jaw 205 provided in the instrument module 30. Once the instrument module 30 is mounted on the robotic arm unit 21, 22, or 23 as the coupling ring 505 is caught and fixed on the coupling jaw 205, the instrument module 30 may remain mounted on the robotic arm unit 21, 22, or 23 unless the instrument module 30 is forcibly separated by manipulation by the user.

At least one insertion protrusion 506 may be formed on the power transmission unit 500. The insertion protrusion 506 may be inserted into the insertion groove 206 provided on the instrument module 30. The insertion protrusion 506 may be formed to extend outward to have a certain length. In this case, because the instrument module 30 is mounted on the robotic arm unit 21, 22, or 23 such that the insertion protrusion 506 is inserted into the insertion groove 206, the coupling between the instrument module 30 and the robotic arm unit 21, 22, or 23 may maintain consistency. In other words, the instrument module 30 may be mounted at a position where the insertion protrusion 506 is inserted into the insertion groove 206, that is, at a predetermined position of the robotic arm unit 21, 22, or 23 or the manipulation module 600.

Hereinafter, an embodiment in which the instrument module 30 is mounted on the robotic arm unit 21, 22, or 23 will be described with reference to FIG. 26.

The connection part 400 of the instrument module 30 is arranged to pass through the connection part accommodation groove 502 and then penetrate the shaft through hole. Then, the driving part 200 is connected to the power transmission unit 500. At this time, the first fastening part 503 is inserted into the fastening groove 203, the coupling ring 505 is caught on the coupling jaw 205, and the insertion protrusion 506 is inserted into the insertion groove 206. Through this, the instrument module 30 and the robotic arm unit 21, 22, or 23 may be coupled to each other at a designated position, and may not be unintentionally separated from each other after the coupling.

A driving member of the driving part 200 may be connected to a power transmission plate of the power transmission unit 500.

In detail, the first jaw driving member 211 may be connected to the first jaw power transmission plate 511. In addition, the second jaw driving member 221 may be connected to the second jaw power transmission plate 521. In addition, the pitch driving member 231 may be connected to the pitch power transmission plate 531. The instrument roll power transmission plate 241 may be connected to the roll power transmission plate 541. In addition, the instrument translational power transmission plate 251 may be connected to the translational power transmission plate 551.

As such, the instrument module 30 is mounted on the robotic arm unit 21, 22, or 23, and thus, the user may operate the end tool 100 by manipulating the surgical robot.

<Embodiment in which surgical instrument is mounted on manipulation module>

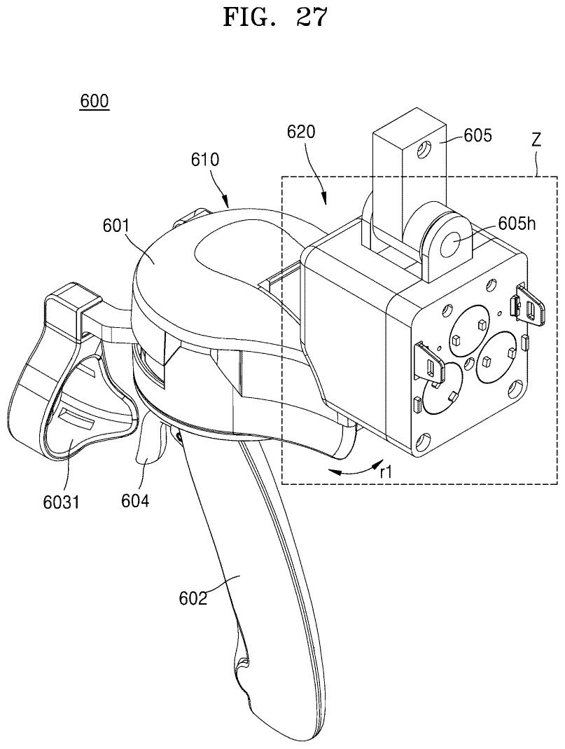

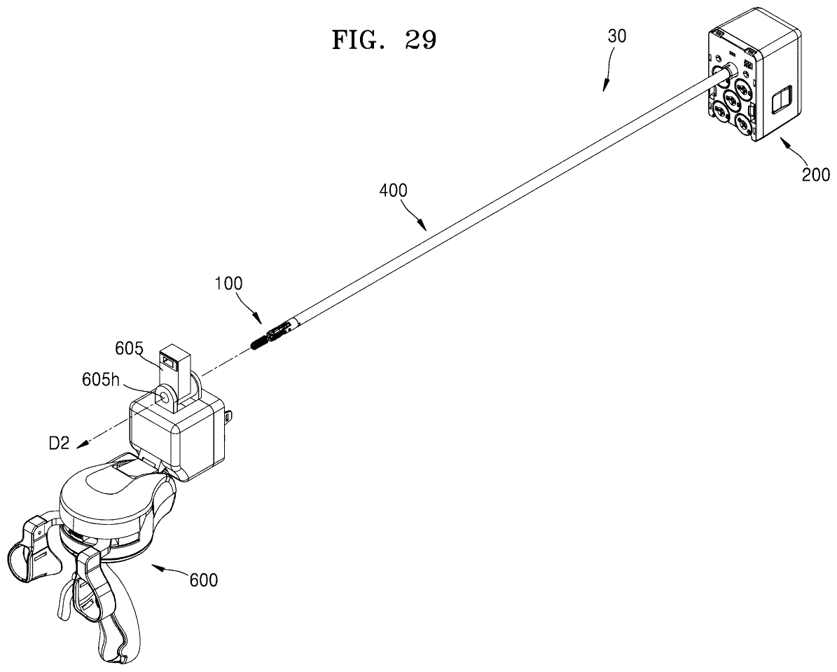

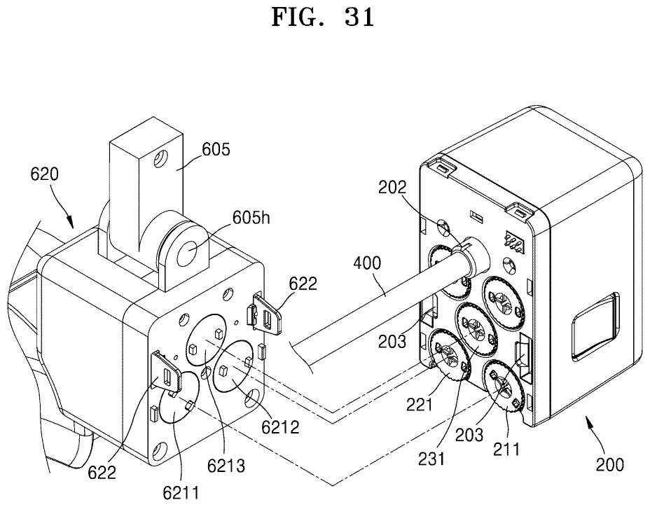

FIGS. 27 and 28 are diagrams illustrating a manipulation module according to an embodiment of the present disclosure, and FIG. 29 is a diagram illustrating a surgical instrument according to an embodiment of the present disclosure. FIG. 30 is an enlarged view of portion Z of FIG. 27, and FIG. 31 is a diagram for describing a connection structure in which the instrument module of FIG. 29 is coupled to a manipulation module.

As described above, the manipulation module 600 according to an embodiment of the present disclosure corresponds to a component that is detachably coupled to the instrument module 30 mounted and used on a surgical robot, so as to control a motion of the instrument module 30 mounted and used on the robot. That is, the manipulation module 600 according to an embodiment of the present disclosure is configured to be compatible with the instrument module 30 so as to improve the usability of the instrument module 30, and may be reused on the instrument module 30.

Thus, in order to achieve this purpose, the manipulation module 600 may include a component that functionally corresponds to a component included in a surgical robot (specifically, a power transmission unit of the surgical robot). In other words, just as the surgical robot may control a motion of the instrument module 30, the user may control a motion of the instrument module 30 by manipulating the manipulation module 600. However, the manipulation module 600 may not include all components corresponding to those included in the surgical robot, and some components may be omitted if necessary.

Hereinafter, the manipulation module 600 that may be mounted on the instrument module 30 mounted and used on a robot, a coupling structure between the instrument module 30 and the manipulation module 600 will be described in detail with reference to FIGS. 27 to 31.

Referring to FIGS. 27 to 31, the manipulation module 600 according to an embodiment of the present disclosure may be detachably coupled to the instrument module 30.

The manipulation module 600 is a device through which the user performs various manipulations to cause the instrument module 30 to perform various motions. In an embodiment, the manipulation module 600 may be formed as a hand-held type. In other words, it may be described that the manipulation module 600 is a device for allowing a user to manually manipulate the instrument module 30.

The manipulation module 600 may include a manipulation part 610 and a power connection part 620.

The manipulation part 610 may be formed to enable manipulation by the user to control a motion of the end tool 100. For example, the user may manipulate the manipulation part 610 to provide the manipulation part 610 with a driving force for operating the end tool 100. The driving force generated by manipulation by the user may be transmitted to the instrument module 30 through the power connection part 620, which will be described below.

The manipulation part 610 may include a handle 602 that the user may hold, and the actuation manipulation part 603 that controls an actuation motion of the end tool 100.

The handle 602 may be formed to be held by the user. In particular, the handle 602 may be formed such that the user may hold it by wrapping it with his or her palm. For example, the handle 602 may be formed in the form of a bar that extends to have a length.

In an embodiment, the handle 602 may be formed to be rotatable with respect to the power connection part 620 by manipulation by the user. For example, the handle 602 may be relatively rotatable in the left and right directions with respect to the power connection part 620.

In detail, as illustrated in FIG. 27, the manipulation part 610 may be divided into two or more parts that rotate with respect to each other, a handle may be formed on or coupled to one of the parts, and the other part may be coupled to the power connection part 620. Thus, when the user holds and rotates the handle 602, the handle 602 and the portion where the handle 602 is arranged may rotate with respect to the power connection part 620. As such, a rotational motion of the handle 602 is associated with a yaw motion of the end tool 100. This will be described in detail below.

The manipulation part 610 may include the actuation manipulation part 603. The actuation manipulation part 603 may be formed to enable manipulation for controlling an actuation motion of the jaw 103 of the end tool 100.

In an embodiment, the actuation manipulation part 603 may be provided to correspond to a pair of jaws 101 and 102 in a one-to-one manner. For example, the actuation manipulation part 603 may include the first actuation manipulation part 6031 and the second actuation manipulation part 6032. The first actuation manipulation part 6031 is associated with a motion of the first jaw 101 of the end tool 100, and the second actuation manipulation part 6032 is associated with a motion of the second jaw 102 of the end tool 100. In an alternative embodiment, the first actuation manipulation part 6031 and the second actuation manipulation part 6032 may be formed in the shape of a ring into which the user may insert a finger, but are not limited thereto.

The actuation manipulation part 603 may be arranged on both sides of the front of the handle 602. For example, the actuation manipulation part 603 may be arranged closer to the end tool 100 than the handle 602. This is for ergonomic design and is suitable for the structure of the hand of the user holding the actuation manipulation part 603. Thus, the user may more easily manipulate the actuation manipulation part 603 while holding the handle 602.

The manipulation module 600 may include the power connection part 620.

The power connection part 620 may transmit, to the instrument module 30, a driving force generated as the user manipulates the manipulation part 610. For example, when the user manipulates the manipulation part 610 to operate the instrument module 30, a driving force for operating the instrument module 30 may be generated by the manipulation part 610 due to the manipulation, and the power connection part 620 may transmit the generated driving force from the manipulation part 610 to the driving part 200 of the instrument module 30.

The power connection part 620 may be connected to one side of the manipulation part 610. For example, as illustrated in FIG. 28, the power connection part 620 may be connected to a rear upper portion of the manipulation part 610. Accordingly, the instrument module 30 may be coupled to the rear of the manipulation part 610 toward the front. In other words, the instrument module 30 may be connected while moving from the power connection part 620 toward the manipulation part 610. This will be described in detail below.

The power connection part 620 may be detachably coupled to the instrument module 30. In detail, the power connection part 620 may be detachably coupled to the driving part 200 of the instrument module 30. In other words, it may also be described that the power connection part 620 is arranged between the manipulation part 610 and the driving part 200 to transmit a driving force generated by the manipulation part 610 to the driving part 200. In other words, it may also be described that the power connection part 620 may connect the manipulation module 600 to the instrument module 30 and thus enable power transmission.

The manipulation part 610 and the power connection part 620 may be formed to be rotatable with respect to each other. For example, the manipulation part 610 may rotate with respect to the power connection part 620.

For example, the manipulation part 610 may be rotatable in the upward and downward directions with respect to the power connection part 620. To this end, the manipulation part 610 and the power connection part 620 may be coupled to rotatable around one axis, and preferably, may be hinge-coupled to each other.

The relative rotation of the manipulation part 610 and the power connection part 620 in the upward or downward directions is associated with a pitch motion of the end tool 100. In other words, because the manipulation part 610 and the power connection part 620 may rotate in the upward or downward directions with respect to each other, the user enables a pitch motion of the end tool 100 by rotating the manipulation part 610 in the upward or downward direction with respect to the power connection part 620, and this enables intuitive operation of the instrument module 30.

The manipulation module 600 may include a housing 601. The housing 601 may form the exterior of the power connection part 620.

For example, the housing 601 may include a first housing 601a and a second housing 601b. The first housing 601a may form the exterior of the manipulation part 610. The second housing 601b may form the exterior of the power connection part 620.

In an alternative embodiment, the manipulation module 600 may further include a working member manipulation part 604. The working member manipulation part 604 is associated with driving of a motion of the working member 140′ provided in the end tool 100′. For example, the user may move the working member 140′ or limit the movement of the working member 140′ by manipulating the working member manipulation part 604.

The working member manipulation part 604 may be arranged at a position where the user may easily manipulate it while holding the handle 602. For example, the working member manipulation part 604 may be arranged in an area adjacent to the handle 602. In this case, the user may easily manipulate the working member manipulation part 604 with his or her index or middle finger while holding the handle 602. However, the working member manipulation part 604 is not limited thereto, and may be arranged on a side surface or an upper surface of the manipulation module 600 such that the user may easily manipulate it with his or her thumb.

The working member manipulation part 604 may be formed in a lever manner that the user pulls with his or her finger, or a button manner, but is not limited thereto and may be implemented in various manners.

The manipulation module 600 may further include a mounting part 605. The mounting part 605 is arranged at one position of the manipulation module 600 and is used to mount the manipulation module 600 at a particular position. For example, there is a case in which the user performing surgery needs to release one or both hands from the manipulation module 600 during the surgery, and the mounting part 605 may allow the manipulation module 600 to be mounted at a particular position in such cases. In other words, the manipulation module 600 may be mounted on the instrument module 30 in a state in which the mounting part 605 is mounted at a particular position in an operating room, a particular structure, device, or surgical robot, or the like.

A guide hole 605h may be formed at an upper portion of the second housing 601b to guide the coupling direction of the instrument module 30. The shaft-shaped connection part 400 provided in the instrument module 30 may be inserted into the guide hole 605h.

For example, a separate member having the guide hole 605h formed thereon may be mounted on an upper portion of the second housing 601b, or the guide hole 605h may be integrally formed on the second housing 601b.

A through hole may be formed on the mounting part 605, and the mounting part 605 may be coupled to an upper portion of the second housing 601b such that the through hole and the guide hole 605h form one through hole. In other words, the connection part 400 may be mounted on the manipulation module 600 to simultaneously pass through the guide hole 605h and the through hole of the mounting part 605.

Regarding a process of mounting the instrument module 30 on the manipulation module 600 with reference to FIG. 29, the connection part 400 of the instrument module 30 may be inserted in a direction D2 to simultaneously pass through the guide hole 605h and the through hole of the mounting part 605. When the connection part 400 passes through the guide hole 605h and the hole of the mounting part 605 to a certain degree, the driving part 200 may approach the power connection part 620, and the driving part 200 and the power connection part 620 may be coupled to each other. Accordingly, the instrument module 30 may be mounted on the manipulation module 600 in a state in which the connection part 400 passes through the guide hole 605h and the through hole of the mounting part 605. That is, the guide hole 605h may guide the coupling direction of the instrument module 30 and the manipulation module 600.

The guide hole 605h and the through hole of the mounting part 605 may support the connection part 400 of the instrument module 30 mounted on the manipulation module 600. Because the connection part 400 is formed to extend to have a length, the guide hole 605h and the through hole of the mounting part 605 support and fix the connection part 400 at a particular position such that the connection part 400 is not bent or directed in another direction. In detail, in a state in which the instrument module 30 is mounted on the manipulation module 600, the connection part 400 may be in a direction intended by the user.

In addition, the guide hole 605h and the through hole of the mounting part 605 may also serve to fix the connection part 400 at a particular position such that the instrument module 30 may roll-rotate around the connection part 400. As described below, when the instrument module 30 is connected to the manipulation module 600, a roll motion of the instrument module 30 may be implemented by the user holding and roll-rotating the manipulation module 600. In this case, the guide hole 605h and the through hole of the mounting part 605 may fix the connection part 400 at a particular position such that the instrument module 30 may rotate around the connection part 400, and roll rotation of the instrument module 30 may be performed stably.

The power connection part 620 may include a shape and structure that allows the instrument module 30 to be mounted on the manipulation module 600.

For example, at least one second fastening part 622 may be formed in the power connection part 620. The second fastening part 622 may be inserted into the fastening groove 203 provided in the instrument module 30. By inserting and fixing the second fastening part 622 into the fastening groove 203, the instrument module 30 may be mounted and fixed on the manipulation module 600 not to be separated.

The manipulation module 600 may include a component for transmitting a driving force generated by manipulation by the user to the driving part 200. In detail, the power connection part 620 may include a component that is connected to the driving part 200 to transmit a driving force to the driving part 200.

In an embodiment, the power connection part 620 may include a coupling area. The coupling area may refer to a portion where the power connection part 620 and the driving part 200 are coupled to each other. The coupling area may include a coupling surface that is in contact with and connected to the driving part 200, as illustrated in FIG. 31 and the like.

A driving force generated by manipulation by the user may be transmitted in the coupling area. In other words, it may also be described that connection for transmission of a driving force is implemented as the power connection part 620 and the driving part 200 are coupled to each other in the coupling area. In other words, it may also be described that the coupling area is a portion where the power connection part 620 is connected to an interface for transmitting a driving force of the driving part 200.

The power connection part 620 may include at least one power transmission member.

The power transmission member is a member that performs a particular motion when a driving force is generated from the manipulation module 600 by manipulation by the user. Here, the power transmission member may be engaged with the driving member of the driving part 200 as will be described below, and the driving member may also perform a motion due to a motion of the power transmission member.

In a specific embodiment, the power transmission member may perform a rotational motion through manipulation by the user. In this case, as the power transmission member rotates, the driving member may rotate together with the power transmission member, and the rotation of the driving member may cause motions of the pulleys and the wires arranged inside the driving part 200, and thus control a motion of the end tool 100.

In an embodiment, the power transmission member may be arranged such that at least a portion of the power transmission member overlaps the coupling area. In other words, as illustrated in FIG. 31 and the like, at least a portion of the power transmission member may be exposed to the coupling surface of the power connection part 620 connected to the driving part 200. Accordingly, the power connection part 620 and the driving part 200 may be connected to each other to enable transmission of a driving force through the mechanical coupling of the power connection part 620 and the driving part 200.

Here, the driving force generated by manipulation by the user does not refer to a force generated by using a motor or the like, but may refer to a driving force applied by the user to move a particular member of the manipulation module 600 (e.g., the handle 602 or the actuation manipulation part 603) or to rotate the manipulation module 600 itself. In other words, it may also be described that, when the user applies a force to the manipulation module 600, the force is transmitted as a rotational motion of the power transmission member through a plurality of pulleys and wires provided in the manipulation module 600 as will be described below, and thus, the force applied by the user to the manipulation module 600 serve as the driving force.

The power transmission member may include the pitch power transmission member 6213 that transmits a driving force for controlling a pitch motion of the end tool 100, and the jaw power transmission members 6211 and 6212 that transmit a driving force for controlling a rotational motion of the jaw 103.

In detail, the power connection part 620 may include the first jaw power transmission member 6211 that transmits a driving force for controlling a rotational motion of the first jaw 101, and the second jaw power transmission member 6212 that transmits a driving force for controlling a rotational motion of the second jaw 102.

In an embodiment, the first jaw power transmission member 6211 and the second jaw power transmission member 6212 may be formed to operate independently of each other. For example, the first jaw power transmission member 6211 and the second jaw power transmission member 6212 may rotate in the same direction or in different directions. This means that the first jaw power transmission member 6211 and the second jaw power transmission member 6212 may operate independently of each other, and accordingly, the first jaw 101 and the second jaw 102 may also rotate in the same direction or in different directions. Accordingly, the first jaw 101 and the second jaw 102 may move toward each other to perform an actuation motion, or move in the same direction to perform a yaw motion of the end tool 100.

As described below, the jaw power transmission members 6211 and 6212 and the pitch power transmission member 6213 may be connected to the pulleys arranged inside the power connection part 620. Thus, when the pulleys are rotated by manipulation by the user, the jaw power transmission members 6211 and 6212 and the pitch power transmission member 6213, which are connected to the pulleys, may rotate together.

At least one 1-1st protrusion 6211p may be formed on the first jaw power transmission member 6211. The 1-1st protrusion 621 1p is a portion inserted into the first insertion groove 211h formed on the first jaw driving member 211.

A plurality of 1-1st protrusions 621 1p may be formed, and it is preferable that the number of 1-1st protrusions 6211p corresponds to the number of first insertion grooves 211h. As such, when a plurality of 1-1st protrusions 621 1p are respectively inserted into the first insertion grooves 211h, transmission of a driving force between the first jaw power transmission member 6211 and the first jaw driving member 211 may be efficiently performed.

At least one 2-1st protrusion 6212p may be formed on the second jaw power transmission member 6212. The 2-1st protrusion 6212p is a portion inserted into the second insertion groove 221h formed on the second jaw driving member 221.

A plurality of 2-1st protrusions 6212p may be formed, and it is preferable that the number of 2-1 st protrusions 6212p corresponds to the number of second insertion grooves 221h. As such, when a plurality of 2-1st protrusions 6212p are respectively inserted into the second insertion grooves 221h, transmission of a driving force between the second jaw power transmission member 6212 and the second jaw driving member 221 may be efficiently performed.

At least one 3-1st protrusion 6213p may be formed on the pitch power transmission member 6213. The 3-1st protrusion 6213p is a portion inserted into the third insertion groove 231h formed on the pitch driving member 231.

A plurality of 3-1st protrusions 6213p may be formed, and it is preferable that the number of 3-1st protrusions 6213p corresponds to the number of third insertion grooves 231h. As such, when a plurality of 3-1st protrusions 6213p are respectively inserted into the third insertion grooves 231h, transmission of a driving force between the pitch power transmission member 6213 and the pitch driving member 231 may be efficiently performed.

Referring to FIG. 31, the driving part 200 and the power connection part 620 may be connected to each other. In detail, the power transmission members 6211, 6212, and 6213 of the power connection part 620 may be coupled to the driving members 211, 221, and 231 of the driving part 200 in a one-to-one correspondence.

For example, the first jaw driving member 211 may be connected to the first jaw power transmission member 6211. In addition, the second jaw driving member 221 may be connected to the second jaw power transmission member 6212. In addition, the pitch driving member 231 may be connected to the pitch power transmission member 6213.

Thus, rotation of the first jaw power transmission member 6211 may cause only rotation of the first jaw driving member 211, rotation of the second jaw power transmission member 6212 may cause only rotation of the second jaw driving member 221, and rotation of the pitch power transmission member 6213 may cause only rotation of the pitch driving member 231.

Accordingly, when the user manipulates the manipulation module 600 for an actuation motion, a pitch motion, and a yaw motion of the end tool 100, the manipulation actions may be performed independently of each other, and may not affect each other.

As such, the instrument module 30 is mounted on the manipulation module 600, and thus, the user may operate the end tool 100 by manipulating the manipulation module 600.

### <Intuitive Operation>

As described above, the instrument module 30 according to the present disclosure may be mounted and used on the manipulation module 600. In this case, a motion of the instrument module 30 may intuitively match a motion of the manipulation module 600. Hereinafter, an intuitive operation of the instrument module 30 of the present disclosure will be described.

In order to implement an intuitive operation of the instrument module 30, the manipulation module 600 may be formed to be rotatable in at least two directions. For example, as illustrated in FIG. 27, the manipulation module 600 may be formed such that the manipulation part 610 is rotatable in a direction r1 with respect to the power connection part 620. In addition, as illustrated in FIG. 28, the manipulation part may be formed such that at least one area of the manipulation module 600 is rotatable in a direction r2, and for example, the handle 602 and a portion connected to the handle 602 may be formed to be rotatable in the direction r2.

Furthermore, in order to implement an intuitive operation of the instrument module 30, the actuation manipulation part 603 may include the actuation manipulation parts 6031 and 6032 that correspond to a pair of jaws 101 and 102 in a one-to-one manner, and the pair of jaws 101 and 102 may be formed to be closed together when the actuation manipulation parts 6031 and 6032 are closed.

Referring back to FIGS. 27 and 28, the user may perform a pitch motion by rotating the handle 602 in the direction r1 while holding the handle 602 with the palm of his or her hand, and may perform a yaw motion by rotating the handle 602 in the direction r2. In addition, the user may perform an actuation motion of the end tool 100 by, manipulating the actuation manipulation part 603 to be closed in a state the thumb and index finger are inserted into the first actuation manipulation part 6031 and the second actuation manipulation part 6032 formed at one end of the actuation manipulation part 603.

Here, in the instrument module 30 according to the present disclosure, when the manipulation module 600 is rotated in any one direction with respect to the connection part 400, the end tool 100 rotates in the intuitively same direction as the manipulation direction of the manipulation module 600. In other words, when the handle 602 of the manipulation module 600 rotates in any one direction, the end tool 100 also rotates in the intuitively same direction as the one direction, such that a pitch motion or a yaw motion is performed. Here, the intuitively same direction may indicate that the movement direction of a finger of the user holding the manipulation module 600 is substantially the same as the movement direction of the distal end of the end tool 100. Obviously, “the same direction” as used herein may not be a perfectly matching direction on a three-dimensional coordinate, and may be understood to be equivalent to the extent that, for example, when the user's finger moves to the left, the distal end of the end tool 100 is moved to the left, and when the user's finger moves downward, the end portion of the end tool 100 is moved downward.

In addition, to this end, in the instrument module 30 according to the present embodiment, the manipulation module 600 and the end tool 100 are formed in the same direction with respect to a plane perpendicular to the extension axis of the connection part 400, in a state in which the instrument module 30 is mounted on the manipulation module 600. That is, in FIG. 2, the manipulation module 600 is formed to extend in the left direction, and the end tool 100 is also formed to extend in the positive (+) X-axis direction. In other words, it may also be described that the formation direction of the end tool 100 on one end of the connection part 400 is the same as the formation direction of the manipulation module 600 on the other end of the connection part 400, with respect to a plane perpendicular to the extension axis of the connection part 400. Alternatively, in other words, it may also be described that the manipulation module 600 is formed in a direction away from the body of the user holding the manipulation module 600, that is, in the direction in which the end tool 100 is formed. That is, in the handle 602 or the like that is held and moved by the user for an actuation motion, a yaw motion, and a pitch motion, a part that moves to perform each motion extends in the positive (+) X-axis direction beyond the center of rotation of each joint for the corresponding motion. Through this, the manipulation module 600 may be configured in the same manner as the end tool 100 in which each moving part is formed to extend in the positive (+) X-axis direction from the center of rotation of a corresponding joint for the motion, the manipulation direction of the user may be identical to the motion direction of the end tool from the viewpoint of the rotation directions and the left and right directions, and accordingly, the intuitively same manipulation may be possible.

In detail, in a related-art surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to a patient.

To overcome such issue, in the instrument module 30 according to the present disclosure, the manipulation direction of the manipulation module 600 may be intuitively identical to the operation direction of the end tool 100, and to this end, a portion of the manipulation module 600 which actually moves for an actuation motion, a yaw motion, and a pitch motion may extend in the positive (+) X-axis direction beyond the center of rotation of a joint for the respective motions as in the end tool 100.

Hereinafter, an intuitive operation of the instrument module 30 by the manipulation module 600 will be described in detail.

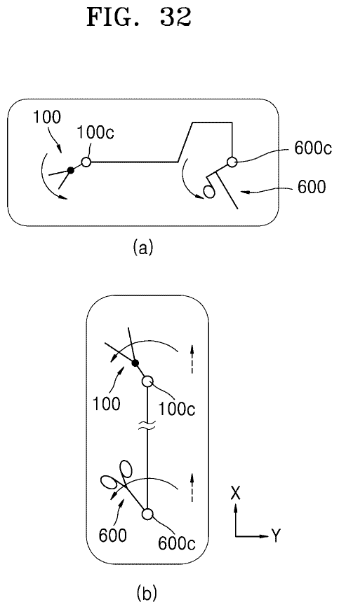

FIG. 32 is a conceptual diagram for describing an operation of the surgical instrument of FIG. 3.

In detail, referring to (a) and (b) of FIG. 32, in a surgical instrument of the present embodiment, the end tool 100 is formed in front of a center of rotation 100c of the end tool, and the manipulation module 600 is also formed in front of a center of rotation 600c of the manipulation module 600, such that the operations of the manipulation module 600 and the end tool 100 intuitively match. To express these characteristics differently, the instrument module 30 according to an embodiment of the present disclosure is formed such that at least a portion of the manipulation module 600 may be closer to the end tool 100 (than its own joint) with respect to its own joint at more than one point in a manipulation process.

FIGS. 33 to 36 are perspective views illustrating a pitch motion of the surgical instrument of FIG. 3.

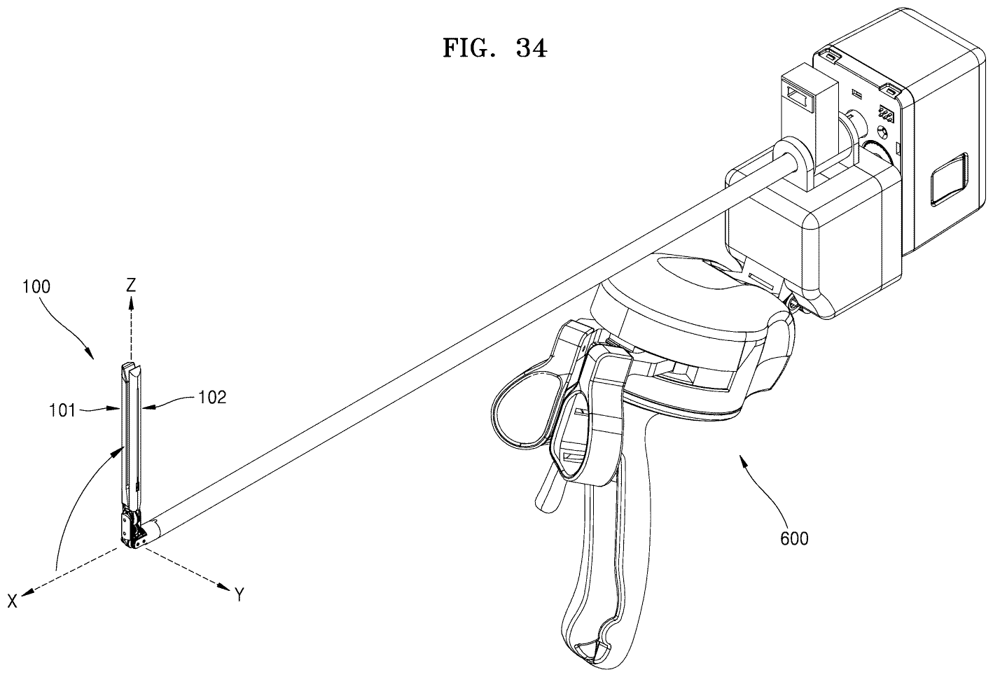

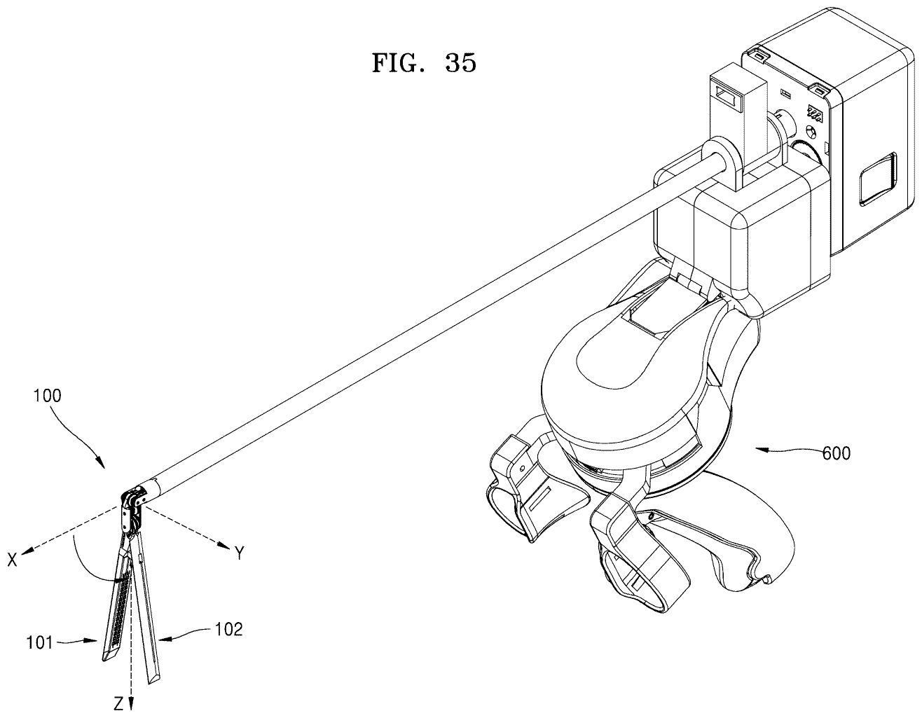

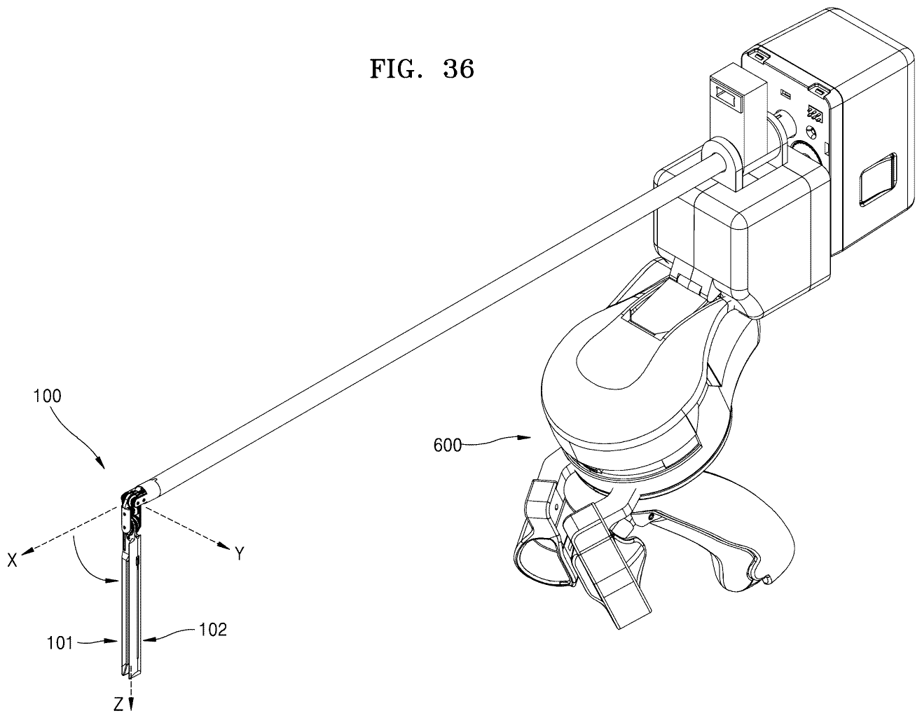

In detail, FIG. 33 is a diagram illustrating a state in which jaws are pitch-rotated by −90°, and FIG. 34 is a diagram illustrating a process of performing an actuation motion in a state in which the jaws 101 and 102 are pitch-rotated by −90°. FIG. 35 is a diagram illustrating a state in which jaws are pitch-rotated by +90°, and FIG. 36 is a diagram illustrating a process of performing an actuation motion in a state in which jaws are pitch-rotated by +90°.

Referring to FIGS. 33 to 36, it may be seen that, in performing a pitch motion, the motions of the manipulation module 600 and the end tool 100 intuitively match. That is, when the manipulation module 600 rotates in a positive (+) direction with respect to the pitch rotation shaft (Y-axis), the end tool 100 also rotates in the positive (+) direction with respect to the pitch rotation shaft (Y-axis). In addition, when the manipulation module 600 rotates in a negative (−) direction with respect to the pitch rotation shaft (Y-axis), the end tool 100 also rotates in the negative (−) direction with respect to the pitch rotation shaft (Y-axis). Here, the rotation angle of the manipulation module 600 and the rotation angle of the end tool 100 may be variously set according to the proportions of the pulleys. In addition, when the actuation manipulation part is closed in a state in which the manipulation module 600 rotates in the positive (+) or negative (−) direction with respect to the pitch rotation shaft (Y-axis), the jaws 101 and 102 are also closed to perform an actuation motion.

FIGS. 37 to 40 are perspective views illustrating a yaw motion of the surgical instrument of FIG. 3.

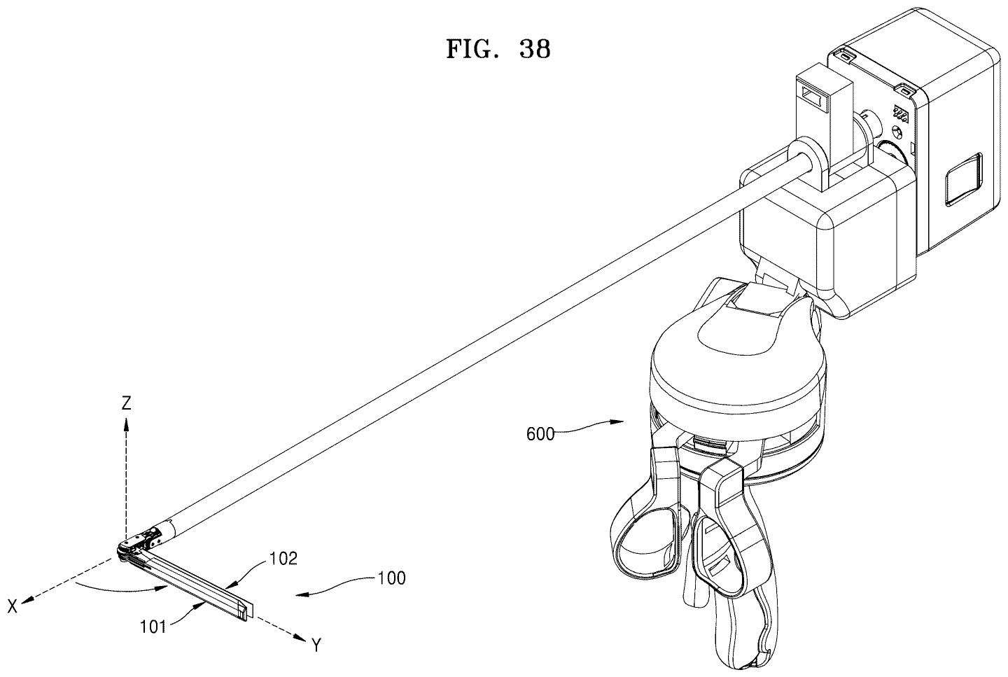

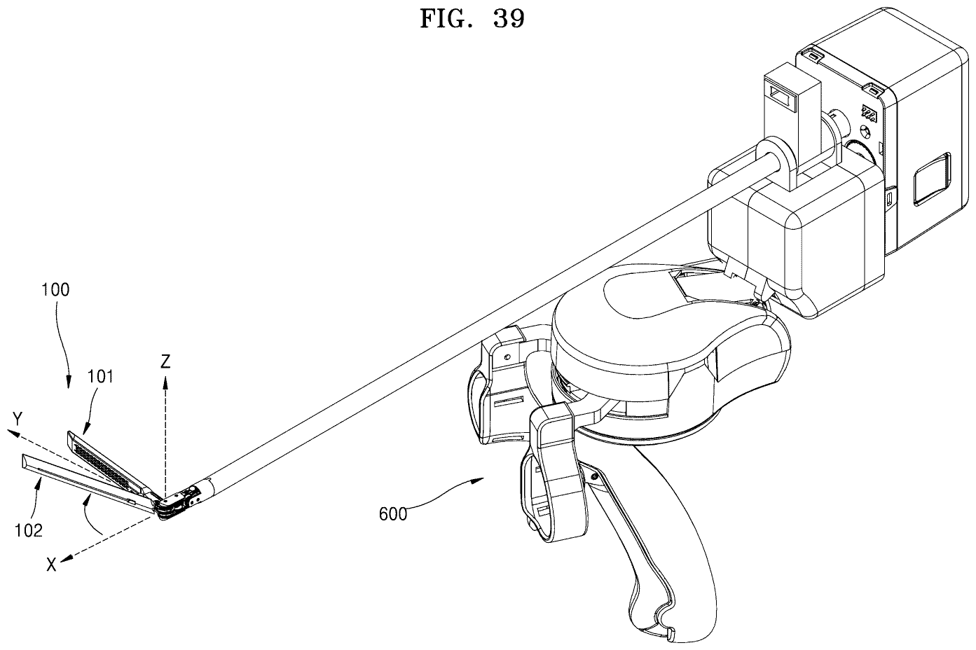

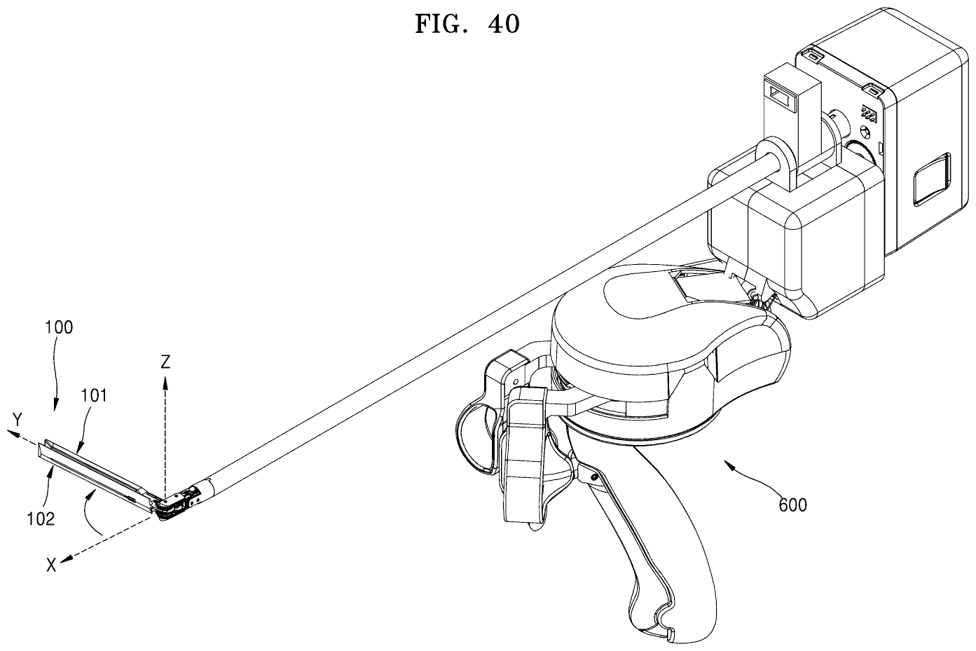

In detail, FIG. 37 is a diagram illustrating a state in which jaws are yaw-rotated by +90°, and FIG. 38 is a diagram illustrating a process of performing an actuation motion in a state in which the jaws 101 and 102 are yaw-rotated by +90°. FIG. 39 is a diagram illustrating a state in which jaws are yaw-rotated by −90°, and FIG. 40 is a diagram illustrating a process of performing an actuation motion in a state in which jaws are yaw-rotated by −90°.

Referring to FIGS. 37 to 40, it may be seen that, in performing a yaw motion, the motions of the manipulation module 600 and the end tool 100 intuitively match. That is, when the manipulation module 600 rotates in a positive (+) direction with respect to the yaw rotation shaft (Z-axis), the end tool 100 also rotates in the positive (+) direction with respect to the yaw rotation shaft (Z-axis). In addition, when the manipulation module 600 rotates in a negative (+) direction with respect to the yaw rotation shaft (Z-axis), the end tool 100 also rotates in the negative (−) direction with respect to the yaw rotation shaft (Z-axis). Here, the rotation angle of the manipulation module 600 and the rotation angle of the end tool 100 may be variously set according to the proportions of the pulleys. In addition, when the actuation manipulation part is closed in a state in which the manipulation module 600 rotates in the positive (+) or negative (−) direction with respect to the yaw rotation shaft (Z-axis), the jaws 101 and 102 are also closed to perform an actuation motion.

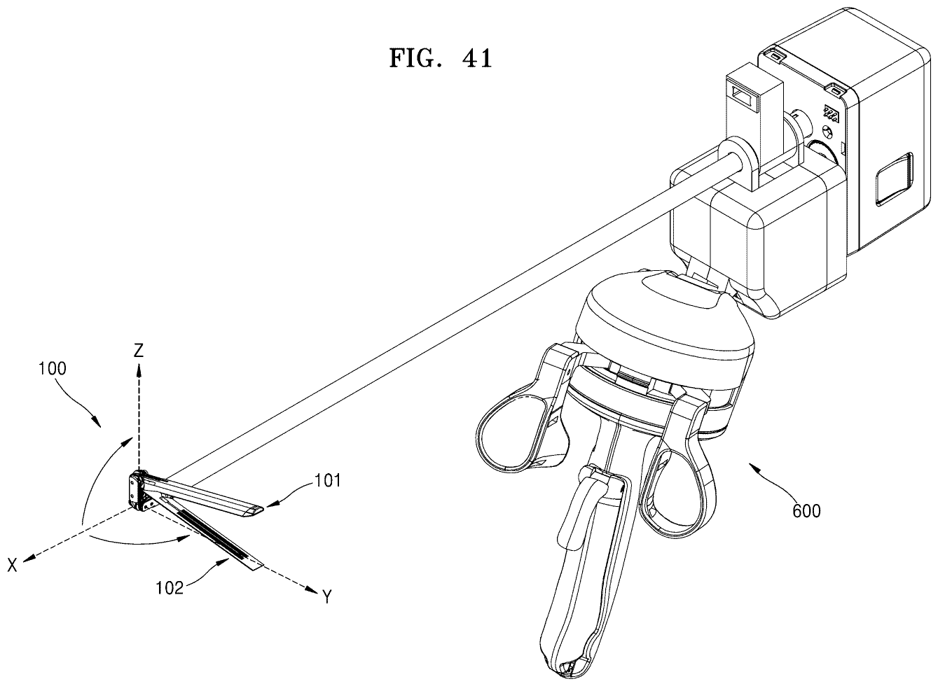

FIGS. 41 to 44 are plan views illustrating a state in which the end tool for a surgical instrument of FIG. 1 is pitch-rotated and yaw-rotated.

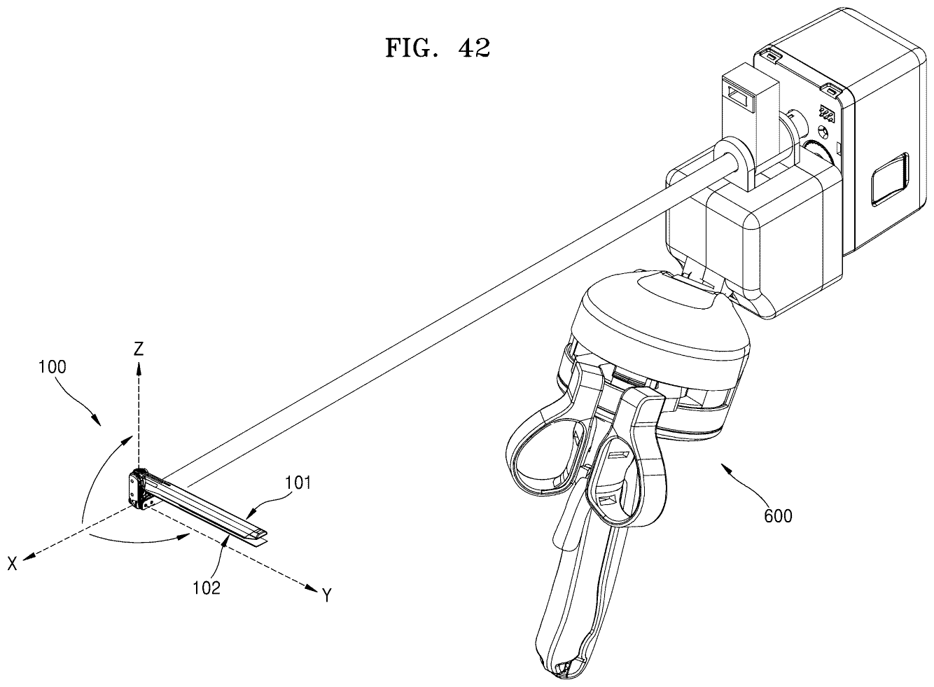

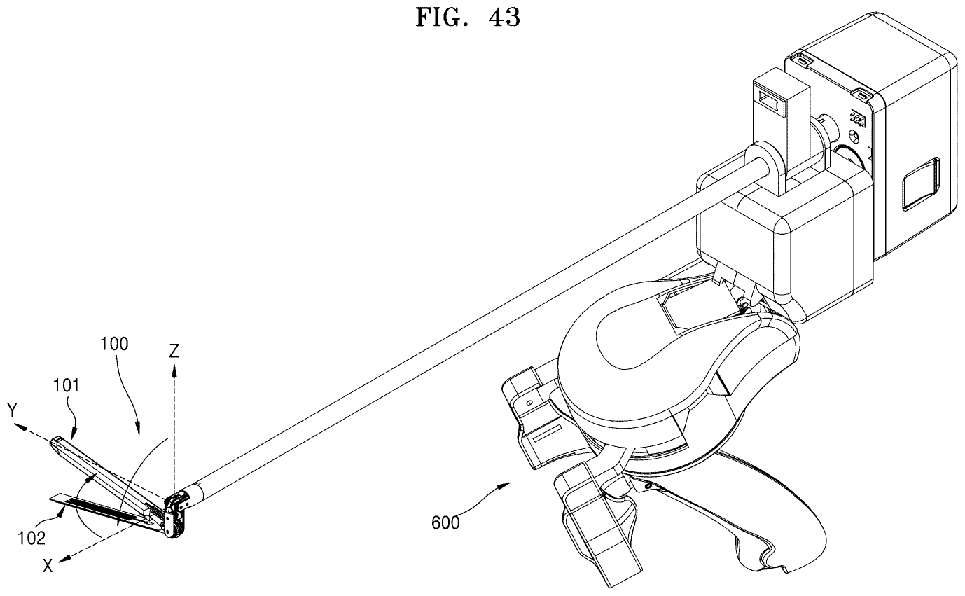

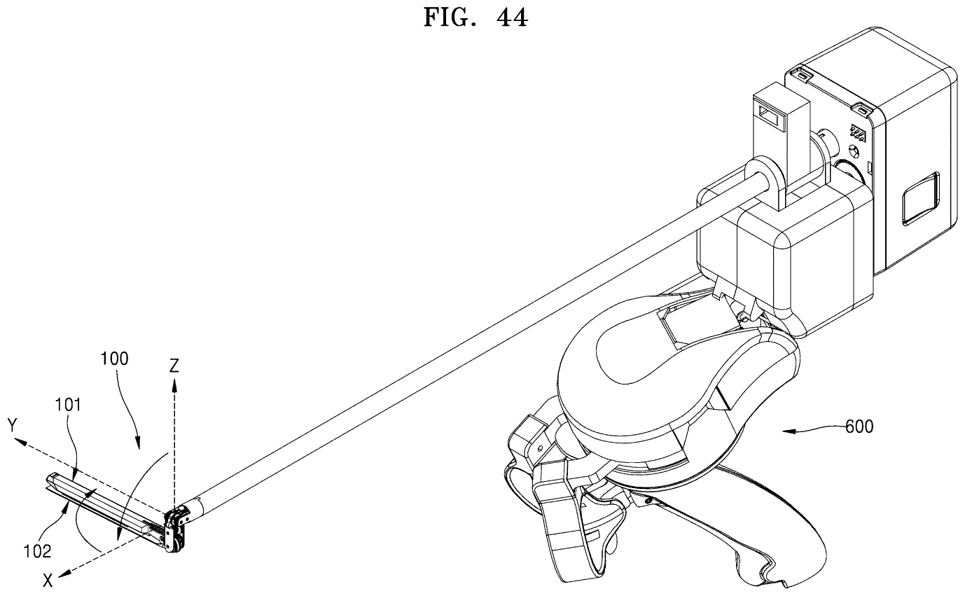

FIG. 41 is a diagram illustrating a state in which jaws are pitch-rotated by −90° and simultaneously yaw-rotated by +90°, and FIG. 42 is a diagram illustrating a process of performing an actuation motion in a state in which jaws are pitch-rotated by −90° and simultaneously yaw-rotated by +90°. FIG. 43 is a diagram illustrating a state in which jaws are pitch-rotated by +90° and simultaneously yaw-rotated by −90°, and FIG. 44 is a diagram illustrating a process of performing an actuation motion in a state in which jaws are pitch-rotated by +90° and simultaneously yaw-rotated by −90°.

Referring to FIGS. 41 to 44, it may be seen that, in performing a pitch motion and a yaw motion, the motions of the manipulation module 600 and the end tool 100 intuitively match. That is, when the manipulation module 600 rotates in the negative (−) direction with respect to the pitch rotation shaft (Y-axis), and rotates in the positive (+) direction with respect to the yaw rotation shaft (Z-axis), the end tool 100 also rotates in the negative (−) direction with respect to the pitch rotation axis (Y-axis) and rotates in the positive (+) direction with respect to the yaw rotation axis (Z-axis). In addition, when the manipulation module 600 rotates in the positive (+) direction with respect to the pitch rotation shaft (Y-axis), and rotates in the negative (−) direction with respect to the yaw rotation shaft (Z-axis), the end tool 100 also rotates in the positive (+) direction with respect to the pitch rotation axis (Y-axis) and rotates in the negative (−) direction with respect to the yaw rotation axis (Z-axis). Here, the rotation angle of the manipulation module 600 and the rotation angle of the end tool 100 may be variously set according to the proportions of the pulleys. In addition, when the actuation manipulation part is closed in a state in which the manipulation module 600 rotates in the positive (+) or negative (−) direction with respect to the pitch rotation shaft (Y-axis) and rotates in the positive (+) or negative (−) direction with respect to the yaw rotation shaft (Z-axis), the jaws 101 and 102 are also closed to perform an actuation motion.

### <Manipulation Module of Surgical Instrument>

Hereinafter, a process in which a force with which the user manipulates the manipulation module 600 is transmitted to the instrument module 30 will be described in detail. In detail, a process in which manipulation of the manipulation module 600 by the user implements an intuitive operation of the instrument module 30 will be described in detail.

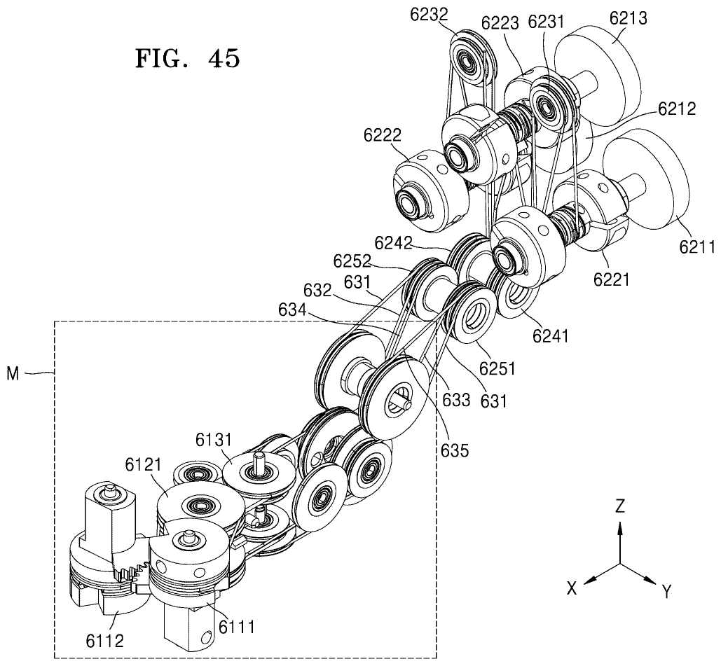

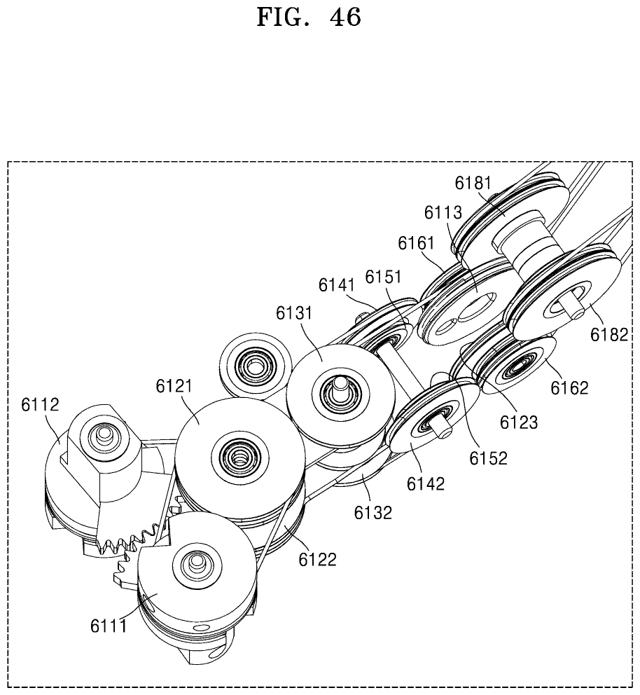

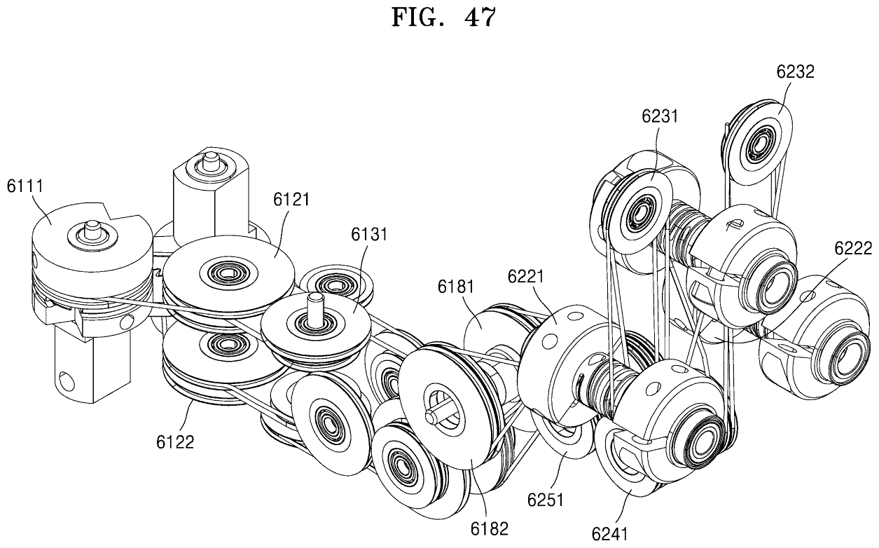

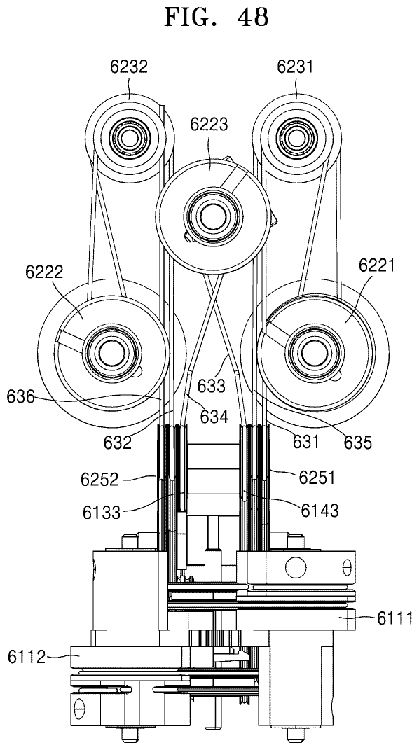

FIGS. 45 to 48 are diagrams for describing an arrangement structure of pulleys and wires inside the manipulation module 600 of FIG. 27.

Referring to FIGS. 45 to 48, the manipulation module 600 connects a part where the user inputs various manipulations (e.g., the handle 602, or the actuation manipulation part 603) to the instrument module 30, so as to serve to transmit a driving force of the manipulation module 600 to the instrument module 30, and may include a plurality of wires, pulleys, links, joints, gears, and the like.

The manipulation module 600 may include a pulley 6111, a pulley 6121, a pulley 6131, a pulley 6141, a pulley 6151, a pulley 6161, a pulley 6171, a pulley 6181, a pulley 6221, and a pulley 6231, which are associated with a rotational motion of the first jaw 101. In addition, the manipulation module 600 may include a pulley 6112, a pulley 6122, a pulley 6132, a pulley 6142, a pulley 6152, a pulley 6162, a pulley 6172, a pulley 6182, a pulley 6222, and a pulley 6232, which are associated with a rotational motion of the second jaw 102. In addition, the manipulation module 600 may include a pulley 6113, a pulley 6123, a pulley 6133, and a pulley 6143, which are associated with a pitch motion.

Here, although the drawings illustrate that the pulleys facing each other are arranged in parallel to each other, the present disclosure is not limited thereto, and the pulleys may be formed in various positions and sizes suitable for the configuration of the manipulation module 600.

In addition, the pulleys may be configured as one pulley, or a plurality of pulleys (e.g., two pulleys) may be arranged in parallel. For example, in a case in which two pulleys are arranged in parallel, different wires may be wound around each pulley. As a specific example, in a case in which two pulleys are arranged in parallel, a pair of wires constituting the first jaw 101 wire of the manipulation module 600 may be wound around each pulley.

Here, a rotation shaft may be arranged in each pulley. Pulleys may be fit into the respective rotation shafts, and as the pulleys rotate around the rotation shafts, the wires wound around the pulleys may be wound or unwound.

The pulley 6111 may function as a manipulation module 600 first jaw 101 actuation pulley, the pulley 6121 functions as a manipulation module 600 second jaw 102 actuation pulley, and these components may be collectively referred to as a manipulation module actuation pulley.

The pulley 6121 may function as a manipulation module first jaw 101 yaw main pulley, the pulley 6122 may function as a manipulation module second jaw 102 yaw main pulley, and these components may be collectively referred to as a manipulation module yaw main pulley.

The pulley 6131 may function as a manipulation module first jaw 101 yaw sub-pulley, the pulley 6132 may function as a manipulation module second jaw 102 yaw sub-pulley, and these components may be collectively referred to as a manipulation module yaw sub-pulley.

The pulley 6141 and the pulley 6151 may function as manipulation module first jaw pitch sub-pulleys, the pulley 6142 and the pulley 6152 may function as manipulation module second jaw pitch sub-pulleys, and these components may be collectively referred to as a manipulation module pitch sub-pulley.

The pulley 6161 may function as a manipulation module first jaw pitch main pulley, the pulley 6162 may function as a manipulation module second jaw pitch main pulley, and these components may be collectively referred to as a manipulation module pitch main pulley.

The pulley 6113 and the pulley 6123 function as a pitch power transmission pulley 6223, and the pulley 6133 and the pulley 6143 function as a manipulation module pitch sub-pulley.

A pulley 6241, a pulley 6242, the pulley 6231, and the pulley 6232 function as relay pulleys that change the paths of wires wound therearound.

The pulley 6221 functions as a first jaw power transmission pulley 6221, the pulley 6222 functions as a second jaw power transmission pulley 6222, and the pulley 6223 functions as a pitch power transmission pulley 6223.

Here, the first jaw power transmission pulley 6221 may be connected to the first jaw power transmission member 6211 to rotate together. In other words, it may also be described that, when the first jaw power transmission pulley 6221 rotates, the first jaw power transmission member 6211 connected to the first jaw power transmission pulley 6221 rotates together.

In addition, the second jaw power transmission pulley 6222 may be connected to the second jaw power transmission member 6212 to rotate together. In other words, it may also be described that, when the second jaw power transmission pulley 6222 rotates, the second jaw power transmission member 6212 connected to the second jaw power transmission pulley 6222 rotates together.

In addition, the pitch power transmission pulley 6223 may be connected to the pitch power transmission member 6213 to rotate together. In other words, it may also be described that, when the pitch power transmission pulley 6223 rotates, the pitch power transmission member 6213 connected to the pitch power transmission pulley 6223 rotates together.

The power connection part 620 includes a plurality of wires 631, 632, 633, 634, 635, and 636.

In detail, the power connection part 620 may include the wires 631 and 635, which are associated with a rotational motion of the first jaw 101. The wires 631 and 635 may be wound around the pulley 6111, the pulley 6121, the pulley 6131, the pulley 6141, the pulley 6151, the pulley 6161, the pulley 6171, the pulley 6181, the pulley 6221, and the pulley 6231, which are associated with a rotational motion of the first jaw 101. Here, the wire 631 and the wire 635 may be referred to as a manipulation module 600 first jaw 101 wire.

The power connection part 620 may include the wires 632 and 636, which are associated with a rotational motion of the second jaw 102. The wires 632 and 636 may be wound around the pulley 6112, the pulley 6122, the pulley 6132, the pulley 6142, the pulley 6152, the pulley 6162, the pulley 6172, the pulley 6182, the pulley 6222, and the pulley 6232, which are associated with a rotational motion of the second jaw 102. Here, the wire 632 and the wire 636 may be referred to as a manipulation module 600 second jaw 102 wire.

The power connection part 620 may include the wires 633 and 634, which are associated with a pitch motion of the end tool 100. The wires 633 and 634 may be wound around the pulley 6113, the pulley 6123, the pulley 6133, and the pulley 6143, which are associated with a pitch motion of the end tool 100. Here, the wire 633 and the wire 634 may be referred to as a manipulation module 600 pitch wire.

Hereinafter, an actuation motion, a yaw motion, and a pitch motion of the instrument module 30 will be described based on the connection and operation of the above-described pulleys and wires.

First, the actuation motion will be described below.

When the user inserts his or her fingers into the first actuation manipulation part 6031 and the second actuation manipulation part 6032, and rotates, with any one or both of the fingers, the first actuation manipulation part 6031 and the second actuation manipulation part 6032 in a direction in which they come closer to each other, the pulley 6111 and the pulley 6112 rotate in opposite directions, and the wire 631 and the wire 635 wound around the pulley 6111, and the wire 632 and the wire 636 wound around the pulley 6112 move to correspond to the rotation directions of the pulley 6111 and the pulley 6112.

As the wire 631 and the wire 635 move, the pulley 6121, the pulley 6131, the pulley 6141, the pulley 6151, the pulley 6161, the pulley 6171, the pulley 6181, and the pulley 6231, around which the wire 631 and the wire 635 are wound, rotate together. Accordingly, the pulley 6221, which is the first jaw power transmission pulley 6221, may rotate to rotate the first jaw power transmission member 6211, and thus, the first jaw 101 of the end tool 100 may rotate. Similarly, as the wire 632 and the wire 636 move, the pulley 6122, the pulley 6132, the pulley 6142, the pulley 6152, the pulley 6162, the pulley 6172, the pulley 6182, and the pulley 6232, around which the wire 632 and the wire 636 are wound, rotate together. Accordingly, the pulley 6222, which is the second jaw power transmission pulley 6222, may rotate to rotate the second jaw power transmission member 6212, and thus, the second jaw 102 of the end tool 100 may rotate. In this case, because the rotation directions of the pulleys 6111 and 6112 are opposite to each other, the first jaw 101 and the second jaw 102 also rotate in opposite directions. Thus, as the first jaw 101 and the second jaw 102 rotate simultaneously as described above, the two jaws 103 perform an actuation motion.

On the contrary, when the first actuation manipulation part 6031 and the second actuation manipulation part 6032 are rotated in a direction in which they move away from each other, the first jaw 101 and the second jaw 102 rotate a direction in which they move away from each other, such that the end tool 100 is opened.

Next, the yaw motion will be described below.

When the user rotates the handle 602 in the direction r2 of FIG. 28 while holding the handle 602, at least one area of the manipulation module 600 rotates in the same direction. In detail, when the user rotates the manipulation module 600 in the direction r2 while holding the handle 602, the pulley 6111 and the pulley 6112 rotate in the same direction, and the wire 631 and the wire 635 wound around the pulley 6111, and the wire 632 and the wire 636 wound around the pulley 6112 move to correspond to the rotation directions of the pulley 6111 and the pulley 6112.

As the wire 631 and the wire 635 move, the pulley 6121, the pulley 6131, the pulley 6141, the pulley 6151, the pulley 6161, the pulley 6171, the pulley 6181, and the pulley 6231, around which the wire 631 and the wire 635 are wound, rotate together. Accordingly, the pulley 6221, which is the first jaw power transmission pulley 6221, may rotate to rotate the first jaw power transmission member 6211, and thus, the first jaw 101 of the end tool 100 may rotate. Similarly, as the wire 632 and the wire 636 move, the pulley 6122, the pulley 6132, the pulley 6142, the pulley 6152, the pulley 6162, the pulley 6172, the pulley 6182, and the pulley 6232, around which the wire 632 and the wire 636 are wound, rotate together. Accordingly, the pulley 6222, which is the second jaw power transmission pulley 6222, may rotate to rotate the second jaw power transmission member 6212, and thus, the second jaw 102 of the end tool 100 may rotate. In this case, because the rotation directions of the pulleys 6111 and 6112 are the same, the first jaw 101 and the second jaw 102 also rotate in the same direction. Thus, as the first jaw 101 and the second jaw 102 rotate simultaneously as described above, the two jaws 103 may perform a yaw motion.

Next, the pitch motion will be described below.

When the user rotates the handle 602 in the direction r1 of FIG. 27 while holding the handle 602, at least one area of the manipulation module 600 rotates in the same direction. In detail, when the user rotates the manipulation module 600 in the direction r1 while holding the handle 602, the manipulation part 610 rotates in the direction r1 with respect to the power connection part 620. At this time, when the manipulation part 610 rotates in the direction r1, the pulley 6113 and the pulley 6123 rotate in the same direction, and the wire 634 wound around the pulley 6113 and the wire 633 wound around the pulley 6123 move to correspond to the rotation directions of the pulley 6113 and the pulley 6123, respectively.

As the wire 634 and the wire 633 move, the pulley 6133 around which the wire 634 is wound rotates in the opposite direction to the pulley 6113, and the pulley 6143 around which the wire 633 is wound rotates in the opposite direction to the pulley 6123. That is, the pulley 6133 and the pulley 6143 rotate in the same direction. At this time, because the wire 633 and the wire 634 are wound around the pitch power transmission pulley 6223, the pitch power transmission pulley 6223 rotates as the wire 633 and the wire 634 rotate. Accordingly, the pitch power transmission pulley 6223 may rotate to rotate the pitch power transmission member 6213, and thus, the end tool 100 pitch-rotates.

As described above, the power connection part 620 may further include at least one relay pulley.

The relay pulley may change the paths of wires wound therearound.

First, with respect to FIG. 45, the pulley 6133 and the pulley 6143 may be arranged to be lower than the manipulation module 600 jaw pulley and the pitch power transmission pulley 6223 (in the negative (−) direction of the Z-axis). The pulley 6133 and the pulley 6143 may change all of the paths of a plurality of wires 631, 632, 633, 634, 635, and 636, to be upward (the positive (+) direction of the Z-axis). Here, the wire 633 and the wire 634, which are pitch wires, may approach the pitch power transmission pulley 6223 from the pulley 6133 and the pulley 6143. The wire 631, the wire 632, the wire 635, and the wire 636, which are manipulation module 600 jaw wires, may extend to the pulley 6231 and the pulley 6232, which are other relay pulleys arranged to be higher than the pulley 6221 and the pulley 6222 that are manipulation module 600 jaw pulleys, and then be wound around the pulley 6231 and the pulley 6232.

In detail, the wire 631 and the wire 635 constituting the manipulation module 600 first jaw 101 wire may be wound around the pulley 6231. The pulley 6231 may change again the paths of the wire 631 and the wire 635 to be downward again such that the wire 631 and the wire 635 enter the first jaw power transmission pulley 6221. Similarly, the wire 632 and the wire 636 constituting the manipulation module 600 second jaw 102 wire may be wound around the pulley 6232. The pulley 6232 may change the paths of the wire 632 and the wire 636 to be downward again such that the wire 632 and the wire 637 enter the second jaw power transmission pulley 6222.

As such, by arranging the relay pulleys, the lengths of the wires 631, 632, 633, 634, 635, and 636 may be secured, and the wires 631, 632, 633, 634, 635, and 636 may approach the respective pulleys 6221, 6222, and 6332 almost perpendicularly.

### Examples of use of surgical instrument

Hereinafter, examples of use of the instrument module 30 according to the present disclosure will be described.

The instrument module 30 according to the present disclosure may be configured in a modular form and then selectively mounted on the robotic arm unit 21, 22, or 23 of a surgical robot, and the hand-held manipulation module 600. This means that, the instrument module 30 may be operated by a surgical robot or may be operated manually by a user manipulating the manipulation module 600, as necessary. In other words, this means that the instrument module 30, which is designed to be mounted and used on a surgical robot, may also be mounted and used on the manipulation module 600 that is modularized for reuse.

The instrument module 30 according to the present disclosure may be formed to be attachable to and detachable from the robotic arm unit 21, 22, or 23 and the manipulation module 600. For example, the user may separate the instrument module 30 from the robotic arm unit 21, 22, or 23 during surgery, and then connect the separated instrument module 30 to the manipulation module 600 to continue the surgery.

The instrument module 30 according to the present disclosure may be any one selected from among a surgical clamp, a grasper, a vessel sealer, and a stapler. In detail, the instrument module 30 according to the present disclosure may be variously implemented to perform a function of at least one of a surgical clamp, a grasper, a vessel sealer, a stapler, and a cauterizer (e.g., a monopolar hook or a spatula), and various instrument modules 30 may be selectively mounted and used on the robotic arm unit 21, 22, or 23 and the manipulation module 600. For example, when the user needs a clamp, an instrument module 30, which performs the function of a clamp, may be mounted on the robotic arm unit 21, 22, or 23 or the manipulation module 600 to perform surgery. Alternatively, when the user needs a grasper, an instrument module 30, which performs the function of a grasper, may be mounted on the robotic arm unit 21, 22, or 23 or the manipulation module 600 to perform surgery.

As such, the surgical robotic system 1 and the instrument module 30 according to the present disclosure may be separated from the surgical robot or the manipulation module 600, and then stored, which has the effect of saving space when stored.

In addition, the instrument module 30 according to the present disclosure is compatible with a surgical robot and the manipulation module 600, and thus, there is no need to provide various types of hand-held instruments. That is, according to the present disclosure, a user may replace the instrument module 30 mounted on a surgical robot or the manipulation module 600 with various types of modular instrument modules 30, and use them. Furthermore, because the manipulation module 600 is reusable, the manufacturing costs of the manipulation module 600 and the instrument module 30 are reduced.

In addition, according to the present disclosure, the instrument module 30 is compatible with a surgical robot and the manipulation module 600, and thus, even when an abnormality occurs in a surgical robot during surgery, it is possible to continue the surgery by mounting the instrument module 30 on the manipulation module 600 without stopping the surgery. Thus, there is the effect of improving the sustainability of surgery and the possibility of success of surgery, and simultaneously reducing the surgery time. Furthermore, a surgical method may be selected from among a robotic surgical method or a hand-held method according to a patient's condition, and thus, there is the effect of providing flexibility in a surgical procedure.

Although the present disclosure has been described with reference to the embodiment shown in the drawings, which is merely exemplary, it will be understood by those skilled in the art that various modifications and equivalent other embodiments are possible therefrom. Accordingly, the true technical protection scope of the present disclosure should be defined by the technical spirit of the appended claims.

The particular implementations shown and described herein are illustrative examples of the embodiments and are not intended to otherwise limit the scope of the embodiments in any way. For the sake of brevity, related-art electronics, control systems, software and other functional aspects of the systems may not be described in detail. Furthermore, line connections or connection members between elements depicted in the drawings represent functional connections and/or physical or circuit connections by way of example, and in actual applications, they may be replaced or embodied with various suitable additional functional connections, physical connections, or circuit connections. In addition, no item or component is essential to the practice of the disclosure unless the item or component is specifically described as being “essential” or “critical”.

The use of the terms “a” and “an” and “the” and similar referents in the context of describing the present disclosure (especially in the context of the following claims) are to be construed to cover both the singular and the plural. Further, recitation of ranges of values herein are merely intended to serve as a shorthand method of referring individually to each separate value falling within the range, unless otherwise indicated herein, and each separate value is incorporated into the specification as if it were individually recited herein. Finally, operations of all methods described herein can be performed in any suitable order unless otherwise indicated herein or otherwise clearly contradicted by context. The embodiments are not limited to the described order of the operations. The use of any and all examples, or exemplary terms (e.g., “such as”) provided herein, is intended merely to better illuminate the present disclosure and does not pose a limitation on the scope of the present disclosure unless otherwise claimed. Also, numerous modifications and adaptations will be readily apparent to those skilled in the art without departing from the spirit and scope of the present disclosure.

The present disclosure may provide a manipulation module for a surgical instrument in which a manipulation part for a surgical instrument is modularized to be reusable, enabling use of the same surgical instrument as a surgical robot.

In addition, the present disclosure may provide a surgical robotic system in which a surgical instrument and a manipulation part are configured in a detachable modular form, and a surgical instrument.

Accordingly, the present disclosure may provide case of storage, reusability of a manipulation part, and expandability to use various surgical instruments.

However, the above effects are only examples, and the effects of the present disclosure are not limited thereto.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A manipulation module for a surgical instrument, the manipulation module being configured to be coupled to an instrument module comprising an end tool having a pair of jaws formed to be rotatable, the manipulation module comprising:
a manipulation part configured to enable a user to perform a manipulation to control a motion of the end tool; and
a power connection part, which is connected to one side of the manipulation part, and detachably coupled to the instrument module to transmit a driving force for controlling the motion of the end tool to the instrument module, wherein the driving force is generated based on the manipulation performed by the user.

2. The manipulation module of claim 1, wherein the manipulation part is hinge-coupled to the power connection part to be rotatable in an upward or downward direction with respect to the power connection part by a manipulation by the user, and
when the manipulation part rotates in the upward or downward direction with respect to the power connection part, the end tool pitch-rotates.

3. The manipulation module of claim 1, wherein the power connection part comprises a coupling area coupled to the instrument module, and
the driving force is transmitted in the coupling area.

4. The manipulation module of claim 3, wherein the power connection further comprises at least one power transmission member arranged to overlap the coupling area at least in part, and configured to transmit the driving force to the instrument module.

5. The manipulation module of claim 4, wherein the instrument module further comprises at least one driving member configured to be coupled to a surgical robot to receive power from the surgical robot, and control the motion of the end tool by the received power, and
the power transmission member is coupled to the driving member in a one-to-one correspondence.

6. The manipulation module of claim 4, wherein the power transmission member is configured to, when the manipulation part is manipulated by the user, rotate in at least one direction.

7. The manipulation module of claim 6, wherein the power transmission member comprises a plurality of power transmission members configured to rotate on a same plane.

8. The manipulation module of claim 4, wherein the power connection part further comprises a jaw power transmission member coupled to the instrument module to transmit, to the instrument module, a driving force for controlling a rotational motion of the pair of jaws.

9. The manipulation module of claim 4, wherein the power connection part further comprises a pitch power transmission member coupled to the instrument module to transmit, to the instrument module, a driving force for controlling a pitch motion of the end tool.

10. The manipulation module of claim 1, further comprising a guide hole formed such that a shaft-shaped connection part provided in the instrument module is inserted therein, and configured to guide a coupling direction of the instrument module.

11. A surgical instrument comprising:
an instrument module comprising an end tool having a pair of jaws formed to be rotatable, and a driving part configured to control a motion of the end tool; and
a manipulation module detachably coupled to the instrument module and configured to transmit, to the driving part, a driving force for controlling the motion of the end tool.

12. The surgical instrument of claim 11, wherein the manipulation module comprises:
a manipulation part configured to enable a user to perform a manipulation to control the motion of the end tool; and
a power connection part that has one side connected to the manipulation part and another side detachably connected to the driving part, and is configured to transmit, to the driving part, a driving force generated based on a manipulation by the user.

13. The surgical instrument of claim 12, wherein the power connection part comprises a coupling area coupled to the driving part, and
the driving force is transmitted in the coupling area.

14. The surgical instrument of claim 13, wherein the power connection part further comprises at least one power transmission member arranged to overlap the coupling area at least in part, and configured to transmit the driving force to the driving part, and
the driving part comprises at least one driving member coupled to the power transmission member to receive the driving force from the power transmission member.

15. The surgical instrument of claim 14, wherein the power transmission member is configured to, when the manipulation part is manipulated by the user, rotate in at least one direction, and
the driving member is configured to be engaged with the power transmission member to rotate together with the power transmission member.

16. The surgical instrument of claim 15, wherein the power transmission member comprises a plurality of power transmission members configured to rotate on a same plane, and
the driving member comprises a plurality of driving members configured to rotate on a same plane.

17. The surgical instrument of claim 14, wherein the power transmission member and the driving member are coupled to each other in a one-to-one correspondence.

18. The surgical instrument of claim 14, wherein the power connection part further comprises a jaw power transmission member configured to transmit, to the driving part, a driving force for controlling a rotational motion of the pair of jaws, and
the driving part further comprises a jaw driving member coupled to the jaw power transmission member to receive, from the jaw power transmission member, the driving force for controlling the rotational motion of the pair of jaws.

19. The surgical instrument of claim 14, wherein the power connection part further comprises a pitch power transmission member configured to transmit, to the driving part, a driving force for controlling a pitch motion of the end tool, and
the driving part further comprises a pitch driving member coupled to the pitch power transmission member to receive, from the pitch power transmission member, the driving force for controlling the pitch motion of the end tool.

20. The surgical instrument of claim 11, wherein the instrument module further comprises a connection part formed to extend from the driving part toward the end tool, and
the manipulation module further comprises a guide hole formed such that the connection part is inserted therein, and configured to guide a coupling direction of the instrument module.
